SEQUENCE LISTING (81 sequences)

SEQ ID NO: 1 - protein, 10 aa, Artificial Sequence
  Synthetic
GYTFTDHAIH

SEQ ID NO: 2 - protein, 17 aa, Artificial Sequence
  Synthetic
YVSPGNGDIKYNEKFKG

SEQ ID NO: 3 - protein, 7 aa, Artificial Sequence
  Synthetic
SYGSFDY

SEQ ID NO: 4 - protein, 10 aa, Artificial Sequence
  Synthetic
SASSSVSYVY

SEQ ID NO: 5 - protein, 7 aa, Artificial Sequence
  Synthetic
RTSNLAS

SEQ ID NO: 6 - protein, 9 aa, Artificial Sequence
  Synthetic
QQYHSYPFT

SEQ ID NO: 7 - protein, 116 aa, Artificial Sequence
  Synthetic
DTMLQQSDAELVKPGASVKMSCKASGYTFTDHAIHWVKQKPEQGLEWIGYVSPGNGDIKYNEKFKGKATLSADKSSSTAYMQLNSLTSEDSAVYFCKRSYGSFDYWGQGTTLTVSS

SEQ ID NO: 8 - protein, 107 aa, Artificial Sequence
  Synthetic
QIVLTQSPAIMSSSPGEKVALSCSASSSVSYVYWYQQKPGSSPKPWIYRTSNLASGVPARFSGSGSGTSYSLTISSMEAEDAATYYCQQYHSYPFTFGSGTKLEIKR

SEQ ID NO: 9 - protein, 571 aa, Artificial Sequence
  Synthetic
DTMLQQSDAELVKPGASVKMSCKASGYTFTDHAIHWVKQKPEQGLEWIGYVSPGNGDIKYNEKFKGKATLSADKSSSTAYMQLNSLTSEDSAVYFCKRSYGSFDYWGQGTTLTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 10 - protein, 213 aa, Artificial Sequence
  Synthetic
QIVLTQSPAIMSSSPGEKVALSCSASSSVSYVYWYQQKPGSSPKPWIYRTSNLASGVPARFSGSGSGTSYSLTISSMEAEDAATYYCQQYHSYPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 446 aa, Artificial Sequence
  Synthetic
DTMLQQSDAELVKPGASVKMSCKASGYTFTDHAIHWVKQKPEQGLEWIGYVSPGNGDIKYNEKFKGKATLSADKSSSTAYMQLNSLTSEDSAVYFCKRSYGSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 213 aa, Artificial Sequence
  Synthetic
QIVLTQSPAIMSSSPGEKVALSCSASSSVSYVYWYQQKPGSSPKPWIYRTSNLASGVPARFSGSGSGTSYSLTISSMEAEDAATYYCQQYHSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1713 bp, Artificial Sequence
  Synthetic
gatacaatgctccagcagtccgacgccgaactggttaagcctggcgcctctgtgaagatgagctgcaaggccagcggctacaccttcacagatcacgccatccactgggtcaagcagaagcctgaacagggcctcgaatggatcggctacgttagccctggcaacggcgacatcaagtacaacgagaagttcaagggcaaagccacactgagcgccgacaagagcagcagcacagcctacatgcagctgaacagcctgacaagcgaggacagcgccgtgtacttctgcaagagaagctacggcagcttcgactactggggccagggcacaacactgacagtgtctagcgagagtcagtccttcccaaatgtcttccccctcgtctcctgcgagagccccctgtctgataagaatctggtggccatgggctgcctggcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacagagatctgcatgtgcccattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatgaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactggtaaacccacactgtacaatgtctccctgatcatgtctgacacaggcggcacctgctat

SEQ ID NO: 14 - DNA, 639 bp, Artificial Sequence
  Synthetic
cagatcgtgctgacacagagccccgccattatgtctagcagccctggcgaaaaggtggccctgagctgttctgccagcagcagcgtgtcctacgtgtactggtatcagcagaagcccggcagcagccccaagccttggatctacagaacaagcaatctggccagcggcgtgccagccagattttctggttctggcagcggcaccagctacagcctgacaatctctagcatggaagccgaggacgccgccacctactactgtcagcagtaccacagctaccccttcacctttggctccggcaccaagctggaaatcaagagagcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 15 - DNA, 1338 bp, Artificial Sequence
  Synthetic
gatacaatgctccagcagtccgacgccgaactggttaagcctggcgcctctgtgaagatgagctgcaaggccagcggctacaccttcacagatcacgccatccactgggtcaagcagaagcctgaacagggcctcgaatggatcggctacgttagccctggcaacggcgacatcaagtacaacgagaagttcaagggcaaagccacactgagcgccgacaagagcagcagcacagcctacatgcagctgaacagcctgacaagcgaggacagcgccgtgtacttctgcaagagaagctacggcagcttcgactactggggccagggcacaacactgacagtgtctagcgcctctacaaagggccctagcgttttcccactggctcctagcagcaagagcacatctggtggaacagccgctctgggctgcctggtcaaggattactttcctgagcctgtgaccgtgtcctggaattctggtgctctgaccagcggcgtgcacacatttccagccgtgctgcagtctagcggcctgtactctctgtctagcgtggtcacagtgcctagctctagcctgggcacccagacctacatctgcaacgtgaaccacaagcctagcaacaccaaggtggacaagaaggtggaacccaagagctgcgacaagacccacacctgtcctccatgtcctgctccagaactgctcggcggaccctccgttttcctgtttccacctaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacaccctgcctccaagccgggaagagatgaccaagaatcaggtgtccctgacctgcctcgtgaagggcttctacccttccgatatcgccgtggaatgggagagcaatggccagcctgagaacaactacaagacaacccctcctgtgctggactccgatggctcattcttcctgtacagcaagctgaccgtggacaagtccagatggcagcagggcaatgtgttctcctgctctgtgatgcacgaagccctgcacaaccactacacccagaagtctcttagcctgtctcctggcaaa

SEQ ID NO: 16 - DNA, 639 bp, Artificial Sequence
  Synthetic
cagatcgtgctgacacagagccccgccattatgtctagcagccctggcgaaaaggtggccctgagctgttctgccagcagcagcgtgtcctacgtgtactggtatcagcagaagcccggcagcagccccaagccttggatctacagaacaagcaatctggccagcggcgtgccagccagattttctggttctggcagcggcaccagctacagcctgacaatctctagcatggaagccgaggacgccgccacctactactgtcagcagtaccacagctaccccttcacctttggctccggcaccaagctggaaatcaagagaacagtggccgctccgagcgtgttcatctttccaccaagcgacgagcagctgaagtctggcacagcctctgtcgtgtgcctgctgaacaacttctaccccagagaagccaaggtgcagtggaaggtggacaatgccctgcagagcggcaatagccaagagagcgtgaccgagcaggacagcaaggactctacctactctctgagcagcaccctgacactgagcaaggccgactacgagaagcacaaagtgtacgcctgcgaagtgacacaccagggcctgtctagccctgtgaccaagagcttcaaccggggcgagtgt

SEQ ID NO: 17 - DNA, 20 bp, Artificial Sequence
  Synthetic
tcaccatggtctcatctatc

SEQ ID NO: 18 - protein, 11 aa, Artificial Sequence
  Synthetic
  MOD_RES (6)..(6): GalNAcylation
ALMDETMKELK

SEQ ID NO: 19 - protein, 20 aa, Artificial Sequence
  Synthetic
  MOD_RES (9)..(9): GalNAcylation
  MOD_RES (12)..(12): GalNAcylation
TEALSLGRTSTPGPAQSTIS

SEQ ID NO: 20 - protein, 18 aa, Artificial Sequence
  Synthetic
  MOD_RES (6)..(6): GalNAcylation
  MOD_RES (8)..(8): GalNAcylation
LALENYITALQAVPPRPR

SEQ ID NO: 21 - protein, 21 aa, Artificial Sequence
  Synthetic
  MOD_RES (12)..(12): GalNAcylation
  MOD_RES (13)..(13): GalNAcylation
TTAAPTTRRPPTTAPSRVPGR

SEQ ID NO: 22 - protein, 21 aa, Artificial Sequence
  Synthetic
  MOD_RES (3)..(3): GalNAcylation
  MOD_RES (10)..(10): GalNAcylation
  MOD_RES (12)..(12): GalNAcylation
KPSGLNGEASKSQEMVHLVNK

SEQ ID NO: 23 - protein, 9 aa, Artificial Sequence
  Synthetic
  MOD_RES (7)..(7): GalNAcylation
LSSTWETGK

SEQ ID NO: 24 - protein, 7 aa, Artificial Sequence
  Synthetic
  MOD_RES (2)..(2): GalNAcylation
  MOD_RES (4)..(4): Oxidation
SSLMPLR

SEQ ID NO: 25 - protein, 17 aa, Artificial Sequence
  Synthetic
  MOD_RES (8)..(8): GalNAcylation
  MOD_RES (13)..(13): GalNAcylation
KALEGLQYPFAVTSYGK

SEQ ID NO: 26 - protein, 24 aa, Artificial Sequence
  Synthetic
  MOD_RES (19)..(19): GalNAcylation
  MOD_RES (22)..(22): GalNAcylation
MDLRQFLMCLSLCTAFALSKPTEK

SEQ ID NO: 27 - protein, 6 aa, Artificial Sequence
  Synthetic
  MOD_RES (3)..(3): GalNAcylation
YITMLK

SEQ ID NO: 28 - protein, 20 aa, Artificial Sequence
  Synthetic
  MOD_RES (2)..(2): GalNAcylation
  MOD_RES (14)..(14): GalNAcylation
HSMAYQDLHSEITSLFKDVF

SEQ ID NO: 29 - protein, 12 aa, Artificial Sequence
  Synthetic
  MOD_RES (6)..(6): GalNAcylation
  MOD_RES (11)..(11): GalNAcylation
ERQVATAVQWTK

SEQ ID NO: 30 - protein, 13 aa, Artificial Sequence
  Synthetic
  MOD_RES (10)..(10): GalNAcylation
  MOD_RES (12)..(12): GalNAcylation
LRARMEEMGSRTR

SEQ ID NO: 31 - protein, 20 aa, Artificial Sequence
  Synthetic
  MOD_RES (2)..(2): GalNAcylation
  MOD_RES (12)..(12): GalNAcylation
DTFNDSAAPQSTTWPETSPR

SEQ ID NO: 32 - protein, 27 aa, Artificial Sequence
  Synthetic
  MOD_RES (18)..(18): GalNAcylation
FLHQERMDVCETHLHWHTVAKETCSEK

SEQ ID NO: 33 - protein, 9 aa, Artificial Sequence
  Synthetic
  SITE (1)..(9): Not identified site-specific GalNAcylation
TTAAPTTRR

SEQ ID NO: 34 - protein, 10 aa, Artificial Sequence
  Synthetic
  SITE (1)..(10): Not identified site-specific GalNAcylation
SQEMVHLVNK

SEQ ID NO: 35 - protein, 9 aa, Artificial Sequence
  Synthetic
  MOD_RES (7)..(7): GalNAcylation
EHPCVESMK

SEQ ID NO: 36 - protein, 34 aa, Artificial Sequence
  Synthetic
  MOD_RES (3)..(3): GalNAcylation
  MOD_RES (16)..(16): GalNAcylation
GCSLEQISMELAKVNYVKNNICFMSHHQEELVPK

SEQ ID NO: 37 - protein, 11 aa, Artificial Sequence
  Synthetic
  MOD_RES (9)..(9): GalNAcylation
IEVAKLDGTQR

SEQ ID NO: 38 - protein, 19 aa, Artificial Sequence
  Synthetic
  MOD_RES (10)..(10): GalNAcylation
IDGDKDGFVTVDELKDWIK

SEQ ID NO: 39 - protein, 6 aa, Artificial Sequence
  Synthetic
  MOD_RES (3)..(3): GalNAcylation
IMSCCK

SEQ ID NO: 40 - protein, 13 aa, Artificial Sequence
  Synthetic
  MOD_RES (5)..(5): GalNAcylation
  MOD_RES (6)..(6): GalNAcylation
CAFGYSGLDCKDK

SEQ ID NO: 41 - protein, 19 aa, Artificial Sequence
  Synthetic
  MOD_RES (7)..(7): GalNAcylation
  MOD_RES (9)..(9): GalNAcylation
  MOD_RES (12)..(12): GalNAcylation
HPGNFSSLSCDYFAVNQSR

SEQ ID NO: 42 - protein, 20 aa, Artificial Sequence
  Synthetic
  MOD_RES (1)..(1): GalNAcylation
SWFEPLVEDMQRQWAGLVEK

SEQ ID NO: 43 - protein, 17 aa, Artificial Sequence
  Synthetic
  MOD_RES (10)..(10): GalNAcylation
  MOD_RES (12)..(12): GalNAcylation
ISTSAPLSSSASVLDNK

SEQ ID NO: 44 - protein, 10 aa, Artificial Sequence
  Synthetic
  SITE (1)..(10): Not identified site-specific GalNAcylation
PAADRGLTTR

SEQ ID NO: 45 - protein, 7 aa, Artificial Sequence
  Synthetic
  SITE (1)..(7): Not identified site-specific GalNAcylation
KDFRSVR

SEQ ID NO: 46 - protein, 28 aa, Artificial Sequence
  Synthetic
  MOD_RES (15)..(15): GalNAcylation
RMDMDSSHSITLQPTANPNTGLVEDLDR

SEQ ID NO: 47 - protein, 14 aa, Artificial Sequence
  Synthetic
  MOD_RES (4)..(4): GalNAcylation
  MOD_RES (7)..(7): GalNAcylation
IFPYEEYASWKTEK

SEQ ID NO: 48 - protein, 38 aa, Artificial Sequence
  Synthetic
  MOD_RES (1)..(1): GalNAcylation
  MOD_RES (23)..(23): GalNAcylation
YMFGLVIPLGVLQAIAMYFLPPSPRFLVMKGQEGAASK

SEQ ID NO: 49 - protein, 11 aa, Artificial Sequence
  Synthetic
  SITE (1)..(11): Not identified site-specific GalNAcylation
IESALLDGSER

SEQ ID NO: 50 - protein, 13 aa, Artificial Sequence
  Synthetic
  SITE (1)..(13): Not identified site-specific GalNAcylation
TFDQLTPEESKER

SEQ ID NO: 51 - protein, 7 aa, Artificial Sequence
  Synthetic
  MOD_RES (1)..(1): GalNAcylation
TCDILFR

SEQ ID NO: 52 - protein, 16 aa, Artificial Sequence
  Synthetic
  SITE (1)..(16): Not identified site-specific GalNAcylation
STGFTNLGAEGSVFPK

SEQ ID NO: 53 - protein, 11 aa, Artificial Sequence
  Synthetic
  SITE (1)..(11): Not identified site-specific GalNAcylation
RSLPYGTAMEK

SEQ ID NO: 54 - protein, 17 aa, Artificial Sequence
  Synthetic
YFSPGNGDIKYNEKFKG

SEQ ID NO: 55 - protein, 17 aa, Artificial Sequence
  Synthetic
HFSPGNGDIKYNEKFKG

SEQ ID NO: 56 - protein, 7 aa, Artificial Sequence
  Synthetic
SYGNYDY

SEQ ID NO: 57 - protein, 8 aa, Artificial Sequence
  Synthetic
SQPGKWDY

SEQ ID NO: 58 - protein, 8 aa, Artificial Sequence
  Synthetic
TSTFFFDY

SEQ ID NO: 59 - protein, 11 aa, Artificial Sequence
  Synthetic
RASQNIGTSIH

SEQ ID NO: 60 - protein, 11 aa, Artificial Sequence
  Synthetic
KASQDVSTTVA

SEQ ID NO: 61 - protein, 11 aa, Artificial Sequence
  Synthetic
LNSGDQKNYLT

SEQ ID NO: 62 - protein, 7 aa, Artificial Sequence
  Synthetic
YASESVS

SEQ ID NO: 63 - protein, 7 aa, Artificial Sequence
  Synthetic
SASYRYT

SEQ ID NO: 64 - protein, 7 aa, Artificial Sequence
  Synthetic
WASTRES

SEQ ID NO: 65 - protein, 9 aa, Artificial Sequence
  Synthetic
QHTNSWPTT

SEQ ID NO: 66 - protein, 9 aa, Artificial Sequence
  Synthetic
QQHYSTPPT

SEQ ID NO: 67 - protein, 9 aa, Artificial Sequence
  Synthetic
QNDYSYPLT

SEQ ID NO: 68 - protein, 8 aa, Artificial Sequence
  Synthetic
  SITE (4)..(5): May be modified by GalNAcylation
APGSTAPP

SEQ ID NO: 69 - protein, 16 aa, Artificial Sequence
  Synthetic
  SITE (9)..(9): May be modified by GalNAcylation
GTTPSPVPTTSTTSAP

SEQ ID NO: 70 - protein, 12 aa, Artificial Sequence
  Synthetic
  SITE (3)..(3): May be modified by GalNAcylation
  SITE (5)..(5): May be modified by GalNAcylation
  SITE (9)..(9): May be modified by GalNAcylation
KPSPSTPPTPSC

SEQ ID NO: 71 - protein, 12 aa, Artificial Sequence
  Synthetic
  SITE (3)..(3): May be modified by GalNAcylation
  SITE (4)..(4): May be modified by GalNAcylation
  SITE (7)..(7): May be modified by GalNAcylation
  SITE (9)..(9): May be modified by GalNAcylation
  SITE (11)..(11): May be modified by GalNAcylation
KPSTPPTPSPSC

SEQ ID NO: 72 - protein, 12 aa, Artificial Sequence
  Synthetic
  SITE (3)..(3): May be modified by GalNAcylation
  SITE (5)..(5): May be modified by GalNAcylation
  SITE (7)..(7): May be modified by GalNAcylation
  SITE (11)..(11): May be modified by GalNAcylation
KPTPSPSTPPTC

SEQ ID NO: 73 - protein, 12 aa, Artificial Sequence
  Synthetic
  SITE (5)..(5): May be modified by GalNAcylation
  SITE (6)..(6): May be modified by GalNAcylation
  SITE (9)..(9): May be modified by GalNAcylation
  SITE (11)..(11): May be modified by GalNAcylation
KPVPSTPPTPSC

SEQ ID NO: 74 - protein, 12 aa, Artificial Sequence
  Synthetic
  SITE (2)..(2): May be modified by GalNAcylation
  SITE (5)..(5): May be modified by GalNAcylation
  SITE (7)..(7): May be modified by GalNAcylation
  SITE (9)..(9): May be modified by GalNAcylation
  SITE (10)..(10): May be modified by GalNAcylation
KTPPTPSPSTPC

SEQ ID NO: 75 - protein, 4 aa, Artificial Sequence
  Synthetic
KTTT

SEQ ID NO: 76 - protein, 5 aa, Artificial Sequence
  Synthetic
KTTTG

SEQ ID NO: 77 - protein, 9 aa, Artificial Sequence
  Synthetic
  SITE (3)..(3): May be modified by GalNAcylation
  SITE (4)..(4): May be modified by GalNAcylation
  SITE (5)..(5): May be modified by GalNAcylation
  SITE (6)..(6): May be modified by GalNAcylation
PPTTTTKKP

SEQ ID NO: 78 - protein, 13 aa, Artificial Sequence
  Synthetic
  SITE (7)..(7): May be modified by GalNAcylation
PTTDSTTPAPTTK

SEQ ID NO: 79 - protein, 7 aa, Artificial Sequence
  Synthetic
  SITE (2)..(2): May be modified by GalNAcylation
  SITE (3)..(3): May be modified by GalNAcylation
  SITE (4)..(4): May be modified by GalNAcylation
PTTTPLK

SEQ ID NO: 80 - protein, 10 aa, Artificial Sequence
  Synthetic
  SITE (6)..(6): May be modified by GalNAcylation
TSAPDTRDAP

SEQ ID NO: 81 - protein, 14 aa, Artificial Sequence
  Synthetic
  SITE (11)..(11): May be modified by GalNAcylation
  SITE (12)..(12): May be modified by GalNAcylation
  SITE (13)..(13): May be modified by GalNAcylation
VAAWTLKAAATTTG

(19) United States
(12) Patent Application Publication
(10) Pub. No.: US 2024/0092935 A1
(43) Pub. Date: Mar. 21, 2024

(54) ANTI-TN ANTIBODIES AND USES THEREOF

(71) Applicant: Beth Israel Deaconess Medical Center, Inc., Boston, MA (US)

(72) Inventors: Richard D. Cummings, Brookline, MA (US); Elliot Chaikof, Newton, MA (US); Yasuyuki Matsumoto, Revere, MA (US); Matthew R. Kudelka, Decatur, GA (US)

(73) Assignee: Beth Israel Deaconess Medical Center, Inc., Boston, MA (US)

(21) Appl. No.: 17/767,766

(22) PCT Filed: Oct. 9, 2020
(86) PCT No.: PCT/US2020/055051

§ 371 Date: Apr. 8, 2022

Related U.S. Application Data
(60) Provisional application No. 62/914,343, filed on Oct. 11, 2019.

(51) Int. Cl. C07K 16/30; C07K 16/28; G01N 33/574

(52) U.S. Cl. CPC C07K 16/3076; C07K 16/2896; G01N 33/57492; C07K 2317/24; C07K 2317/40; C07K 2317/732; C07K 2317/734; C07K 2317/76

(57) ABSTRACT

The present disclosure provides anti-Tn antibodies (e.g., BaGs6 and/or Remab6) having superior specificity for Tn antigen on cancer cells. Also provided herein, are nucleic acids, vectors, or vector sets that encode the anti-Tn antibody.

### RELATED APPLICATIONS

This application claims priority under 35 U.S.C. § 119(e) to U.S. Provisional Application, U.S. Ser. No. 62/914,343, filed Oct. 11, 2019, entitled “ANTI-TN ANTIBODIES AND USES THEREOF,” which is incorporated by reference herein.

### FEDERALLY SPONSORED RESEARCH

This invention was made with government support under Grant No. CA168930 awarded by the National Institutes of Health. The government has certain rights in the invention.

### BACKGROUND

Tn antigen is a carbohydrate antigen defined as two or more N-acetylgalactosamine (GalNAc) monosaccharides in an O-glycosidic alpha-linkage individually linked to the amino acids threonine, serine or tyrosine in close proximity. The Tn antigen is a neoantigen abnormally expressed in many human carcinomas and expression correlates with metastasis and poor survival. To explore its biomarker potential, new antibodies are needed that specifically recognize this antigen on the cells of certain tumors. Currently available anti-Tn antibodies may cross-react with Tn on IgA1, STn, T glycopeptides, BGA, or glycans expressing terminal GalNAc. Such a lack of specificity poses a significant challenge in using such antibodies to specifically detect Tn antigen in vivo, and in using them to treat cancers expressing the Tn antigen.

### SUMMARY

The present disclosure is based, at least in part, on the development of anti-Tn antibodies (e.g., BaGs6 and/or Remab6), which possess unexpected superior features compared with known anti-Tn antibodies. Such superior features include at least the following: (i) these antibodies specifically recognize Tn antigen on tumor cells; and/or (ii) these antibodies do not cross-react with Tn antigen on circulating human IgA1.

In some aspects, the present disclosure provides an isolated antibody comprising: (i) a heavy chain variable domain (VH), which comprises a heavy chain complementary determining region 1 (HC CDR1) of SEQ ID NO: 1; a heavy chain complementary determining region 2 (HC CDR2) of SEQ ID NO: 2; and a heavy chain complementary determining region 3 (HC CDR3) of SEQ ID NO: 3. In some embodiments, the isolated antibody binds to Tn antigen.

Alternatively or in addition, the isolated antibody may comprise a light chain variable domain (VL), which comprises a light chain complementary determining region 1 (LC CDR1) of SEQ ID NO: 4; a light chain complementary determining region 2 (LC CDR2) of SEQ ID NO: 5; and a light chain complementary determining region 3 (LC CDR3) of SEQ ID NO: 6. In some embodiments, the isolated antibody binds to Tn antigen.

In some aspects, the present disclosure provides an isolated antibody that binds to Tn antigen binds the same epitope as BaGs6 or competes against BaGs6 for binding to the Tn antigen.

In some embodiments, the antibody comprises a HC CDR1, a HC CDR2, and a HC CDR3, which collectively contain no more than 10, 9, 8, 7, 5, 4, 3, 2, or 1 amino acid variations as compared with the HC CDR1, HC CDR2, and HC CDR3 of BaGs6.

Alliteratively or in addition, the antibody comprises a LC CDR1, LC CDR2, and LC CDR3, which collectively contains no more than 10, 9, 8, 7, 5, 4, 3, 2, or 1 amino acid variations as compared with the LC CDR1, LC CDR2, and LC CDR3 of BaGs6.

In some embodiments, the antibody comprises the same heavy chain complementary determining regions (HC CDRs) and/or the same light chain complementary determining regions (LC CDRs) as BaGs6. In some embodiments, the antibody comprises the same heavy chain complementary determining regions (HC CDRs) and the same light chain complementary determining regions (LC CDRs) as BaGs6. In some embodiments, the antibody comprises a heavy chain variable domain at least 85% identical to the heavy chain variable domain (VH) of BaGs6, and/or a light chain variable domain at least 85% identical to the light chain variable domain (VL) of BaGs6. In some embodiments, the antibody comprises a heavy chain variable domain at least 85% identical to amino acid sequence of SEQ ID NO: 7, and a light chain variable domain at least 85% identical to amino acid sequence of SEQ ID NO: 8.

In some embodiments, the antibody is a mouse IgM. In some embodiments, the antibody comprises a heavy chain at least 85% identical to amino acid sequence of SEQ ID NO: 9, and a light chain at least 85% identical to amino acid sequence of SEQ ID NO: 10.

In some embodiments, the antibody is a chimeric antibody. In some embodiments, the antibody is a chimeric antibody that comprises a human Fc portion. In some embodiments, the antibody comprises a heavy chain at least 85% identical to amino acid sequence of SEQ ID NO: 11, and a light chain at least 85% identical to amino acid sequence of SEQ ID NO: 12.

In some embodiments, the antibody specifically recognizes Tn antigen on tumor cells. In some embodiments, the antibody does not recognize Tn antigen on immunoglobulin A1 (IgA1). In some embodiments, the IgA1 is human IgA1.

In some embodiments, the antibody includes an afucosylated Fc fragment. In some embodiments, the antibody is present in an antibody population, and the antibody population comprises less than 50% fucosylated antibodies. In some embodiments, the antibody has increased Antibody-dependent Cellular Cytotoxicity (ADCC) activity compared to the same antibody that contains a fucosylated Fc fragment.

In some embodiments, the antibody is an antigen-binding fragment selected from Fab, Fab′, F(ab′)2, and Fv fragments. In some embodiments, the antibody is a single chain antibody, a bispecific antibody, or a nanobody.

In some embodiments, the antibody is conjugated to an active agent. In some embodiments, the antibody is conjugated to wherein the active agent is a particles, a Nanoparticles, a surface, a small Molecules, a peptide, an enzyme, an oligonucleotide, a detectable label, an imaging agent, or a therapeutic agent. In some embodiments, the imaging agent is a radioactive agent selected from the group consisting of fluorine-18, zirconium-89, copper-64, yttrium-86, indium-111, and iodine-124. In some embodiments, the therapeutic agent is a cytotoxic agent or a toxin. In some embodiments, the cytotoxic agent is selected from the group consisting of dolastin 10, zogamicin, monomethyl auristatin E (MMAE), cryptophycin and analogs thereof, enediyne antiobiotics including wesperamicin and maytansines (emtasine DM1/DM4), calicheamicin, capecitabine, lapatinib, anthracyclines, duocarmycins, and pyyrolobenzodiazepines. In some embodiments, the toxin is Pseudomonas exotoxin, or diphtheria toxin. In some embodiments, the detectable label is a fluorescent protein or a fluorescent compound.

In some aspects, the present disclosure provides a nucleic acid or a nucleic acid set, which collectively encode the isolated antibody described herein. In some embodiments, the nucleic acid is a vector or a vector set, wherein the vector is optionally an expression vector.

In some aspects, the present disclosure provides a host cell comprising the vector or vector set described herein. In some embodiments, the host cell is selected from the group consisting of bacterial cells, yeast cells, insect cells, plant cells, or mammalian cells. In some embodiments, the host cell are B cells. In some embodiments, the host cell are hybridoma.

In some aspects, the present disclosure provides a genetically engineered host cell comprising a double allele knock-out of a fucose synthase. In some embodiments, the fucose synthase is a GDP-L-fucose synthase. In some embodiments, the cells are capable of producing afucosylated antibodies in the absence of fucose in cell culture medium. In some embodiments, the cells are capable of producing fucosylated antibodies in the presence of fucose in cell culture medium. In some embodiments, the antibody is a therapeutic antibody. In some embodiments, the antibody is a non-therapeutic antibody. In some embodiments, the antibody is a diagnostic antibody. In some embodiments, the genetically engineered host cell described herein comprises a nucleic acid encoding the anti-Tn antibody as described herein. In some embodiments, the genetically engineered host cell produces an anti-Tn antigen antibody as described herein. In some embodiments, the genetically engineered host cell is a mammalian cell. In some embodiments, the mammalian cell is HEK293 cell, Chinese hamster ovary (CHO) cell, HeLa cell, HT-1080 cell, PER.C6, HKB-11 cell, CAP cell, HuH07 cell, NS0 cell, HKB11, Sp2/0 cell, BHK cell, or C127 cells.

In some aspects, the present disclosure provides a genetically engineered immune cell, which expresses a chimeric receptor comprising an extracellular domain and at least one cytoplasmic signaling domain, wherein the extracellular domain is a single chain antibody derived from an anti-Tn antibody as described herein. In some embodiments, the single chain antibody comprises a heavy chain variable domain and/or a light chain variable domain of an anti-Tn antibody as described herein. In some embodiments, the genetically engineered immune cell is a CAR-T cell.

In some aspects, the present disclosure provides a pharmaceutical composition comprising anti-Tn antibody as described herein, the nucleic acid encoding the anti-Tn antibody described herein, the host cell described herein, the genetically engineered host cell described herein, or the genetically engineered immune cell described herein, wherein the pharmaceutical composition optionally further comprises a pharmaceutically acceptable carrier.

In some aspects, the present disclosure provides a kit comprising an isolated anti-Tn antibody as described herein, a nucleic acid, vector or vector set encoding an anti-Tn antibody as described herein, a host cell as described herein, a genetically engineered host cell as described herein, a genetically engineered immune cell as described herein, or a pharmaceutical composition as described herein.

In some aspects, the present disclosure provides methods for producing an antibody that binds to human Tn antigen, the method comprising: (i) culturing a host cell described herein or a genetically engineered host cell of described herein in a medium for production of the antibody; and (ii) collecting the host cell or the medium for isolation of the antibody. In some embodiments, the method further comprises purifying the antibody from the host cell or the medium.

In some aspects, the present disclosure provides methods for detecting the presence of Tn antigen, the method comprising contacting an anti-Tn antigen antibody as described herein with a subject or a biological sample obtained from a subject suspected of containing Tn antigen, and determining binding of the anti-Tn antigen antibody to Tn antigen in the biological sample. In some embodiments, the antibody is conjugated to a detectable label. In some embodiments, the biological sample is in vivo, and the step of contacting is performed by administering to the subject an effective amount of the anti-Tn antigen antibody for detection.

In some aspects, the present disclosure provides methods for treating a cancer in a patient in need thereof, the method comprising administering to a subject an effective amount of an isolated anti-Tn antibody as described herein, a nucleic acid encoding an anti-Tn antibody described herein, the host cell described herein, a genetically engineered host cell as described herein, a genetically engineered immune cell as described herein, or a pharmaceutical composition as described herein. In some embodiments, the subject is a human patient having, suspected of having, or at risk for cancer. In some embodiments, the human patient has a cancer selected from the group consisting of colorectum cancer, breast cancer, prostate cancer, lung cancer, ovarian cancer, stomach cancer, bladder cancer, cervix cancer, pancreatic cancer, endometrial cancer, glioblastomas, salivary gland cancer, nasopharyngeal cancers, skin cancers, basal cell carcinomas, squamous cell carcinomas, renal cell carcinomas, ductal carcinomas, invasive ductal carcinomas, adenocarcinomas, esophageal cancer, unspecified gastrointestinal cancer, pancreatic cancer, and melanoma, as well as sarcomas, including angiosarcoma, bone sarcoma, osteosarcoma, neurofibrosarcomas, rhabdomyosarcoma, soft tissue sarcoma, synovial sarcoma, condrosarcoma, chordomas, Kaposi's sarcoma, giant cell tumor of the bone, leiomyosarcoma, desmoid-type fibromatosis, Ewing's sarcoma, fibroblastic sarcoma, gastrointestinal stromal tumors, lymphomas, leukemia, and thymomas.

In some aspects, the present disclosure provides a method for producing an afucosylated antibody comprising (i) transfecting a genetically engineered host cell described herein with a nucleic acid encoding an antibody (e.g., anti-Tn antibody as described herein); (ii) culturing the genetically engineered host cell in a medium for production of the antibody; and (iii) collecting the genetically engineered host cell or the medium for isolation of the antibody. In some embodiments, the antibody is a therapeutic antibody. In some embodiments, the antibody is the anti-Tn antibody as described herein.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF DRAWINGS

FIGS. 1A-1D show an overview of the experimental workflow and identification of complete amino acid sequence of BaGs6. FIG. 1A: mouse ascites BaGs6 (IgM) antibody, reactive to Tn antigen and used in a number of publications as a cancer biomarker, was purified using affinity chromatography with immobilized Asialo-BSM which carries a high density of Tn antigen. From complete amino acid sequences, the recombinant Tn antigen specific human IgG1, named Remab6, was purified and characterized. FIG. 1B: Asialo-BSM beads were prepared by neuraminidase treatment of BSM beads. High-density Asialo-BSM beads were stained with ascites or anti-STn antibody confirmed by microscopy analysis. FIG. 1C: BaGs6-containing mouse ascites were affinity-purified with Asialo-BSM beads. FIG. 1D: complete amino acid sequences were determined by proteomic de novo sequencing and CDRs of Heavy chain (top) and Light chain (bottom) were compared between three established anti-Tn antibodies (83D4, MLS128, and 5E5). * indicates identical residues in all four antibodies. BaGs6: HC CDR1 (SEQ ID NO: 1), HC CDR2 (SEQ ID NO: 2), HC CDR3 (SEQ ID NO: 3), LC CDR1 (SEQ ID NO: 4), LC CDR2 (SEQ ID NO: 5), LC CDR3 (SEQ ID NO: 6). 84D4: HC CDR1 (SEQ ID NO: 1), HC CDR2 (SEQ ID NO: 54), HC CDR3 (SEQ ID NO: 56), LC CDR1 (SEQ ID NO: 59), LC CDR2 (SEQ ID NO: 62), LC CDR3 (SEQ ID NO: 65). MLS128: HC CDR1 (SEQ ID NO: 1), HC CDR2 (SEQ ID NO: 54), HC CDR3 (SEQ ID NO: 57), LC CDR1 (SEQ ID NO: 60), LC CDR2 (SEQ ID NO: 63), LC CDR3 (SEQ ID NO: 66). 5E5: HC CDR1 (SEQ ID NO: 1), HC CDR2 (SEQ ID NO: 55), HC CDR3 (SEQ ID NO: 58), LC CDR1 (SEQ ID NO: 61), LC CDR2 (SEQ ID NO: 64), LC CDR3 (SEQ ID NO: 67).

FIGS. 2A-2G show Remab6 is specific for Tn glycopeptides, but not Tn on IgA1, STn, T glycopeptides, BGA, or glycans expressing terminal GalNAc. FIG. 2A: Remab6 and ReBaGs6 were recombinantly expressed in HEK293 freestyle expression system. The purified Remab6 and ReBaGs6 were separated by SDS-PAGE and stained by CBB solution. FIG. 2B: Tn glycopeptide (GP) array was probed with ReBaGs6 (middle) and Remab6 (right) to compare to the specificity of the original mouse ascites (left). Chart ID corresponds to Table 1.

FIG. 2C: Total cell extracts (TCE) and purified IgA from wild-type Dakiki cells (Tn−) versus Cosmc KO Dakiki cells (Tn+) were analyzed by Western blot using ReBaGs6 (left), Remab6 (middle), VVA (right-top), and goat anti-human IgA antibody (right-bottom). FIGS. 2D-2E: enzymatically remodeled Tn glycopeptide array slides to create STn (FIG. 2D) and T (FIG. 2E) antigen glycopeptides (ID1-8). STn and T glycopeptide arrays were probed with Remab6. Error bars represent ±1 SD of four replicates. FIG. 2F: Affinity constant was measured with ReBaGs6 (left), Remab6 (middle), and VVA (right) by Asialo-BSM-coated plate. Circle (non-treated), square (pretreated with 100 mM GalNAc), and triangle (pretreated with 100 mM GlcNAc) were plotted. Error bars represent SD of two replicates. RFU=relative fluorescence units. FIG. 2G shows total cell extracts (TCE) and purified IgA from wild-type Dakiki cells (Tn−) versus Cosmc KO Dakiki cells (Tn+) were analyzed by Western blot using ReBaGs6, Remab6, VVA, and goat anti-human IgA antibody.

FIGS. 3A-3C show binding profiles and distribution of Tn-carrying molecules within cells. FIG. 3A: flow cytometry profiles using Remab6 (top) and anti-STn antibody (bottom) with or without treatment with neuraminidase (Neu) on colorectal, breast, gastric carcinoma and leukemic cell lines. FIG. 3B: immunofluorescence studies showing localization of Tn positive staining with respect to nuclear (DAPI), cis-medial Golgi (Giantin; top), trans Golgi (TGN46; middle) and ER (Calnexin; bottom) of Tn-positive (left) and Tn-negative (right) in MDA-MB-231 cell line. Images were collected by confocal microscopy (Zeiss). FIG. 3C: replicate immunofluorescence images showing localization of Tn+ staining with respect to nuclear (DAPI), cis-medial Golgi (Giantin; top), trans Golgi (TGN46; middle) and ER (Calnexin; bottom) of Tn-positive (left) and Tn-negative (right) MDA-MB-231 cell line. Images were collected by confocal microscopy (Zeiss).

FIGS. 4A-4D show immunohistochemical staining in IEC-Cosmc KO mice and human cancer cell block sections, and human cancer tissue array. FIG. 4A: IHC staining with Remab6 using small intestine-colon-rectum sections in Villi-specific Cosmc KO mice (male; KO Cosmc^{-/y}, female; KO Cosmc^{+/-}) compared to WT. Scale bar represents 100 μm. FIG. 4B: cell block section staining with Remab6 of Tn-positive or Tn-negative populations of human carcinoma cell lines (LS174T, MDA-MB-231, and MKN-45). Scale bar represents 10 μm.

FIG. 4C: human cancer tissue array (FDA808k-1/2, US Biomax Inc) with Remab6-Fab-HRP reagent. Squares represent Tn positive staining sites with high magnification. Brown indicates Tn staining, and blue indicates nuclear staining. FIG. 4D: IHC staining in human cancer tissue array, FDA808k-1 and k-2. Human tumor tissue array with normal tissues (FDA808k-1 and k-2) stained with Remab6-Fab-HRP. Squares encloses Tn positive staining: in normal tissues, intracellular staining in stomach, small intestine, and colon tissues; in tumor tissues, 12 different tissues demonstrate staining with Remab6, as noted by squares and corresponding with Table 3.

FIGS. 5A-5B show LC-MS analysis to identify Tn containing glycoproteins in colorectal carcinoma cell line. FIG. 5A: immunoprecipitated glycoproteins with ReBaGs6 in Colo205 simple cell line (Tn-positive) were analyzed by Western blot using Remab6. FIG. 5B: The numbers of Tn-positive glycoproteins were identified by LC/ESI-MS/MS analysis using immunoprecipitates with ReBaGs6 in the Colo205 simple cell line (Tn-positive).

FIGS. 6A-6C show characterization of mouse ascites BaGs6. FIG. 6A: the binding profiles of ascites (a) and VVA lectin (b) with LS174T and MDA-MB-231 cells by flow cytometry. FIG. 6B: an inhibition assay with pre-incubation of LSC cells with GalNAc, lactose, and Asialo-BSM with ascites (a) and VVA (b). FIG. 6C: cell extracts probed with ascites (a), VVA (b), and anti-actin antibody (c) by western blot.

FIGS. 7A-7C show generation of STn and T glycopeptide arrays. FIG. 7A: The STn glycopeptide array was probed with Alexa488-streptavidin (a). Mass spectrometric analyses of ID6 (b) and ID7 (c) before and after enzyme reactions are shown. FIG. 7B: The T glycopeptide array was probed with biotinylated PNA and Alexa488-streptavidin (a). Mass spectrometric analyses of ID6 (b) and ID7 (c) before and after enzyme reactions. FIG. 7C: Remab6 binding to the CFG glycan microarray. Remab6 tested at 20 μg/ml (top) and 2 μg/ml (bottom). RFU=relative fluorescence units.

FIGS. 8A-8C show LC-ESI-MS/MS analysis using Asialo-BSM. FIG. 8A: Asialo-BSM was analyzed on a Fusion Lumos at two different higher collision dissociation energies (HCD, 25 and 32 eV) to optimize the peptide spectral matches. The precursor monoisotopic patterns for BSM peptides were observed in the top trace for the two different HCD, and matched between the experimental and the theoretical data as a histogram. FIG. 8B: the bottom trace of the profile showing the product ion data for the identified monoisotopic peak for BSM identification. FIG. 8C: the left bar corresponds to the experimental precursor isotope match, and the right bar corresponds to the theoretical data.

FIG. 9 shows immunohistochemical staining in IEC-Cosmc KO mice with Remab6-HRP and Remab6-Fab-HRP. Small intestine-colon-rectum sections in Villi-specific Cosmc KO mice were stained with Remab6-HRP (top) and Remab6-Fab-HRP (bottom). Brown indicates Tn staining, and blue indicates nuclear staining. Scale bar represents 100 μm.

FIGS. 10A-10C show generation of FX knockout HEK293FS cells (FXKO) and production of afucosylated Remab6 (Remab6-FXKO). FXKO cells were established in CRISPR/Cas-9 system as described in Example 2. FIG. 10A-10B: deficient fucosylated glycans on cell surface were observed by flowcytometry with AAL lectin, and N-glycan profile. FIG. 10C: the isolated recombinant Remab6 IgG1 antibody produced by the Remab6-FXKO cells was analyzed by MALDI-TOF mass spectrometry.

FIGS. 11A-11C show uptake of endogenous L-fucose and refucosylation on Remab6 produced recombinant in FXKO cells. FIG. 11A: the pathway for producing GDP-Fuc in cells, and the FX enzyme targeted for deletion in the research is indicated in the figure. Through this pathway the ‘salvage’ pathway for generating GDP-Fuc from L-fucose is indicated. FIG. 11B: the degree of fucosylation by flowcytometry with AAL lectin staining in FXKO cells cultured with L-fucose in defined media for 4 days. FIG. 11C: N-glycans on the recombinant Remab6-FXKO antibody produced by the cells under such conditions, which were analyzed by MALDI-TOF mass spectrometry, and the degree of fucosylation ranged from 9.1% to 91% using 1 to 10 micromolar L-fucose, respectively.

FIG. 12 shows anti-tumor efficacy in Colo205 cell line. The complement-dependent cytotoxicity (CDC) assay was performed with Remab6-WT and Remab6-FXKO antibody in the presence of human serum that naturally contains complement. The antibody-dependent cellular cytotoxicity (ADCC) activity assay was performed with Remab6-WT and Remab6-FXKO antibody in presence of NK cells, using Colo205 cells that are wild-type (WT) or Colo205 cells that express the target antigen known at Tn antigen recognized by the Remab6 antibody. A schematic of an IgG1 recombinant antibody with and without fucose is shown on the upper left.

The accompanying drawings, which are incorporated in and constitute a part of this specification, illustrate certain embodiments, and together with the written description, serve to provide non-limiting examples of certain aspects of the compositions and methods disclosed herein.

### DETAILED DESCRIPTION

The present disclosure is based, at least in part, on the development of anti-Tn antibodies (e.g., BaGs6 and/or Remab6), which possess unexpected superior features compared with known anti-Tn antibodies. Such superior features include at least the following: (i) these antibodies specifically recognize Tn antigen on tumor cells; and/or (ii) these antibodies do not cross-react with Tn antigen on circulating human IgA1, or normal glycans terminating in GalNAc.

Accordingly, provided herein are antibodies capable of binding Tn antigen, as well as nucleic acids encoding said antibodies, and uses thereof for both therapeutic and diagnostic purposes. Also provided herein are kits for use of the antibodies, as well as methods for producing anti-Tn antibodies. In addition, the present disclosure provides chimeric antigen receptors comprising extracellular antigen binding domains derived from any of the anti-Tn antibodies described herein. Also provided in the present disclosure is an engineered cell capable of producing fucosylated antibodies dependent on the presence of fucose in the cell culture media, and methods of producing afucosylated antibodies using the engineered cells described herein. It is within the scope of the present disclosure that any antibody can be produced by the engineered cell as described herein

I. Antibodies that Binds to Tn Antigen

The present disclosure provides antibodies that bind to Tn antigen, for example, Tn antigen on tumor cells.

Tn antigen, as used herein, refers to a carbohydrate antigen defined as two or more N-acetylgalactosamine (GalNAc) monosaccharides in an O-glycosidic alpha-linkage individually linked to the amino acids threonine, serine or tyrosine in close proximity, i.e., within 3 residues of each other (GalNAcα1-O-Ser/Thr). The Tn antigen is a tumor-associated carbohydrate antigen that is not normally expressed in peripheral tissues or blood cells. Expression of this antigen, which is found in a majority of human carcinomas of all types, arises from a blockage in the normal O-glycosylation pathway in which glycans are extended from the common precursor GalNAcα1-O-Ser/Thr (Tn antigen). Under normal conditions, the precursor GalNAcα1-O-Ser/Thr is extended and further modified to normal O-glycans by T-synthase (C1GALT1) to form core 1 O-glycans also called the T or TF antigen. Biosynthesis of active T-synthase requires its molecular chaperone, Cosmc (also termed as C1GAlT1C1). Conditions that cause genetic and epigenetic silencing of T-synthase and/or Cosmc (e.g., cancer) can lead to expression of the precursor Tn antigen. The sialyl-Tn antigen (Neu5Aca2-6GalNAcα-O-Ser/Thr), known as sTn, is a truncated O-glycan containing a sialic acid α-2,6 linked to GalNAc α-O-Serine/Threonine (Ser/Thr). Both Tn and sTn antigens have been reported to be biomarkers of various cancers and are associated with an adverse outcome and poor prognosis in cancer patients. A major obstacle in detecting Tn antigen in circulating glycoproteins is that Tn antigen can also be found on a subset of glycoforms of human IgA1, within the hinge region. Another complication is that anti-Tn reagents that bind terminal α-linked GalNAc residues, as found in blood group A (BGA) and the Forssman-related antigens, may interfere with specific detection of Tn antigen on tumor-derived glycoproteins. Thus, a useful anti-Tn antibody for cancer diagnostics, research, and therapeutics would specifically recognize abnormal Tn antigen, and not cross-react with Tn on IgA1, or normal glycans terminating in GalNAc.

An antibody (interchangeably used in plural form) is an immunoglobulin molecule capable of specific binding to a target antigen (e.g., Tn antigen in the present disclosure), through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The term “isolated antibody” used herein refers to an antibody substantially free from naturally associated molecules, i.e., the naturally associated molecules constituting at most 20% by dry weight of a preparation containing the antibody. Purity can be measured by any appropriate method, e.g., column chromatography, polyacrylamide gel electrophoresis, and HPLC.

A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information system® www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). ee also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made. The antibodies described herein can be murine, rat, human, primate, porcine, or any other origin (including chimeric or humanized antibodies). Such antibodies are non-naturally occurring, i.e., would not be produced in an animal without human act (e.g., immunizing such an animal with a desired antigen or fragment thereof).

In some embodiments, the antibody described herein is a recombinant antibody. A recombinant antibody is an antibody fragments produced by using recombinant antibody coding nucleic acids. The recombinant antibodies described herein can be a full-length antibody, a single chain variable fragment (scFv), or heavy and light chain variable domains of the antibody. In some embodiments, the recombinant antibody is a mouse IgM.

In some embodiments, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the antibody used in the methods described herein is a humanized antibody. Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In some embodiments, the anti-Tn antibodies described herein specifically bind to the corresponding target antigen (e.g., Tn on tumor cells) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (e.g., Tn antigen) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (e.g., binding not detectable in a conventional assay).

In some embodiments, the anti-Tn antibody described herein binds the same epitope of the Tn antigen as a reference antibody disclosed herein (e.g., BaGs6) or competes with the reference antibody for binding to the antigen (e.g., Tn antigen). An “epitope” refers to the site on a target compound that is bound by an antibody, such as a Fab or full-length antibody. Non-limiting examples of epitopes are carbohydrates, peptides, or oligonucleotides. In some embodiments, the epitope is a carbohydrate (e.g., Tn antigen). An epitope can be linear. Alternatively, the epitope can be conformational. An antibody that binds the same epitope as a reference antibody described herein may bind to exactly the same epitope or a substantially overlapping epitope as the reference antibody. Whether two antibodies compete against each other from binding to the cognate antigen can be determined by a competition assay, which is well known in the art. Such antibodies can be identified as known to those skilled in the art, e.g., those having substantially similar structural features (e.g., complementary determining regions), and/or those identified by assays known in the art. For example, competition assays can be performed using one of the reference antibodies to determine whether a candidate antibody binds to the same epitope as the reference antibody or competes against its binding to the Tn antigen.

In some embodiments, the antibodies described herein binds to certain Tn antigen but not the others. In some embodiments, the antibodies described herein specifically binds to Tn antigen on a specific cell (e.g., cancer cell) as relative to Tn antigen from other sauces (e.g., circulating human IgA1) or normal glycans terminating in GalNAc. For example, the antibodies described herein may specifically binds to Tn antigen expressed on cancer cells but not Tn antigen on circulating human IgA1. In some embodiments, the antibodies described herein do not bind to normal glycans terminating in GalNAc (e.g., blood group A (BGA) and the Forssman-related antigens). In other embodiments, Tn antigen is a pan cancer cell marker, and is virtually expressed in all cancer types. In some embodiments, an anti-Tn antibody as described herein has a suitable binding affinity for the target antigen (e.g., Tn antigen on tumor cells) or antigenic epitopes thereof. The specificity of the anti-Tn antibodies described herein to Tn antigens expressed on cancer cells enables them to be suitable for in vivo and in vitro detection of cancer-associated Tn antigen and cancer treatment.

Preferably, the anti-Tn antibodies described herein have a lower affinity for the Tn antigen of normal origin (e.g., Tn antigen on circulating human IgA1), compared with their affinity for Tn antigen associated with cancer. It is preferred that the higher affinity of the anti-Tn antibodies described herein for the cancer Tn-antigen is at least 10%, at least 20%, at least 50%, at least 60%, at least 70%, at least 80%, at least 90%, at least 95% or at least 100 or more compared with the affinity of the anti-Tn antibodies described herein for the Tn-antigen of normal origin (e.g., Tn antigen on circulating human IgA1). It is preferred that the decrease in affinity of the anti-Tn antibodies described herein for Tn-antigen of normal origin (e.g., Tn antigen on circulating human IgA1) is at least 10%, at least 20%, at least 50%, at least 60%, at least 70%, at least 80%, at least 90%, at least 95% or at least 100%, compared with the affinity of the anti-Tn antibodies described herein for the cancer Tn antigen. It is preferred that the increase in affinity of the anti-Tn antibodies described herein for the Tn antigen on cancer cells is at least 1.5-fold, at least 2-fold, at least 5-fold, at least 10-fold, at least 100-fold, more preferably at least 1000-fold greater than the affinity of the anti-Tn antibodies described herein for the Tn-antigen of normal origin (e.g., Tn antigen on circulating human IgA1).

As used herein, “binding affinity” refers to the apparent association constant or K_{A}, which is the ratio of association and dissociation constants, K-on and K-off, respectively. The KA is the reciprocal of the dissociation constant (K_{D}). The anti-Tn antibody described herein may have a binding affinity (K_{D}) of at least 10⁻⁸, 10⁻⁹, 10⁻¹⁰ M, 10⁻¹¹M, or lower for the target antigen or antigenic epitope. For example, the anti-Tn antibody may have a binding affinity of 10⁻⁷ M, 10⁻⁸ M, 10⁻⁹ M, 10⁻¹⁰ M, or lower to Tn antigen. In some embodiments, the anti-Tn antigen has a binding affinity to Tn antigen between 10⁻⁹ M to 10⁻⁶ M. In some embodiments, the anti-Tn antigen has a binding affinity to Tn antigen between 5×10⁻⁹ M to 5×10⁻⁷ M. An increased binding affinity corresponds to a decreased value of K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In some embodiments, the anti-Tn antibodies described herein have a higher binding affinity (a higher K_{A} or smaller K_{D}) to Tn antigen as compared to the binding affinity to carbohydrates (e.g., T or TF antigen). Differences in binding affinity (e.g., for specificity or other comparisons) can be at least 1.5, 2, 2.5, 3, 4, 5, 10, 15, 20, 30, 40, 50, 60, 70, 80, 90, 100, 500, 1,000, 5,000, 10,000, or 10⁵ fold. In some embodiments, any of the anti-Tn antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance (SPR), florescent activated cell sorting (FACS) or spectroscopy (e.g., using a fluorescence assay). Exemplary conditions for evaluating binding affinity are in HBS-P buffer (10 mM HEPES pH 7.4, 150 mM NaCl, 0.005% (v/v) surfactant P20) and PBS buffer (10 mM PO₄⁻³, 137 mM NaCl, and 2.7 mM KCl). These techniques can be used to measure the concentration of bound proteins as a function of target protein concentration. The concentration of bound protein ([Bound]) is generally related to the concentration of free target protein ([Free]) by the following equation:

[Bound]=[Free]/(K_{d}+[Free])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Provided below (Table 5) is an exemplary anti-Tn antibody BaGs6, including its heavy chain and light chain CDR sequences (by IMGT definition) and heavy chain and light chain variable domain sequences.

TABLE 5
Heavy chain and light chain CDR sequences
of exemplary anti-Tn antibody BaGs6

| BaGs6 | CDR1 | CDR2 | CDR3 |
|---|---|---|---|
| Heavy | GYTFTDHAIH | YVSPGNGDIKYNEK | SYGSFDY |
| chain | (SEQ ID | FKG | (SEQ ID |
| | NO: 1) | (SEQ ID NO: 2) | NO: 3) |
| Light | SASSSVSYVY | RTSNLAS | QQYHSYPFT |
| chain | (SEQ ID | (SEQ ID NO: 5) | (SEQ ID |
| | NO: 4) | | NO: 6) |

Heavy chain variable domain sequence of BaGs6
(CDRs in boldface):
(SEQ ID NO: 7)
DTMLQQSDAELVKPGASVKMSCKASGYTFTDHAIHWVKQKPEQGLEWIG
YVSPGNGDIKYNEKFKGKATLSADKSSSTAYMQLNSLTSEDSAVYFCKR
SYGSFDYWGQGTTLTVSS
Light chain variable domain of BaGs6 (CDRs in
boldface):
(SEQ ID NO: 8)
QIVLTQSPAIMSSSPGEKVALSCSASSSVSYVYWYQQKPGSSPKPWIYR
TSNLASGVPARFSGSGSGTSYSLTISSMEAEDAATYYCQQYHSYPFTFG
SGTKLEIKR

In some embodiments, an isolated anti-Tn antibody disclosed herein may comprise the same regions/residues responsible for antigen-binding as a reference antibody (e.g., BaGs6), such as the same specificity-determining residues (SDRs) in the CDRs or the whole CDRs. The regions/residues that are responsible for antigen-binding can be identified from amino acid sequences of the heavy chain/light chain sequences of the reference antibody by methods known in the art. See, e.g., www.bioinf.org.uk/abs; Almagro, J. Mol. Recognit. 17:132-143 (2004); Chothia et al., J. Mol. Biol. 227:799-817 (1987), as well as others known in the art or disclosed herein. In some embodiments, the anti-Tn antibodies disclosed herein have the same VH and/or VL as a reference antibody, such as BaGs6. In some embodiments, the anti-Tn antibodies disclosed herein have the same heavy chain CDRs and/or light chain CDRs as a reference antibody, such as BaGs6.

In some embodiments, the isolated anti-Tn antibody comprises a heavy chain variable region that comprises a heavy chain CDR1 (HC CDR1), a heavy chain CDR2 (HC CDR2), and a heavy chain CDR3 (HC CDR3).

In some embodiments, following the IMGT definition, the HC CDR1 comprises the amino acid sequence as set forth in SEQ ID NO: 1. Alternatively or in addition, the HC CDR2 comprises the amino acid sequence as set forth in SEQ ID NO: 2. Alternative or in addition, the HC CDR3 comprises the amino acid sequence as set forth in SEQ ID NO: 3.

Alternatively or in addition, the isolated anti-Tn antibody comprises a light chain variable region that comprises a light chain CDR1 (LC CDR1), a light chain CDR2 (LC CDR2), and a light chain CDR3 (LC CDR3).

In some embodiments, following the IMGT definition, the LC CDR1 comprises the amino acid sequence as set forth in SEQ ID NO: 4. Alternatively or in addition, the LC CDR2 comprises the amino acid sequence as set forth in SEQ ID NO: 5. Alternative or in addition, the LC CDR3 comprises the amino acid sequence as set forth in SEQ ID NO: 6.

The CDRs of an antibody may have different amino acid sequences when different definition systems are used (e.g., the IMGT definition, the Kabat definition, or the Chothia definition). A definition system annotates each amino acid in a given antibody sequence (e.g., VH or VL sequence) with a number, and numbers corresponding to the heavy chain and light chain CDRs are provided in Table 6. The CDRs listed in Table 5 are defined in accordance with the IMGT definition. One skilled in the art is able to derive the CDR sequences using the different numbering systems for the anti-Tn antibodies provided in Table 5.

TABLE 6
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M. -P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

Also, within the scope of the present disclosure are functional variants of any of the exemplary anti-Tn antibodies as disclosed herein. A functional variant may contain one or more amino acid residue variations in the VH and/or VL, or in one or more of the HC CDRs and/or one or more of the LC CDRs as relative to the reference antibody (e.g., BaGs6), while retaining substantially similar binding and biological activities (e.g., substantially similar binding affinity, binding specificity, anti-tumor activity, or a combination thereof) as the reference antibody.

In some embodiments, the anti-Tn antibody of the present disclosure comprises a HC CDR1, HC CDR2, and HC CDR3 of a heavy chain variable domain having the amino acid sequence of SEQ ID NO: 7. Alternatively or in addition, the anti-Tn antibody of the present disclosure comprises a LC CDR1, LC CDR2, and LC CDR3 of a light chain variable domain having the amino acid sequence of SEQ ID NO: 8.

In some embodiments, according to the IMGT definition system, the anti-Tn antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6.

In some embodiments, anti-Tn antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-Tn antibody of the present disclosure comprises a LC CDR1, a LC CDR2, and a LC CDR3, which collectively contains no more than 5 amino acid variations (e.g., no more than 5, 4, 3, 2, or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, the anti-Tn antibody of the present disclosure comprises a HC CDR1, a HC CDR2, and a HC CDR3 that collectively are at least 80% (e.g., 80%, 85%, 90%, 95%, 98%, 99%, or 100%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-Tn antibody of the present disclosure comprises a LC CDR1, a LC CDR2, and a LC CDR3 that collectively are at least 80% (e.g., 80%, 85%, 90%, 95%, 98%, 99%, or 100%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, the anti-Tn antibody of the present disclosure comprises a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-Tn antibody of the present disclosure comprises a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, the anti-Tn antibody of the present disclosure comprises a VH of the amino acid sequence of SEQ ID NO: 7. Alternatively or in addition, the anti-Tn antibody of the present disclosure comprises a VL of the amino acid sequence of SEQ ID NO: 8.

In some embodiments, the anti-Tn antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-Tn antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8.

In some embodiments, the anti-Tn antibody of the present disclosure comprises a VH comprising an amino acid sequence that is at least 80% (e.g., 80%, 85%, 90%, 95%, 98%, 99%, or 100%) identical to the VH as set forth in SEQ ID NO: 7. Alternatively or in addition, the anti-Tn antibody of the present disclosure comprises a VL comprising an amino acid sequence that is at least 80% (e.g., 80%, 85%, 90%, 95%, 98%, 99%, or 100%) identical to the VL as set forth in SEQ ID NO: 8. In some embodiments, the isolated anti-Tn antibody disclosed herein comprises a HC CDR1, a HCCDR2, or a HC CDR3, each contains no more than 5 amino acid variations (e.g., no more than 5, 4, 3, 2, 1 amino acid variation) as compared with the HC CDR1, HC CDR2, and HC CDR3 of a reference antibody such as BaGs6. In some examples, the anti-Tn antibody disclosed herein may comprise a HC CDR1, a HC CDR2, and a HC CDR3, each of which contains no more than 5 amino acid variations (e.g., no more than 4, 3, 2, or 1 amino acid variation) as the counterpart HC CDR of a reference antibody such as BaGs6.

Alternatively or in addition, the isolated anti-Tn antibody may comprise a LC CDR1, a LC CDR2, and a LC CDR3, each contains no more than 10 amino acid variations (e.g., no more than 9, 8, 7, 6, 5, 4, 3, 2 or 1 amino acid variation) as compared with the LC CDR1, LC CDR2, and LC CDR3 of the reference antibody (e.g., BaGs6). In some examples, an anti-Tn antibody may comprise a LC CDR1, a LC CDR2, and a LC CDR3, each of which contains no more than 5 amino acid variations (e.g., no more than 4, 3, 2, or 1 amino acid variation) as the counterpart LC CDR of the reference antibody (e.g., BaGs6). In some embodiment, the antibody comprises a LC CDR3, which contains no more than 5 amino acid variations (e.g., no more than 4, 3, 2, or 1 amino acid variation) as the LC CDR3 of the reference antibody (e.g., BaGs6).

In some embodiments, the isolated anti-Tn antibody disclosed herein comprises a HC CDR1, a HCCDR2, and a HC CDR3, which collectively contains no more than 10 amino acid variations (e.g., no more than 9, 8, 7, 6, 5, 4, 3, 2, or 1 amino acid variation) as compared with the HC CDR1, HC CDR2, and HC CDR3 of a reference antibody such as BaGs6. “Collectively” means that the total number of amino acid variations in all of the three HC CDRs is within the defined range. In some examples, the anti-Tn antibody disclosed herein may comprise a HC CDR1, a HC CDR2, and a HC CDR3, at least one of which contains no more than 5 amino acid variations (e.g., no more than 4, 3, 2, or 1 amino acid variation) as the counterpart HC CDR of a reference antibody such as BaGs6.

Alternatively or in addition, the isolated anti-Tn antibody may comprise a LC CDR1, a LC CDR2, and a LC CDR3, which collectively contains no more than 10 amino acid variations (e.g., no more than 9, 8, 7, 6, 5, 4, 3, 2 or 1 amino acid variation) as compared with the LC CDR1, LC CDR2, and LC CDR3 of the reference antibody (e.g., BaGs6). In some examples, an anti-Tn antibody may comprise a LC CDR1, a LC CDR2, and a LC CDR3, at least one of which contains no more than 5 amino acid variations (e.g., no more than 4, 3, 2, or 1 amino acid variation) as the counterpart LC CDR of the reference antibody (e.g., BaGs6). In some embodiment, the antibody comprises a LC CDR3, which contains no more than 5 amino acid variations (e.g., no more than 4, 3, 2, or 1 amino acid variation) as the LC CDR3 of the reference antibody (e.g., BaGs6).

In some embodiments, the isolated anti-Tn antibody disclosed herein may comprise heavy chain CDRs that collectively are at least 80%, at least 85%, at least 90%, at least 91%, at least 92%, at least 93%, at least 94%, at least 95%, at least 96%, at least 97%, at least 98%, at least 99%, or 100% identical to the heavy chain CDRs of a reference antibody such as BaGs6. Alternatively or in addition, the isolated anti-Tn antibody may comprise light chain CDRs that collectively are at least 80%, at least 85%, at least 90%, at least 91%, at least 92%, at least 93%, at least 94%, at least 95%, at least 96%, at least 97%, at least 98%, at least 99%, or 100% identical to the light chain CDRs of the reference antibody, such as BaGs6. In some embodiments, the anti-Tn antibody may comprise a heavy chain variable region that is at least 80%, at least 85%, at least 90%, at least 91%, at least 92%, at least 93%, at least 94%, at least 95%, at least 96%, at least 97%, at least 98%, at least 99%, or 100% identical to the heavy chain variable region of a reference antibody such as BaGs6 and/or a light chain variable region that is at least 80%, at least 85%, at least 90%, at least 91%, at least 92%, at least 93%, at least 94%, at least 95%, at least 96%, at least 97%, at least 98%, at least 99%, or 100% identical to the light chain variable region of the reference antibody such as BaGs6. In some embodiments, the anti-Tn antibody may comprise a heavy chain variable region that is at least 80%, at least 85%, at least 90%, at least 91%, at least 92%, at least 93%, at least 94%, at least 95%, at least 96%, at least 97%, at least 98%, at least 99%, or 100% identical to the heavy chain variable region as set forth in SEQ ID NO: 7 and/or a light chain variable region that is at least 80%, at least 85%, at least 90%, at least 91%, at least 92%, at least 93%, at least 94%, at least 95%, at least 96%, at least 97%, at least 98%, at least 99%, or 100% identical to the light chain variable region as set forth in SEQ ID NO: 8.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the anti-Tn antibodies may include modifications to improve properties of the antibody, for example, stability, oxidation, isomerization and deamidation. In some instances, the antibody may comprise residues that are not found in the framework (FR region) sequences of the reference antibody (e.g., BaGs6) because certain amino acids are substituted for improved properties. In some instances, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. In some instances, conservative substitutions of amino acids may include substitutions made amongst amino acids within the following groups: (a) E, D; (b) M, I, L, V; (c) F, Y, W; (d) K, R, H; (e) A, G; (f) S, T; and (g) Q, N.

In some embodiments, the heavy chain of any of the anti-Tn antibodies as described herein may further comprise a heavy chain constant region (5 CH) or a portion thereof (e.g., CH1, CH2, CH3, or a combination thereof). The heavy chain constant region can of any suitable origin, e.g., human, mouse, rat, or rabbit. In some embodiments, the heavy chain constant region is from a mouse IgM. In some embodiments, the heavy chain constant region is from a human IgG (a gamma heavy chain), e.g., IgG1, IgG2, or IgG4. In one example, the heavy chain constant region is of subclass IgG1.

The light chain of any of the anti-Tn antibodies described herein may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

In some embodiments, the isolated anti-Tn antibodies described herein are full length antibodies. In some embodiments, the isolated anti-Tn antibody is a recombinant mouse IgM.

An exemplary heavy chain amino acid sequence of a recombinant anti-Tn mouse IgM (ReGs6) is set forth in SEQ ID NO: 9 (VH underlined, CDRs in bold face):

(SEQ ID NO: 9)
DTMLQQSDAELVKPGASVKMSCKASGYTFTDHAIHWVKQKPEQGLEWIG
YVSPGNGDIKYNEKFKGKATLSADKSSSTAYMQLNSLTSEDSAVYFCKR
SYGSFDYWGQGTTLTVSSESQSFPNVFPLVSCESPLSDKNLVAMGCLAR
DFLPSTISFTWNYQNNTEVIQGIRIFPTLRTGGKYLATSQVLLSPKSIL
EGSDEYLVCKIHYGGKNRDLHVPIPAVAEMNPNVNVFVPPRDGFSGPAP
RKSKLICEATNFTPKPITVSWLKDGKLVESGFTTDPVTIENKGSTPQTY
KVISTLTISEIDWLNLNVYTCRVDHRGLTFLKNVSSTCAASPSTDILTF
TIPPSFADIFLSKSANLTCLVSNLATYETLNISWASQSGEPLETKIKIM
ESHPNGTFSAKGVASVCVEDWNNRKEFVCTVTHRDLPSPQKKFISKPNE
VHKHPPAVYLLPPAREQLNLRESATVTCLVKGFSPADISVQWLQRGQLL
PQEKYVTSAPMPEPGAPGFYFTHSILTVTEEEWNSGETYTCVVGHEALP
HLVTERTVDKSIGKPTLYNVSLIMSDTGGTCY

In some embodiments, the mouse IgM anti-Tn antibody comprises a mouse K light chain. An exemplary κ light chain amino acid sequence of a recombinant anti-Tn mouse IgM is set forth in SEQ ID NO: 10 (VL underlined, CDRs in bold face):

(SEQ ID NO: 10)
QIVLTQSPAIMSSSPGEKVALSCSASSSVSYVYWYQQKPGSSPKPWIYR
TSNLASGVPARFSGSGSGTSYSLTISSMEAEDAATYYCQQYHSYPFTFG
SGTKLEIKRADAAPTVSIFPPSSEQLTSGGASVVCFLNNFYPKDINVKW
KIDGSERQNGVLNSWTDQDSKDSTYSMSSTLTLTKDEYERHNSYTCEAT
HKTSTSPIVKSFNRNEC

In some embodiments, the isolated anti-Tn antibody described herein comprises a heavy chain that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the heavy chain variable region as set forth in SEQ ID NO: 9. Alternatively or in addition, the isolated anti-Tn antibody described herein comprises a light chain that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the heavy chain variable region as set forth in SEQ ID NO: 9.

In some embodiments, the isolated anti-Tn antibodies described herein are full length antibodies. In some embodiments, the isolated anti-Tn antibody is a chimeric antibody. In some embodiments, the anti-Tn antibody comprises a VH and/or VL from mouse, and the constant domain from human. In some embodiments, the chimeric antibody comprises a heavy chain constant domain of human IgG. In some embodiments, the chimeric antibody comprises a heavy chain constant domain of human IgG₁ (Remab).

An exemplary heavy chain amino acid sequence of a chimeric anti-Tn antibody (Remab) is set forth in SEQ ID NO: 11 (VH underlined, CDRs in bold face):

(SEQ ID NO: 11)
DTMLQQSDAELVKPGASVKMSCKASGYTFTDHAIHWVKQKPEQGLEWIG
YVSPGNGDIKYNEKFKGKATLSADKSSSTAYMQLNSLTSEDSAVYFCKR
SYGSFDYWGQGTTLTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLS
LSPGK

In some embodiments, the chimeric anti-Tn antibody comprises a human κ light chain. An exemplary κ light chain amino acid sequence of a chimeric anti-Tn antibody is set forth in SEQ ID NO: 12 (VL underlined, CDRs in bold face):

(SEQ ID NO: 12)
QIVLTQSPAIMSSSPGEKVALSCSASSSVSYVYWYQQKPGSSPKPWIYR
TSNLASGVPARFSGSGSGTSYSLTISSMEAEDAATYYCQQYHSYPFTFG
SGTKLEIKRTVAAPSVFIFPPSDEQLKSGTASVVCLLNNFYPREAKVQW
KVDNALQSGNSQESVTEQDSKDSTYSLSSTLILSKADYEKHKVYACEVT
HQGLSSPVTKSFNRGEC

In some embodiments, the isolated anti-Tn antibody described herein comprises a heavy chain that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the heavy chain variable region as set forth in SEQ ID NO: 11. Alternatively or in addition, the isolated anti-Tn antibody described herein comprises a light chain that is at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the heavy chain variable region as set forth in SEQ ID NO: 12.

As described herein, the anti-Tn antibody can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies.

Also, with the scope of the present disclosure are antibody conjugates comprising the anti-Tn antibody described herein and another agent. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. In some embodiments, the anti-Tn antibody can be conjugated to a detectable label (e.g., fluorescent proteins or agents, colorimetric agents, luminescent agents, etc), an imaging agent, a therapeutic agent (e.g., cytotoxic agent or toxins) or oligonucleotides. In some embodiments, the imaging agent is a radioactive agent selected from the group consisting of fluorine-18, zirconium-89, copper-64, yttrium-86, indium-111, ¹²²I, ¹²³I, ¹²⁴I, ¹²⁵I, ¹³¹I, ¹⁸F, ⁷⁵Br, ⁷⁶Br, ⁷⁷Br, ²¹¹At ²²⁵Ac, ¹⁷⁷Lu ¹⁵³Sm, ¹⁸⁶Re, ¹⁸⁸Re, ⁶⁷Cu, ²¹³Bi, ²¹²Bi, ²¹²Pb, and ⁶⁷Ga. Exemplary radiopharmaceuticals suitable for in vivo imaging include ¹¹¹In Oxyquinoline, ¹³¹I Sodium iodide, ^{99m}Tc Mebrofenin, and ^{99m}Tc Red Blood Cells, ¹²³I Sodium iodide, ^{99m}Tc Exametazime, ^{99m}Tc Macroaggregate Albumin, ^{99m}Tc Medronate, ^{99m}Tc Mertiatide, ^{99m}Tc Oxidronate, ^{99m}Tc Pentetate, ^{99m}Tc Pertechnetate, ^{99m}Tc Sestamibi, ^{99m}Tc Sulfur Colloid, ^{99m}Tc Tetrofosmin, Thallium-201, or Xenon-133. In some embodiments, the anti-Tn antibody is conjugated to a cytotoxic agent selected from dolastin 10, zogamicin, monomethyl auristatin E (MMAE), cryptophycin and analogs thereof, enediyne antiobiotics including wesperamicin and maytansines (emtasine DM1/DM4), calicheamicin, capecitabine, lapatinib, anthracyclines, duocarmycins, or pyyrolobenzodiazepines. In some embodiments, the anti-Tn antibody is conjugated to a toxin, such as Pseudomonas exotoxin or diphtheria toxin. In some embodiments, the anti-Tn antibody is conjugated to a fluorescent protein or a fluorescent compound. The fluorescent protein includes, but is not limited to, wt-GFP, green fluorescent protein (e.g., EGFP, Emerald, Superfolder GFP, Azami Green, mWasabi, TagGFP, TurboGFP, AcGFP, ZsGreen, T-Sapphire, etc.), blue fluorescent protein, (e.g., EBFP, EBFP2, Azurite, mTagBFP, etc.), cyan fluorescent protein (e.g., ECFP, mECFP, Cerulean, mTurquoise, CyPet, AmCyanl, Midori-Ishi Cyan, TagCFP, mTFP1 (Teal), etc.), yellow fluorescent protein (e.g., EYFP, Topaz, Venus, mCitrine, YPet, TagYFP, PhiYFP, ZsYellowl, mBanana, etc.), orange fluorescent protein (e.g., Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, etc.), or red fluorescent protein (e.g., mRuby, mApple, mStrawberry, AsRed2, mRFP1, JRed, mCherry, HcRedl, mRaspberry, dKeima-Tandem, HcRed-Tandem, mPlum, AQ143, etc.)

### II. Preparation of Anti-Tn Antibodies

Antibodies capable of binding Tn antigen (e.g., Tn antigen specifically on cancer cells) as described herein can be made by any method known in the art. See, for example, Harlow and Lane, (1998) Antibodies: A Laboratory Manual, Cold Spring Harbor Laboratory, New York.

In some embodiments, antibodies specific to a target antigen (e.g., Tn antigen) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

If desired, the polynucleotide sequence of the antibody (e.g., anti-Tn antibody) may be cloned into a vector for expression or propagation. The sequence encoding the antibody of interest may be maintained in vector in a host cell, and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in detecting Tn antigen. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are XenomouseR™ from Amgen, Inc. (Fremont, CA) and HuMAb-MouseR™ and TC Mouse™ from Medarex, Inc. (Princeton, NJ) or H2L2 mice from Harbour Antibodies BV (Holland). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, human HEK293 cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

Alternatively, techniques described for the production of single chain antibodies (U.S. Pat. Nos. 4,946,778 and 4,704,692) can be adapted to produce a phage or yeast scFv library and scFv clones specific to Tn antigen can be identified from the library following routine procedures. Positive clones can be subjected to further screening to identify those that binds to Tn-antigen on tumor cells.

Antibodies obtained following a method known in the art and described herein can be characterized using methods well known in the art. For example, one method is to identify the epitope to which the antigen binds, or “epitope mapping”. There are many methods known in the art for mapping and characterizing the location of epitopes on antigens, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In one example, epitope mapping can be accomplished use H/D-Ex (hydrogen deuterium exchange) coupled with proteolysis and mass spectrometry. In an additional example, epitope mapping can be used to determine the sequence to which an antibody binds. The epitope can be a linear epitope, or a conformational epitope formed by a three-dimensional interaction of antigen that may not necessarily be contained in a single stretch (primary structure linear sequence). Epitopes of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which the Tn antigen has been replaced (swapped) with carbohydrates antigen from a closely related, but antigenically distinct glycans (such as T or TF antigen). By assessing binding of the antibody to other glucans, the importance of the particular antigen fragment to antibody binding can be assessed. Alternatively, competition assays can be performed using other antibodies known to bind to the same antigen to determine whether an antibody binds to the same epitope as the other antibodies. Competition assays are well known to those of skill in the art.

In some examples, an anti-Tn antibody is prepared by recombinant technology as exemplified below. Nucleic acids encoding the heavy and light chain of an anti-Tn antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

An exemplary nucleic acid encoding the heavy chain of mouse IgM anti-Tn antibody (ReBaGs6) is set forth in SEQ ID NO: 13:

(SEQ ID NO: 13)
GATACAATGCTCCAGCAGTCCGACGCCGAACTGGTTAAGCCTGGCGCCT
CTGTGAAGATGAGCTGCAAGGCCAGCGGCTACACCTTCACAGATCACGC
CATCCACTGGGTCAAGCAGAAGCCTGAACAGGGCCTCGAATGGATCGGC
TACGTTAGCCCTGGCAACGGCGACATCAAGTACAACGAGAAGTTCAAGG
GCAAAGCCACACTGAGCGCCGACAAGAGCAGCAGCACAGCCTACATGCA
GCTGAACAGCCTGACAAGCGAGGACAGCGCCGTGTACTTCTGCAAGAGA
AGCTACGGCAGCTTCGACTACTGGGGCCAGGGCACAACACTGACAGTGT
CTAGCGAGAGTCAGTCCTTCCCAAATGTCTTCCCCCTCGTCTCCTGCGA
GAGCCCCCTGTCTGATAAGAATCTGGTGGCCATGGGCTGCCTGGCCCGG
GACTTCCTGCCCAGCACCATTTCCTTCACCTGGAACTACCAGAACAACA
CTGAAGTCATCCAGGGTATCAGAACCTTCCCAACACTGAGGACAGGGGG
CAAGTACCTAGCCACCTCGCAGGTGTTGCTGTCTCCCAAGAGCATCCTT
GAAGGTTCAGATGAATACCTGGTATGCAAAATCCACTACGGAGGCAAAA
ACAGAGATCTGCATGTGCCCATTCCAGCTGTCGCAGAGATGAACCCCAA
TGTAAATGTGTTCGTCCCACCACGGGATGGCTTCTCTGGCCCTGCACCA
CGCAAGTCTAAACTCATCTGCGAGGCCACGAACTTCACTCCAAAACCGA
TCACAGTATCCTGGCTAAAGGATGGGAAGCTCGTGGAATCTGGCTTCAC
CACAGATCCGGTGACCATCGAGAACAAAGGATCCACACCCCAAACCTAC
AAGGTCATAAGCACACTTACCATCTCTGAAATCGACTGGCTGAACCTGA
ATGTGTACACCTGCCGTGTGGATCACAGGGGTCTCACCTTCTTGAAGAA
CGTGTCCTCCACATGTGCTGCCAGTCCCTCCACAGACATCCTAACCTTC
ACCATCCCCCCCTCCTTTGCCGACATCTTCCTCAGCAAGTCCGCTAACC
TGACCTGTCTGGTCTCAAACCTGGCAACCTATGAAACCCTGAATATCTC
CTGGGCTTCTCAAAGTGGTGAACCACTGGAAACCAAAATTAAAATCATG
GAAAGCCATCCCAATGGCACCTTCAGTGCTAAGGGTGTGGCTAGTGTTT
GTGTGGAAGACTGGAATAACAGGAAGGAATTTGTGTGTACTGTGACTCA
CAGGGATCTGCCTTCACCACAGAAGAAATTCATCTCAAAACCCAATGAG
GTGCACAAACATCCACCTGCTGTGTACCTGCTGCCACCAGCTCGTGAGC
AACTGAACCTGAGGGAGTCAGCCACAGTCACCTGCCTGGTGAAGGGCTT
CTCTCCTGCAGACATCAGTGTGCAGTGGCTTCAGAGAGGGCAACTCTTG
CCCCAAGAGAAGTATGTGACCAGTGCCCCGATGCCAGAGCCTGGGGCCC
CAGGCTTCTACTTTACCCACAGCATCCTGACTGTGACAGAGGAGGAATG
GAACTCCGGAGAGACCTATACCTGTGTTGTAGGCCACGAGGCCCTGCCA
CACCTGGTGACCGAGAGGACCGTGGACAAGTCCACTGGTAAACCCACAC
TGTACAATGTCTCCCTGATCATGTCTGACACAGGCGGCACCTGCTAT

An exemplary nucleic acid encoding the light chain of mouse IgM anti-Tn antibody (ReBaGs6) is set forth in SEQ ID NO: 14:

(SEQ ID NO: 14)
CAGATCGTGCTGACACAGAGCCCCGCCATTATGTCTAGCAGCCCTGGCG
AAAAGGTGGCCCTGAGCTGTTCTGCCAGCAGCAGCGTGTCCTACGTGTA
CTGGTATCAGCAGAAGCCCGGCAGCAGCCCCAAGCCTTGGATCTACAGA
ACAAGCAATCTGGCCAGCGGCGTGCCAGCCAGATTTTCTGGTTCTGGCA
GCGGCACCAGCTACAGCCTGACAATCTCTAGCATGGAAGCCGAGGACGC
CGCCACCTACTACTGTCAGCAGTACCACAGCTACCCCTTCACCTTTGGC
TCCGGCACCAAGCTGGAAATCAAGAGAGCAGATGCTGCACCAACTGTAT
CCATCTTCCCACCATCCAGTGAGCAGTTAACATCTGGAGGTGCCTCAGT
CGTGTGCTTCTTGAACAACTTCTACCCCAAAGACATCAATGTCAAGTGG
AAGATTGATGGCAGTGAACGACAAAATGGCGTCCTGAACAGTTGGACTG
ATCAGGACAGCAAAGACAGCACCTACAGCATGAGCAGCACCCTCACGTT
GACCAAGGACGAGTATGAACGACATAACAGCTATACCTGTGAGGCCACT
CACAAGACATCAACTTCACCCATTGTCAAGAGCTTCAACAGGAATGAGT
GT

An exemplary nucleic acid encoding the heavy chain of chimeric human IgG₁ anti-Tn antibody (Remab) is set forth in SEQ ID NO: 15:

(SEQ ID NO: 15)
GATACAATGCTCCAGCAGTCCGACGCCGAACTGGTTAAGCCTGGCGCCT
CTGTGAAGATGAGCTGCAAGGCCAGCGGCTACACCTTCACAGATCACGC
CATCCACTGGGTCAAGCAGAAGCCTGAACAGGGCCTCGAATGGATCGGC
TACGTTAGCCCTGGCAACGGCGACATCAAGTACAACGAGAAGTTCAAGG
GCAAAGCCACACTGAGCGCCGACAAGAGCAGCAGCACAGCCTACATGCA
GCTGAACAGCCTGACAAGCGAGGACAGCGCCGTGTACTTCTGCAAGAGA
AGCTACGGCAGCTTCGACTACTGGGGCCAGGGCACAACACTGACAGTGT
CTAGCGCCTCTACAAAGGGCCCTAGCGTTTTCCCACTGGCTCCTAGCAG
CAAGAGCACATCTGGTGGAACAGCCGCTCTGGGCTGCCTGGTCAAGGAT
TACTTTCCTGAGCCTGTGACCGTGTCCTGGAATTCTGGTGCTCTGACCA
GCGGCGTGCACACATTTCCAGCCGTGCTGCAGTCTAGCGGCCTGTACTC
TCTGTCTAGCGTGGTCACAGTGCCTAGCTCTAGCCTGGGCACCCAGACC
TACATCTGCAACGTGAACCACAAGCCTAGCAACACCAAGGTGGACAAGA
AGGTGGAACCCAAGAGCTGCGACAAGACCCACACCTGTCCTCCATGTCC
TGCTCCAGAACTGCTCGGCGGACCCTCCGTTTTCCTGTTTCCACCTAAG
CCTAAGGACACCCTGATGATCAGCAGAACCCCTGAAGTGACCTGCGTGG
TGGTGGATGTGTCTCACGAGGACCCCGAAGTGAAGTTCAATTGGTACGT
GGACGGCGTGGAAGTGCACAACGCCAAGACCAAGCCTAGAGAGGAACAG
TACAACAGCACCTACAGAGTGGTGTCCGTGCTGACAGTGCTGCACCAGG
ACTGGCTGAACGGCAAAGAGTACAAGTGCAAGGTGTCCAACAAGGCCCT
GCCTGCTCCTATCGAGAAAACCATCAGCAAGGCCAAGGGCCAGCCAAGA
GAACCCCAGGTTTACACCCTGCCTCCAAGCCGGGAAGAGATGACCAAGA
ATCAGGTGTCCCTGACCTGCCTCGTGAAGGGCTTCTACCCTTCCGATAT
CGCCGTGGAATGGGAGAGCAATGGCCAGCCTGAGAACAACTACAAGACA
ACCCCTCCTGTGCTGGACTCCGATGGCTCATTCTTCCTGTACAGCAAGC
TGACCGTGGACAAGTCCAGATGGCAGCAGGGCAATGTGTTCTCCTGCTC
TGTGATGCACGAAGCCCTGCACAACCACTACACCCAGAAGTCTCTTAGC
CTGTCTCCTGGCAAA

An exemplary nucleic acid encoding the heavy chain of chimeric human IgG₁ anti-Tn antibody (Remab) is set forth in SEQ ID NO: 16:

(SEQ ID NO: 16)
CAGATCGTGCTGACACAGAGCCCCGCCATTATGTCTAGCAGCCCTGGCG
AAAAGGTGGCCCTGAGCTGTTCTGCCAGCAGCAGCGTGTCCTACGTGTA
CTGGTATCAGCAGAAGCCCGGCAGCAGCCCCAAGCCTTGGATCTACAGA
ACAAGCAATCTGGCCAGCGGCGTGCCAGCCAGATTTTCTGGTTCTGGCA
GCGGCACCAGCTACAGCCTGACAATCTCTAGCATGGAAGCCGAGGACGC
CGCCACCTACTACTGTCAGCAGTACCACAGCTACCCCTTCACCTTTGGC
TCCGGCACCAAGCTGGAAATCAAGAGAACAGTGGCCGCTCCGAGCGTGT
TCATCTTTCCACCAAGCGACGAGCAGCTGAAGTCTGGCACAGCCTCTGT
CGTGTGCCTGCTGAACAACTTCTACCCCAGAGAAGCCAAGGTGCAGTGG
AAGGTGGACAATGCCCTGCAGAGCGGCAATAGCCAAGAGAGCGTGACCG
AGCAGGACAGCAAGGACTCTACCTACTCTCTGAGCAGCACCCTGACACT
GAGCAAGGCCGACTACGAGAAGCACAAAGTGTACGCCTGCGAAGTGACA
CACCAGGGCCTGTCTAGCCCTGTGACCAAGAGCTTCAACCGGGGCGAGT
GT

In some embodiments, the present disclosure provides an isolated anti-Tn antibody produced by expressing in a host cell: i) a nucleic acid sequence encoding a heavy chain, wherein the nucleic acid sequence is at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 13; and/or (ii) a nucleic acid sequence encoding a light chain, wherein the nucleic acid sequence is at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 14.

In some embodiments, the present disclosure provides an isolated anti-Tn antibody produced by expressing in a host cell: i) a nucleic acid sequence encoding a heavy chain, wherein the nucleic acid sequence is at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 15; and/or (ii) a nucleic acid sequence encoding a light chain, wherein the nucleic acid sequence is at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 16.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV promoter, and the herpes simplex tk virus promoter.

Regulatable promoters can also be used. Such regulatable promoters include those using the lac repressor from E. coli as a transcription modulator to regulate transcription from lac operator bearing mammalian cell promoters (Brown, M. et al., Cell, 49:603-612 (1987)], those using the tetracycline repressor (tetR) [Gossen, M., and Bujard, H., Proc. Natl. Acad. Sci. USA 89:5547-555115 (1992); Yao, F. et al., Human Gene Therapy, 9:1939-1950 (1998); Shockelt, P., et al., Proc. Natl. Acad. Sci. USA, 92:6522-6526 (1995)). Other systems include FK506 dimer, VP16 or p65 using astradiol, RU486, diphenol murislerone, or rapamycin. Inducible systems are available from Invitrogen, Clontech, and Ariad, among others.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987)); Gossen and Bujard (1992); (M. Gossen et al., Proc. Natl. Acad. Sci. USA, 89:5547-5551(1992)) combined the tetracycline repressor (tetR) with the transcription activator (VP 16) to create a tetR-mammalian cell transcription activator fusion protein, tTa (tetR-VP 16), with the tetO bearing minimal promoter derived from the human cytomegalovirus (hCMV) promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy). One particular advantage of this tetracycline inducible switch is that it does not require the use of a tetracycline repressor-mammalian cells trans-activator or repressor fusion protein, which in some instances can be toxic to cells (Gossen et al., Proc. Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-Tn antibody, as also described herein. The recombinant expression vector can be introduced into a suitable host cell (e.g., a dhFr− CHO cell) by a conventional method, e.g., calcium phosphate mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody. In some embodiments, an exemplary host cell includes, but is not limited to, a HEK293 cell, Chinese hamster ovary (CHO) cell, dhFr− CHO cell, HeLa cell, HT-1080 cell, PER.C6, HKB-11 cell, CAP cell, HuH07 cell, NS0 cell, HKB11, Sp2/0 cell, BHK cell, or C127 cell. In some embodiments, the cell is HEK293 cell.

In some embodiments, two recombinant expression vectors are provided, one encoding the heavy chain of the anti-Tn antibody and the other encoding the light chain of the anti-Tn antibody. Both of the two recombinant expression vectors can be introduced into a suitable host cell (e.g., dhFr− CHO cell) by a conventional method, e.g., calcium phosphate-mediated transfection. Alternatively, each of the expression vectors can be introduced into a suitable host cell. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-Tn antibody as described herein, vectors (e.g., expression vectors) containing such; and host cells comprising the vectors are within the scope of the present disclosure.

### III. Afucosylated Anti-Tn Antibody and Methods for Producing the Same

Antibodies can be glycosylated with an N-glycan at the Fc-gamma glycosylation site in the heavy chain (e.g., typically at Asn297) of the Fc region. Generally, antibodies include two heavy chains and each antibody therefore can have two Fc-gamma N-glycans. A variety of glycosylation patterns have been observed at the Fc gamma glycosylation site and the oligosaccharides found at this site include galactose, N-acetylglucosamine (GlcNac), mannose, sialic acid, N-acetylneuraminic acid (NeuAc or NANA), N-glycolylneuraminic (NGNA) and fucose. N-glycans found at the Fc gamma glycosylation site generally have a common core structure consisting of an unbranched chain of a first N-acetylglucosamine (GlcNAc), which is attached to the asparagine of the antibody, a second GlcNAc that is attached to the first GlcNac and a first mannose that is attached to the second GlcNac. Two additional mannoses are attached to the first mannose of the GlcNAc-GlcNAc-mannose chain to complete the core structure and providing two “arms” for additional glycosylation. In addition, fucose residues may be attached to the N-linked first GlcNAc. In the biosynthetic route to this N-glycan motif, several GlcNAc transferases attach GlcNAc residues to the mannoses of the glycan core, which can be further extended by galactose, sialic acid and fucose residues. This glycosylation motif is called “complex” structure. The glycosylation pattern of the N-glycans can be determined by a variety of methods known in the art. For example, methods of analyzing carbohydrates on proteins have been described in U.S. Patent Applications US 2006/0057638 and US 2006/0127950. The methods of analyzing carbohydrates on proteins are incorporated herein by reference.

L-fucose (6-deoxy-L-galactose) is a monosaccharide that is a common component of many N- and O-linked glycans and glycolipids produced by mammalian cells. Two structural features distinguish fucose from other six-carbon sugars present in mammals. These include the lack of a hydroxyl group on the carbon at the 6-position (C-6) and the L-configuration. Fucose frequently exists as a terminal modification of glycan structures. Specific terminal glycan modifications, including fucosylation, can confer unique functional properties to oligosaccharides and are often regulated during ontogeny and cellular differentiation. Important roles for fucosylated glycans have been demonstrated in a variety of biological settings (Listinsky et al., 1998, Fucose in N-glycans: from plant to man. Biochim. Biophys. Acta, 1473, 216-236; Staudacher et al., 1999, Fucose in N-glycans: from plant to man. Biochim. Biophys. Acta, 1473, 216-236).

Fucosylated glycans are synthesized by fucosyltransferases. Thirteen fucosyltransferase genes have thus far been identified in the human genome. FUT1 and FUT2 are α(1,2)-fucosyltransferases responsible for synthesis of the H blood group antigen and related structures (Kelly et al., 1995, Sequence and expression of a candidate for the human Secretor blood group a (1,2)fucosyltransferase gene (FUT2). Homozygosity for an enzyme-inactivating nonsense mutation commonly correlates with the non-secretor phenotype. J. Biol. Chem, 270, 4640-4649; Larsen et al., 1990, Molecular cloning, sequence, and expression of a human GDP-L-fucose:beta-D-galactoside 2-alpha-L-fucosyltransferase cDNA that can form the H blood group antigen. Proc. Natl Acad. Sci. USA, 87, 6674-6678). FUT3-FUT7 and FUT9 encode fucosyltransferases that synthesize α(1,3)- and, in the case of FUT3, α(1,4)-fucosylated glycans, such as the Lewisx and sialyl Lewisx antigens (Kaneko et al., 1999, Alpha1,3-fucosyltransferase IX (Fuc-TIX) is very highly conserved between human and mouse; molecular cloning, characterization and tissue distribution of human Fuc-TIX. FEBS Lett., 452, 237-242; Natsuka and Lowe, 1994, Enzymes involved in mammalian oligosaccharide biosynthesis. Curr. Opin. Struct. Biol, 4, 683-691). FUT8 is an α(1,6)-fucosyltransferase that directs addition of fucose to asparagine-linked GlcNAc moieties, a common feature of N-linked glycan core structures (Miyoshi et al., 1999, The α1-6-fucosyltransferase gene and its biological significance. Biochim. Biophys. Acta, 1473, 9-20.). POFUT1 encodes an O-fucosyltransferase that adds fucose directly to polypeptide chains. Finally, though not yet validated by functional studies, two additional putative α(1,3)-fucosyltransferase genes, FUT10 and FUT11, and one additional putative O-fucosyltransferase gene, O-FUT2, have been identified in the human genome by comparison with fucosyltransferase sequences in the Drosophila melanogaster genome (Roos et al., 2002, Composition of Drosophila melanogaster proteome involved in fucosylated glycan metabolism. J. Biol. Chem., 277, 3168-3175).

All fucosyltransferases utilize a nucleotide-activated form of fucose, GDP-fucose, as a fucose donor in the construction of fucosylated oligosaccharides. Two pathways have been described for synthesis of GDP-fucose in the cytosol of essentially all mammalian cells. These are termed the de novo pathway and the salvage pathway (Tonetti et al., 1998, The metabolism of 6-deoxyhexoses in bacterial and animal cells. Biochimie, 80, 923-931). The de novo pathway transforms GDP-mannose to GDP-fucose via three enzymatic reactions carried out by two proteins, GDP-mannose 4,6-dehydratase (GMD) and a second enzyme, GDP-keto-6-deoxymannose 3,5-epimerase, 4-reductase, also known as the FX protein (Tonetti et al., 1996, Synthesis of GDP-L-fucose by the human FX protein. J. Biol. Chem., 271, 27274-27279). The salvage pathway synthesizes GDP-fucose from free fucose derived from extracellular or lysosomal sources. Quantitative studies of fucose metabolism in HeLa cells indicate that greater than 90% of GDP-fucose is derived from the de novo pathway, even in cells fed radiolabeled fucose (Yurchenco and Atkinson, 1975, Fucosyl-glycoprotein and precursor polls in HeLa cells. Biochemistry, 14, 3107-3114; Yurchenco and Atkinson, 1977, Equilibration of fucosyl glycoprotein pools in HeLa cells. Biochemistry, 16, 944-953).

In mammalian cells, GDP-fucose is synthesized by two distinct pathways. The de novo pathway is characterized by conversion of GDP-mannose to GDP-4-keto-6-deoxymannose by GMD. This keto intermediate is then converted to GDP-fucose by an epimerase/reductase GDP-L-fucose synthase, also known as the FX protein (or FX enzyme). An alternative salvage pathway can yield GDP-fucose derived directly from fucose. The salvage pathway utilizes fucose that is transported into the cytosol from an extracellular origin or fucose that is liberated from catabolism of fucosylated glycans in the lysosome and then transported into the cytosol. The salvage pathway is enabled by fucose kinase and GDP-fucose pyrophosphorylase, with fucose-1-phosphate is the metabolic intermediate. GDP-fucose synthesized by these pathways is then transported into the lumen of the Golgi apparatus where it becomes available to the catalytic domains of fucosyltransferases that also localize to this membrane-delimited compartment within the secretory pathway.

### (i) Genetically Engineered Cells for Producing Afucosylated Glycoproteins

Disclosed herein are genetically engineered host animal cells such as mammalian cells capable of producing glycoproteins (e.g., exogenous glycoproteins such as antibodies) having modified glycosylation patterns (e.g., modified N-glycosylation patterns such as defucosylated N-glycans). In some embodiments, the host cells are genetically engineered to be deficient in fucose synthesis pathway. In some embodiments, the host cells are genetically engineered to be deficient in de novo fucose synthesis pathway. In some embodiments, the host cells are genetically engineered to be deficient in GDP-L-fucose synthase. In some embodiments, the host cells are genetically engineered to be deficient in FUT8. In some embodiments, the host cell comprises a double allele knock out of chromosomal TSTA3 gene, which encodes GDP-L-fucose synthase. Optionally, the host cell is also engineered to express an exogenous glycoprotein such as an antibody (e.g., anti-Tn antibody described herein). A host cell that is a knockout of a protein refers to a genetically engineered host cell that expresses the protein in a level almost undetectable compared to than that of the protein in the wild-type counterpart of the host cell, i.e., the same type of cell that does not contain the same genetic modification as the genetically engineered host cell.

A knock-out modification refers to any type of genetic modifications to a host cell (e.g., genetically engineered host cell as described herein) that results in reduced production of a target endogenous protein (e.g., GDP-L-fucose synthase) as compared to a wild-type host cell of the same type absent such genetic modification (i.e., wild-type counterpart). Reduced or elevated production of a protein of interest as described herein can be determined by comparing the production levels of the protein of interest by a population of cells of interest with that of a population of control cells (e.g., a population of genetically engineered cells as described herein versus a population of the wild-type counterpart), which are measured using the same number of cells under the same conditions (e.g., by the same assay using the same experimental conditions). In some embodiments, the host cells thus modified may produce lower levels of the target protein (e.g., GDP-L-fucose synthase), as compared to the wild-type counterpart. In some embodiments, the level of the target protein (e.g., GDP-L-fucose synthase) produced by the modified cells may not be detectable via a conventional assay. Target protein (e.g., GDP-L-fucose synthase) levels may be measured using a conventional method known in the art, such at western blot, ELISA, enzymatic activity assays, etc.

A knock-out modification may include genetic editing of an endogenous allele (e.g., TSTA3 gene) of a target protein, including, but not limited to, an insertion, deletion, or replacement within a coding region of the endogenous allele or a non-coding regulatory region of the endogenous allele, to disrupt expression of the target cytokine. As used herein, an endogenous allele is a gene allele that is naturally found within a cell (i.e., native to a cell).

Alternatively, a knock-out modification may include introducing an exogenous nucleic acid (e.g., an antisense oligonucleotide such as an interfering RNA) that suppresses expression of a target inflammatory protein as described herein. An exogenous nucleic acid refers to a nucleic acid that is not produced by the host cell before modification and is delivered into the host cell via a transfection approach, e.g., those described herein.

Any methods known in the art for down-regulating the expression of an endogenous gene in a host cell can be used to reduce the production level of a target endogenous cytokine/protein as described herein.

In some instances, a gene editing method can be performed to modify an endogenous allele of a gene of the target protein (e.g., in a coding region or a non-coding regulatory region) so as to reduce expression of the target endogenous protein (e.g., GDP-L-fucose synthase). A gene editing method may involve use of an endonuclease that is capable of cleaving the target region in the endogenous allele. Non-homologous end joining in the absence of a template nucleic acid may repair double-strand breaks in the genome and introduce mutations (e.g., insertions, deletions and/or frameshifts) into a target site. Gene editing methods are generally classified based on the type of endonuclease that is involved in generating double stranded breaks in the target nucleic acid. Examples include, but are not limited to, Clustered Regularly Interspaced Short Palindromic Repeats (CRISPR)/endonuclease systems, transcription activator-like effector-based nuclease (TALEN), zinc finger nucleases (ZFN), endonucleases (e.g., ARC homing endonucleases), meganucleases (e.g., mega-TALs), or a combination thereof.

Cleavage of a gene region may comprise cleaving one or two strands at the location of the target allele by an endonuclease. In some embodiments, the cleavage event may be followed by repairing the cleaved target polynucleotide by homologous recombination with an exogenous template polynucleotide, leading to insertion, deletion, or substitution of one or more nucleotides of the target nucleotide sequence. Such gene editing can result in decreased transcription of a target gene

In some embodiments, genetic modification of immune cells as described herein is performed using the Clustered Regularly Interspaced Short Palindromic Repeats (CRISPR)/endonuclease technology known in the art. The CRISPR/endonuclease systems have been adapted for use in both prokaryotic and eukaryotic cells. Gene editing with CRISPR generally relies on expression of at least two components: a guide RNA sequence that recognizes a target nucleic acid sequence and an endonuclease (e.g., including Cpf1 and Cas9). As the name suggests, the A guide RNA helps direct the endonuclease to a target site, which typically contains a nucleotide sequence that is complementary (partially or completely) to the gRNA or a portion thereof. In some instance, the guide RNA is a two-piece guide RNA complex comprises a protospacer sequence fragment that is complementary to the target nuclei acid sequence and a third component, a scaffold RNA fragment. In some instances, the scaffold RNA, is required to aid in recruiting the endonuclease to the target site. In some instances, the guide RNA is a single guide RNA (sgRNA) that combines comprises both the protospacer sequence and the scaffold RNA sequence. In some embodiments, the sgRNA is designed to target a protein in the fucose synthesis pathway. In some embodiments, the sgRNA is designed to target TSTA3 gene. In some embodiments, the sgRNA is designed to target exon 4 of the TSTA3 gene. An exemplary protospacer sequence of the sgRNA can be: TCACCATGGTCTCATCTATC (SEQ ID NO: 17). Once at the target site, the endonuclease can generate a double strand break.

The target nucleic acid for use with the CRISPR system is flanked on the 3′ side by a protospacer adjacent motif (PAM) that may interact with the endonuclease and be further involved in targeting the endonuclease activity to the target nucleic acid. It is generally thought that the PAM sequence flanking the target nucleic acid depends on the endonuclease and the source from which the endonuclease is derived. For example, for Cas9 endonucleases that are derived from Streptococcus pyogenes, the PAM sequence is NGG. For Cas9 endonucleases derived from Staphylococcus aureus, the PAM sequence is NNGRRT. For Cas9 endonucleases that are derived from Neisseria meningitidis, the PAM sequence is NNNNGATT. For Cas9 endonucleases derived from Streptococcus thermophilus, the PAM sequence is NNAGAA. For Cas9 endonuclease derived from Treponema denticola, the PAM sequence is NAAAAC. For a Cpf1 nuclease, the PAM sequence is TTN.

A CRISPR/endonuclease system that hybridizes with a target sequence in the locus of an endogenous cytokine may be used to knock out the cytokine of interest. A gRNA hybridizes to (complementary to, partially or completely) a target nucleic acid sequence (e.g., the endogenous locus of TSTA3 gene) in the genome of a host cell. The gRNA or portion thereof that hybridizes to the target nucleic acid may be between 15-25 nucleotides, 18-22 nucleotides, or 19-21 nucleotides in length. In some instances, the gRNA sequence that hybridizes to the target nucleic acid is 15, 16, 17, 18, 19, 20, 21, 22, 23, 24, or 25 nucleotides in length. In some examples, the gRNA sequence that hybridizes to the target nucleic acid is between 10-30, or between 15-25, nucleotides in length.

In some examples, the gRNA sequence is at least 50%, 55%, 60%, 65%, 70%, 75%, 80%, 85%, 90%, 95%, 96%, 97%, 98%, 99%, or at least 100% complementary to a target nucleic acid (see also U.S. Pat. No. 8,697,359, which is incorporated by reference for its teaching of complementarity of a gRNA sequence with a target polynucleotide sequence). It has been demonstrated that mismatches between a CRISPR guide sequence and the target nucleic acid near the 3′ end of the target nucleic acid may abolish nuclease cleavage activity (see, e.g., Upadhyay, et al. Genes Genome Genetics (2013) 3(12):2233-2238). In some examples, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The “percent identity” of two nucleic acids is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

A variety of CRISPR/endonuclease systems are known in the art and modifications are regularly and numerous references describe rules and parameters that are used to guide the design of CRISPR/endonuclease systems (e.g., including Cas9 target selection tools). See, e.g., Hsu et al., Cell, 157(6):1262-78, 2014.

In some instances, genetic modification of the immune cells as described herein is performed using the TALEN technology known in the art. TALENs are engineered restriction enzymes that can specifically bind and cleave a desired target DNA molecule. A TALEN typically contains a Transcriptional Activator-Like Effector (TALE) DNA-binding domain fused to a DNA cleavage domain. The DNA binding domain may contain a highly conserved 33-34 amino acid sequence with a divergent 2 amino acid RVD (repeat variable dipeptide motif) at positions 12 and 13. The RVD motif determines binding specificity to a nucleic acid sequence and can be engineered according to methods known to those of skill in the art to specifically bind a desired DNA sequence (see, e.g., Juillerat et al., Scientific reports, 5:8150, 2015; Miller et. al., Nature Biotechnology 29 (2): 143-8, 2011; Zhang et al., Nature Biotechnology 29 (2): 149-53, 2011; Geiβler et al., PLoS ONE 6 (5): e19509, 2011; Boch, Nature Biotechnology 29 (2): 135-6, 2011; Boch, et. al., Science 326 (5959): 1509-12, 2009; and Moscou et al., Science 326 (5959): 1501, 2009. The DNA cleavage domain may be derived from the FokI endonuclease, which is active in many different cell types. The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. See, e.g., Miller et al., Nature Biotech. 29: 143-8, 2011.

A TALEN specific to a target gene of interest can be used inside a cell to produce a double-stranded break (DSB). A mutation can be introduced at the break site if the repair mechanisms improperly repair the break via non-homologous end joining. For example, improper repair may introduce a frame shift mutation.

In some examples, zinc finger nucleases (ZFNs), which are known in the art, may be used to generate a population of modified immune cells described herein. Zinc finger nucleases (ZFNs) are restriction enzymes comprised of an engineered zinc finger DNA binding domain linked to the catalytic domain of the type II endonuclease FokI. The zinc finger DNA binding domain of each ZFN targets the linked FokI endonuclease to a specific site in the genome. Since FokI functions only as a dimer, a pair of ZFNs is typically engineered to bind to cognate target “half-site” sequences on opposite DNA strands. The target “half-site” sequences are generally spaced such the catalytically active FokI dimer may form between them. Upon dimerization of the FokI domain, a DNA double-strand break is generated between the ZFN half-sites. As mentioned above, non-homologous end joining may introduce mutations, while homology-directed repair may be used to introduce an exogenous nucleic acid.

Many gene editing systems using ZFNs and considerations for design of ZFNs have been described; see, e.g., Segal et al., Proc Natl Acad Sci USA 96(6):2758-63, 1999; Dreier B et al., J Mol Biol. 303(4):489-502, 2000; Liu Q et al., J Biol Chem. 277(6):3850-6, 2002; Dreier et al., J Biol Chem 280(42):35588-97, 2005; and Dreier et al., J Biol Chem. 276(31):29466-78, 2001.

Meganucleases (or homing endonucleases), which are sequence-specific endonucleases that recognize long DNA targets (often between 14 and 40 base pairs) may also be introduced using any method known in the art to genetically engineer any of the modified cells described herein. There are at least six families of meganucleases and they are often classified based on structural motifs, including LAGLIDADG, GIY-YIG, HNH, His-Cys box, PD-(D/E)XK and Vsr-like. Non limiting examples of meganucleases include PI-SceI, I-CreI and I-TevI.

Various gene editing systems using meganucleases, including modified meganucleases, have been described in the art; see, e.g., the reviews by Steentoft et al., Glycobiology 24(8):663-80, 2014; Belfort and Bonocora, Methods Mol Biol. 1123:1-26, 2014; Hafez and Hausner, Genome 55(8):553-69, 2012; and references cited therein.

Hybrid nucleases including MegaTAL may also be used. MegaTALs are a fusion of a TALE DNA binding domain with a catalytically active meganuclease. Such nucleases harness the DNA binding specificity of TALEs and the sequence cleavage specificity of meganucleases. See, e.g., Boissel et al., NAR, 42: 2591-2601, 2014.

In some embodiments, the genetically engineered host cells as described herein are capable of producing glycoproteins (e.g., anti-Tn antibody) having modified glycosylation as compared with the wild-type counterpart. In some embodiments, the genetically engineered host cells as described herein are capable of producing glycoproteins (e.g., anti-Tn antibody) having modified fucosylation as compared with the wild-type counterpart. In some embodiments, the genetically engineered host cells are deficient in de novo fucose synthesis pathway. In some embodiments, the genetically engineered host cells are capable of fucosylation of glycoproteins using the salvage pathway. In some embodiments, the fucosylation activity of the genetically engineered host cells as described herein are dependent on the free L-fucose level in the culture media. In some embodiments, the genetically engineered host cells are capable of producing afucosylated glycoprotein (e.g., anti-Tn antibody) in the absence of L-fucose in the culture media. In some embodiments, the genetically engineered host cells are capable of producing afucosylated glycoprotein (e.g., anti-Tn antibody) in the presence of L-fucose in the culture media. In some embodiments, L-fucose is supplied to the culture media exogenously. In some embodiments, the genetically engineered host cells described herein are capable of producing at least 50%, at least 60%, at least 70%, at least 80%, at least 90%, at least 95%, at least 98%, at least 99%, or 100% afucosylated glycoproteins (e.g., anti-Tn antibody).

Non-limiting examples of suitable host cell to be genetically modified as described herein are HEK293 cells, Chinese hamster ovary (CHO) cells, dhFr− CHO cell, HeLa cells, HT-1080 cells, PER.C6, HKB-11 cells, CAP cells, HuH07 cells, NS0 cells, HKB11, Sp2/0 cells, BHK cells, and C127 cells, or derivatives or clones of such cells, and other eukaryotic cells engineered to depend on exogenous fucose for generating fucosylated antibodies. It is also within the scope of disclosure that any cells having endogenous TSTA3 locus can be genetically modified to obtain the host cells described herein. In some embodiments, the host cell that is genetically modified are

### (ii) Afucosylated Anti-Tn Antibodies

The genetically engineered host cells described herein can be used for producing afucosylated antibodies (e.g., anti-Tn antibodies).

As used herein, “the percentage of fucosylated antibodies” refers to the percentage of the antibody in the entire antibody population that has fucose residues. As used herein, “afucocylated antibodies” refers to antibodies that do not contain any fucose residue.

As used herein, “the amount of fucose” means the amount of fucose residues within the N-glycan at Asn297, related to the sum of all glycostructures attached to Asn297 (e.g. complex, hybrid and high mannose structures) measured by MALDI-TOF mass spectrometry and calculated as average value. The relative amount of fucose is the percentage of fucose-containing structures related to all glycostructures identified in an N-Glycosidase F treated sample (e.g. complex, hybride and oligo- and high-mannose structures, resp.) by MALDI-TOF or equivalent mass spectrometry technologies.

In some embodiments, the anti-Tn antibody can be produced by the genetically engineered host cell described herein (e.g., host cells that are KO for GDP-L-fucose synthase). In some embodiments, the anti-Tn antibody described herein comprises a Fc portion that lacks any fucose residues. In some embodiments, the afucosylated antibody described herein (e.g., anti-Tn antibody) has an increased level of antibody-dependent cellular cytotoxicity (ADCC) activity when compared to antibodies produced by WT counterpart cells. In some embodiments, the afucosylated antibody described herein (e.g., anti-Tn antibody) has an increased level of antibody-dependent cellular cytotoxicity (ADCC) activity when compared to fucosylated antibodies.

In one aspect, the afucosylated antibodies disclosed herein (e.g., afucosylated anti-Tn antibodies) have a high ADCC activity. Antibodies can act as a therapeutic through various mechanisms, one of which is through ADCC activity. “Antibody-dependent cell-mediated cytotoxicity” or “ADCC”, as used herein, refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIIL FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or Presta U.S. Pat. No. 6,737,056 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998). Therapeutic antibodies that bind to cellular receptors on a target cell, and that include the Fc glycosylation site can also bind the Fc-receptor resulting in the anchoring of cells expressing the Fc-receptor to the target cell. The affinity of binding of the Fc regions of antibodies generally is dependent on the nature of the glycosylation of the Fc glycosylation site. The Fc receptor is found on a number of immune cells including natural killer cells, macrophages, neutrophils, and mast cells. Binding to the Fc receptor results in the immune cells inducing cytokines (such as IL-2) and phagocytosis to kill the target cell. In some embodiments, a population of antibodies that has an increased level of antibody-dependent cellular cytotoxicity (ADCC) activity is a population of antibodies that shows increased binding to cells expressing CD16 as compared to a population of antibodies that does not have an increased level of antibody-dependent cellular cytotoxicity (ADCC) activity. In some embodiments a population of antibodies that has an increased level of antibody-dependent cellular cytotoxicity (ADCC) activity is a population of antibodies that shows increased induction of IL-2 production (e.g., in natural killer cells) as compared to a population of antibodies that has does not have an increased level of antibody-dependent cellular cytotoxicity (ADCC) activity. Commercial kits for determining ADCC activity can be purchased for instance from Genscript (Piscataway, NJ) and Promega (Madison, WI). In some embodiments, determining ADCC activity is performed by evaluating the ability to bind CD16.

In some embodiments, the afucosylated antibody (e.g., afucosylated anti-Tn antibody) has an increased level of antibody-dependent cellular cytotoxicity (ADCC) when compared to a population of fucosylated antibodies. In some embodiments, the ADCC activity of afucosylated antibodies is at least 10% higher, at least 20% higher, at least 30% higher, at least 40% higher, at least 50% higher, at least 60% higher, at least 70% higher, at least 80% higher, at least 90% higher, at least 100% higher, at least 200% higher, at least 300% higher, at least 400% higher, at least 500% higher, at least 600% higher, at least 700% higher, at least 800% higher, at least 900% higher, or more when compared to fucosylated antibodies. In some embodiments, the ADCC activity of afucosylated antibodies is at least 1.1 times higher, 1.2 times higher, 1.3 times higher, 1.4 times higher, 1.5 times higher, 1.6 times higher, 1.7 times higher, 1.8 times higher, 1.9 times higher, 2 times higher, 3 times higher, 5 times higher, 10 times higher, 100 times higher or more when compared to fucosylated antibodies.

(iii) Producing Glycoproteins Having Modified Fucosylation Level

Also, with the scope of the present disclosure, is the production of any glycoprotein using the genetically engineered host cells (e.g., cells that are KO for GDP-L-fucose synthase).

In some examples, the glycoprotein of interest is an antibody. Exemplary antibodies include, but are not limited to, abciximab (glycoprotein IIb/IIIa; cardiovascular disease), adalimumab (TNF-α, various auto-immune disorders, e.g., rheumatoid arthritis), alemtuzumab (CD52; chronic lymphocytic leukemia), basiliximab (IL-2Rα receptor (CD25); transplant rejection), bevacizumab (vascular endothelial growth factor A; various cancers, e.g., colorectal cancer, non-small cell lung cancer, glioblastoma, kidney cancer; wet age-related macular degeneration), catumaxomab, cetuximab (EGF receptor, various cancers, e.g., colorectal cancer, head and neck cancer), certolizumab (e.g., certolizumab pegol) (TNF alpha; Crohn's disease, rheumatoid arthritis), Daclizumab (IL-2Rα receptor (CD25); transplant rejection), eculizumab (complement protein C5; paroxysmal nocturnal hemoglobinuria), efalizumab (CD11a; psoriasis), gemtuzumab (CD33; acute myelogenous leukemia (e.g., with calicheamicin)), ibritumomab tiuxetan (CD20; Non-Hodgkin lymphoma (e.g., with yttrium-90 or indium-111)), infliximab (TNF alpha; various autoimmune disorders, e.g., rheumatoid arthritis) Muromonab-CD3 (T Cell CD3 receptor; transplant rejection), natalizumab (alpha-4 (α4) integrin; multiple sclerosis, Crohn's disease), omalizumab (IgE; allergy-related asthma), palivizumab (epitope of RSV F protein; Respiratory Syncytial Virus infection), panitumumab (EGF receptor; cancer, e.g., colorectal cancer), ranibizumab (vascular endothelial growth factor A; wet age-related macular degeneration), rituximab (CD20; Non-Hodgkin lymphoma), tositumomab m (CD20; Non-Hodgkin lymphoma), trastuzumab (ErbB2; breast cancer).

In some examples, the glycoprotein of interest is a cytokine. Examples include, but are not limited to, interferons (e.g., IFN-α, INF-β, or INF-γ), interleukins (e.g., IL-2, IL-3, IL-4, IL-5, IL-6, IL-7, IL-12), and colony stimulating factors (e.g., G-CSF, GM-CSF, M-CSF). The IFN can be, e.g., interferon alpha 2a or interferon alpha 2b. See, e.g., Mott H R and Campbell I D. “Four-helix bundle growth factors and their receptors: protein-protein interactions.” Curr Opin Struct Biol. 1995 February; 5(1):114-21; Chaiken I M, Williams W V. “Identifying structure-function relationships in four-helix bundle cytokines: towards de novo mimetics design.” Trends Biotechnol. 1996 October; 14(10):369-75; Klaus W, et al., “The three-dimensional high resolution structure of human interferon alpha-2a determined by heteronuclear NMR spectroscopy in solution”. J. Mol Biol., 274(4):661-75, 1997, for further discussion of certain of these cytokines.

In addition, the protein of interest may be a protein that is approved by the US Food & Drug Administration (or an equivalent regulatory authority such as the European Medicines Evaluation Agency) for use in treating a disease or disorder in humans. Such proteins may or may not be one for which a PEGylated version has been tested in clinical trials and/or has been approved for marketing. In some instances, the protein of interest is an Fc-fusion protein, including, but not limited to, abatacept, entanercept, IL-2-Fc fusion protein, CD80-Fc fusion protein, and PDL1-Fc fusion protein.

Further, the protein of interest may be a neurotrophic factor, i.e., a factor that promotes survival, development and/or function of neural lineage cells (which term as used herein includes neural progenitor cells, neurons, and glial cells, e.g., astrocytes, oligodendrocytes, microglia). For example, in some embodiments, the target protein is a factor that promotes neurite outgrowth. In some embodiments, the protein is ciliary neurotrophic factor (CNTF; a four-helix bundle protein) or an analog thereof such as Axokine, which is a modified version of human Ciliary neurotrophic factor with a 15 amino acid truncation of the C terminus and two amino acid substitutions, which is three to five times more potent than CNTF in in vitro and in vivo assays and has improved stability properties.

Alternatively, the protein of interest can be an enzyme, e.g., an enzyme that is important in metabolism or other physiological processes. As is known in the art, deficiencies of enzymes or other proteins can lead to a variety of disease. Such diseases include diseases associated with defects in carbohydrate metabolism, amino acid metabolism, organic acid metabolism, porphyrin metabolism, purine or pyrimidine metabolism, lysosomal storage disorders, blood clotting, etc. Examples include Fabry disease, Gaucher disease, Pompe disease, adenosine deaminase deficiency, asparaginase deficiency, porphyria, hemophilia, and hereditary angioedema. In some embodiments, a protein is a clotting or coagulation factor (e.g., factor VII, VIIa, VIII or IX). In other embodiments a protein is an enzyme that plays a role in carbohydrate metabolism, amino acid metabolism, organic acid metabolism, porphyrin metabolism, purine or pyrimidine metabolism, and/or lysosomal storage, wherein exogenous administration of the enzyme at least in part alleviates the disease.

Further, the protein of interest can be a hormone, such as insulin, growth hormone, Luteinizing hormone, follicle-stimulating hormone, and thyroid-stimulating hormone. The protein of interest can also be a growth factor, including, but not limited to, adrenomedullin (AM), angiopoietin (Ang), autocrine motility factor, bone morphogenetic proteins (BMPs), brain-derived neurotrophic factor (BDNF), epidermal growth factor (EGF), erythropoietin (EPO) fibroblast growth factor (FGF), glial cell line-derived neurotrophic factor (GDNF), granulocyte colony-stimulating factor (G-CSF), granulocyte macrophage colony-stimulating factor (GM-CSF), growth differentiation factor-9 (GDF9), healing factor, hepatocyte growth factor (HGF) hepatoma-derived growth factor (HDGF), insulin-like growth factor (IGF), keratinocyte growth factor (KGF), migration-stimulating factor (MSF), myostatin (GDF-8), nerve growth factor (NGF) and other neurotrophins, platelet-derived growth factor (PDGF), thrombopoietin (TPO), transforming growth factor alpha (TGF-α), transforming growth factor beta (TGF-β), tumor necrosis factor-alpha (TNF-α), vascular endothelial growth factor (VEGF), and placental growth factor (PGF).

### IV. Pharmaceutical Composition

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical composition disclosed herein (e.g., compositions containing the anti-Tn antibody, the encoding nucleic acids, vectors or host cells) may further comprise a suitable buffer agent. A buffer agent is a weak acid or base used to maintain the pH of a solution near a chosen value after the addition of another acid or base. In some examples, the buffer agent disclosed herein can be a buffer agent capable of maintaining physiological pH despite changes in carbon dioxide concentration (produced by cellular respiration). Exemplary buffer agents include, but are not limited to a HEPES (4-(2-hydroxyethyl)-1-piperazineethanesulfonic acid) buffer, Dulbecco's phosphate-buffered saline (DPBS) buffer, or Phosphate-buffered Saline (PBS) buffer. Such buffers may comprise disodium hydrogen phosphate and sodium chloride, or potassium dihydrogen phosphate and potassium chloride.

In some embodiments, the buffer agent in the pharmaceutical composition described herein may maintain a pH value of about 5-8. For example, the pH of the pharmaceutical composition can be about 7.1, 7.2, 7.3, 7.4, 7.5, 7.6, 7.7, 7.8, 7.9, or 8.0. In other examples, the pharmaceutical composition may have a pH value lower than 7, for example, about 7, 6.8, 6.5, 6.3, 6, 5.8, 5.5, 5.3, or 5.

The pharmaceutical composition disclosed herein (e.g., compositions containing the anti-Tn antibody, the encoding nucleic acids, vectors or host cells) comprises one or more suitable salts. A salt is an ionic compound that can be formed by the neutralization reaction of an acid and a base. (Skoog, D. A; West, D. M.; Holler, J. F.; Crouch, S. R. (2004). “chapters 14-16”. Fundamentals of Analytical Chemistry (8th ed.)). Salts are composed of related numbers of cations (positively charged ions) and anions (negative ions) so that the product is electrically neutral (without a net charge). An ion, as described herein, are atoms or molecules which have gained or lost one or more valence electrons giving the ion a net positive or negative charge. If the chemical species has more protons than electrons, it carries a net positive charge. If there are more electrons than protons, the species has a negative charge.

A cation (+), as described herein, is an ion with fewer electrons than protons, giving it a positive charge. (Douglas W. Haywick, (2007-2008). “Elemental Chemistry”). A cation with one positive charge can be called a monovalent cation; a cation with more than one positive charge can be called a polyvalent or multivalent cation. Non limiting examples of monovalent cations are hydrogen (H⁺), sodium (Na⁺), potassium (K⁺), ammonium (NH⁴⁺) Lithium (Li⁺), cuprous (Cu⁺), silver (Ag⁺), etc. Non limiting examples of multivalent cations are magnesium (Mg²⁺), calcium (Ca²⁺), barium (Ba²⁺), beryllium (Be²⁺), cupric (Cu²⁺), ferrous (Fe²⁺), ferric (Fe³⁺), lead(II) (Pb²⁺), lead(IV) (Pb⁴⁺), manganese(II) (Mn²⁺), strontium (Sr2⁺), tin(IV) (Sn⁴⁺), zinc (Zn²⁺), etc.

An anion, as described herein, is an ion with more electrons than protons, giving it a net negative charge. Non limiting examples of anions are azide (N₃⁻), bromide (Br⁻), chloride (Cl⁻), fluoride (F⁻), hydride (H⁻), iodide (I⁻), nitride (N⁻), Oxide (O²⁻), sulfide (S₂⁻), carbonate (CO₃²⁻), hydrogen carbonate (HCO₃⁻), hydrogen sulfate (HSO₄⁻), hydroxide (OH⁻), dihydrogen phosphate (H₂PO₄⁻), sulfate (SO₄²⁻), sulfite (SO₃²⁻), silicate (SiO₃²⁻), etc.

Suitable salts for use in the pharmaceutical compositions described herein may contain a monovalent cation and a monovalent or multi-valent anion. Alternatively, the salts for use in the pharmaceutical compositions described herein may contain a monovalent or multi-valent cation and a monovalent anion. Exemplary salts include, but are not limited to, potassium chloride (KCl), sodium chloride (NaCl), calcium chloride (CaCl₂)), Magnesium chloride (MgCl₂), Magnesium Sulfate (MgSO₄), Sodium Bicarbonate (NaHCO₃), Ammonium sulfate ((NH₄)₂SO₄), calcium carbonate (Ca₂CO₃), or a combination thereof.

The pharmaceutical composition disclosed herein (e.g., compositions containing the anti-Tn antibody, the encoding nucleic acids, vectors or host cells) comprises one or more suitable surface-active agents, such as a surfactant. Surfactants are compounds that lower the surface tension (or interfacial tension) between two liquids, between a gas and a liquid, or between a liquid and a solid. Surfactants may act as detergents, wetting agents, emulsifiers, foaming agents, and dispersants. Suitable surfactants include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

The pharmaceutical composition disclosed herein (e.g., compositions containing the anti-Tn antibody, the encoding nucleic acids, vectors or host cells) may also comprise one or more antioxidants. An antioxidant, as used herein, is an agent that prevents or delays oxidative degradation of the active ingredients contained in the composition. The antioxidants used herein may be phenolic antioxidants (sometimes called true antioxidants), reducing agents, or chelating agents. Phenolic antioxidants are sterically hindered phenols that react with free radicals, blocking the chain reaction. Reducing agents are compounds that have lower redox potentials and, thus, are more readily oxidized than the drug they are intended to protect. Reducing agents scavenge oxygen from the medium and, thus, delay or prevent drug oxidation. Chelating agents are sometimes called antioxidant synergists. Metal ions, such as Co²⁺, Cu²⁺, Fe³⁺, Fe²⁺, and Mn²⁺, shorten the induction period and increase the oxidation rate. Trace amounts of these metal ions are frequently introduced to drug products during manufacturing. Chelating agents do not possess antioxidant activity as such, but enhance the action of phenolic antioxidants by reacting with catalyzing metal ions to make them inactive.

The pharmaceutical composition disclosed herein (e.g., compositions containing the anti-Tn antibody, the encoding nucleic acids, vectors or host cells) may also comprise a sugar derivative. A sugar derivative, as used herein, encompasses sugars and organic compounds derived from sugar. In some examples, the sugar derivative can be a non-reducing sugar, a sugar alcohol, a polyol, a disaccharide or a polysaccharide.

The pharmaceutical composition disclosed herein (e.g., compositions containing the anti-Tn antibody, the encoding nucleic acids, vectors or host cells) to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and mcresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition disclosed herein (e.g., compositions containing the anti-Tn antibody, the encoding nucleic acids, vectors or host cells) comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules,

respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition disclosed herein (e.g., compositions containing the anti-Tn antibody, the encoding nucleic acids, vectors or host cells) can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L20 glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In other examples, the pharmaceutical composition disclosed herein (e.g., compositions containing the anti-Tn antibody, the encoding nucleic acids, vectors or host cells) can be formulated in a sustained release format, which affects binding selectively to tissue or tumors by implementing certain protease biology technology, for example, by peptide masking of the antibody's antigen binding site to allow selective protease cleavability by one or multiple proteases in the tumor microenvironment, such as Probody™ or Conditionally Active Biologics™. An activation may be formulated to be reversible in a normal microenvironment.

The pharmaceutical composition disclosed herein (e.g., compositions containing the anti-Tn antibody, the encoding nucleic acids, vectors or host cells) to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical composition disclosed herein (e.g., compositions containing the anti-Tn antibody, the encoding nucleic acids, vectors or host cells) can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an 10 oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 .im, particularly 0.1 and 0.5 .im, and have a pH in the range of 5.5 to 8.0. The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### V. Therapeutic Applications

Any of the antibodies, as well as the encoding nucleic acids or nucleic acid sets, vectors comprising such, or host cells comprising the vectors, described herein are useful for treating disorders associated with Tn antigen (e.g., cancer). Disorders associated with Tn antigen, as used herein, refer to any medical condition associated with increased levels of Tn antigen.

To practice the method disclosed herein, an effective amount of the pharmaceutical composition disclosed herein (e.g., compositions containing the anti-Tn antibody, the encoding nucleic acids, vectors or host cells) can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized, and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having inflammatory diseases, autoimmune diseases, cancer, infectious diseases or other disorders requiring modulation of the immune response. A subject having a target disease or disorder can be identified by routine medical examination, e.g., laboratory tests, organ functional tests, CT scans, or ultrasounds. A subject suspected of having any of such target disease/disorder might show one or more symptoms of the disease/disorder. A subject at risk for the disease/disorder can be a subject having one or more of the risk factors for that disease/disorder.

The methods and compositions described herein may be used to treat cancer. Examples of autoimmune diseases are leukemia, gastric carcinoma, adenocarcinoma, mesothelioma, breast cancer, pancreatic ductal adenocarcinoma, colitis-associated colorectal cancer (CAC), or hypereosinophilic syndrome (HES), colorectal cancer, prostate cancer, lung cancer, ovarian cancer, stomach cancer, bladder cancer, cervix cancer, endometrial cancer, glioblastomas, salivary gland cancer, nasopharyngeal cancers, skin cancers (e.g., basal cell carcinomas, squamous cell carcinomas), renal cell carcinomas, ductal carcinomas, invasive ductal carcinomas, adenocarcinomas, esophageal cancer, unspecified gastrointestinal cancer, pancreatic cancer, and melanoma, as well as sarcomas, including angiosarcoma, bone sarcoma, osteosarcoma, neurofibrosarcomas, rhabdomyosarcoma, soft tissue sarcoma, synovial sarcoma, condrosarcoma, chordomas, Kaposi's sarcoma, giant cell tumor of the bone, leiomyosarcoma, desmoid-type fibromatosis, Ewing's sarcoma, fibroblastic sarcoma, gastrointestinal stromal tumors, as well as lymphomas, leukemias, or thymomas. It is within the scope of the present disclosure that any type of cancer cell expressing Tn antigen can be treated by the antibodies and compositions described herein. Subjects having or at risk for various cancers can be identified by routine medical procedures.

As used herein, “an effective amount” refers to the amount of each active agent required to confer therapeutic effect on the subject, either alone or in combination with one or more other active agents. In some embodiments, the therapeutic effect is reduced Tn antigen, or reduced tumor burden.

Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation.

It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the antagonist. To assess efficacy of the antagonist, an indicator of the disease/disorder can be followed.

Generally, for administration of any of the antibodies described herein, an initial candidate dosage can be about 2 mg/kg. For the purpose of the present disclosure, a typical daily, weekly, every two weeks, or every three weeks dosage might range from about any of 0.1 μg/kg to 3 μg/kg to 30 μg/kg to 100 μg/kg to 300 μg/kg to 0.6 mg/kg, 1 mg/kg, 3 mg/kg, to 10 mg/kg, to 30 mg/kg to 100 mg/kg or more, depending on the factors mentioned above. For repeated administrations over several days, weeks, months, or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 3 mg/kg every 3 weeks, followed by a maintenance dose of about 1 mg/kg of the antibody once in 6 weeks, or followed by a maintenance dose of about 1 mg/kg every 3 weeks. However, other dosage regimens may be useful, depending on the pattern of pharmacokinetic decay that the practitioner wishes to achieve. For example, dosing of 1 mg/kg once in every 3 weeks in combination treatment with at least one additional immune therapy agent is contemplated. In some embodiments, dosing ranging from about 3 μg/mg to about 3 mg/kg (such as about 3 μg/mg, about 10 μg/mg, about 30 μg/mg, about 100 μg/mg, about 300 μg/mg, about 1 mg/kg, and about 3 mg/kg) may be used. In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.1 to 5.0 mg/kg may be administered. In some examples, the dosage of the anti-Tn antibody described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the antagonist, and the discretion of the attending physician. Typically, the clinician will administer an antibody, until a dosage is reached that achieves the desired result. In some embodiments, the desired result is a reduction of the size of the tumor, increased progression free survival period and/or overall survival. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying the development or progression of the disease, or reducing disease severity.

Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

In some embodiments, the antibodies described herein are administered to a subject in need of the treatment at an amount sufficient to inhibit the activity of the target antigen by at least 20% (e.g., 30%, 40%, 50%, 60%, 70%, 80%, 90% or greater) in vivo. In other embodiments, the antibody is administered in an amount effective in reducing the activity level of a target antigen by at least 20% (e.g., 30%, 40%, 50%, 60%, 70%, 80%, 90% or greater).

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered parenterally, topically, orally, by inhalation spray, rectally, nasally, buccally, vaginally or via an implanted reservoir. The term “parenteral” as used herein includes subcutaneous, intracutaneous, intravenous, intraperitoneal, intratumor, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline. Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods and Applications of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e., dose, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents. Treatment efficacy for a target disease/disorder can be assessed by methods well-known in the art.

The anti-Tn antibody and treatment methods involving such as described in the present disclosure may be utilized in combination with other types of therapy for the target disease or disorder disclosed herein. The term “in combination” in this context means that the antibody composition and the therapeutic agent are given either simultaneously or sequentially. Examples include chemotherapy, immune therapy (e.g. therapies involving anti-inflammatory drugs, immunosuppressant, therapeutic antibodies, antibodies, CAR T cells, or cancer vaccines), surgery, radiation, gene therapy, and so forth, or anti-infection therapy. Such therapies can be administered simultaneously or sequentially (in any order) with the treatment according to the present disclosure.

For example, the combination therapy can include the anti-Tn antibody and pharmaceutical composition described herein, co-formulated with and/or co-administered with, at least one additional therapeutic agent. In one embodiment, the additional agent is a cancer chemotherapeutic agent e.g. oxaliplatin, gemcitabine, docetaxel. In another embodiment, the additional agent can be disease modifying antirheumatic drugs (DMARDs) e.g. methotrexate, azathioprine, chloroquine, hydroxychloroquine, cyclosporin A, sulfasalazine, for RA treatment. Such combination therapies may advantageously utilize lower dosages of the administered therapeutic agents, thus preventing possible toxicities or complications associated with the various monotherapies.

When the antibody composition described here is co-used with a second therapeutic agent, a sub-therapeutic dosage of either the composition or of the second agent, or a sub-therapeutic dosage of both, can be used in the treatment of a subject having, or at risk of developing a disease or disorder associated with the Tn antigen. A “sub-therapeutic dose” as used herein refers to a dosage, which is less than that dosage which would produce a therapeutic result in the subject if administered in the absence of the other agent or agents. Thus, the sub-therapeutic dose of an agent is one which would not produce the desired therapeutic result in the subject in the absence of the administration of the anti-Tn antibody described herein. Therapeutic doses of many agents that are in clinical use are well known in the field of medicine, and additional therapeutic doses can be determined by those of skill without undue experimentation. Therapeutic dosages have been extensively described in references such as Remington's Pharmaceutical Sciences, 18th ed., 1990; as well as many other medical references relied upon by the medical profession as guidance for the treatment of diseases and disorders. Additional useful agents see also Physician's Desk Reference, 59.sup.th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

In some embodiments, the anti-Tn antibodies described herein, can be conjugated to with a therapeutic agent for the treatment of the intended diseases. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker.

In some embodiments, the anti-Tn antibody may be conjugated to a cytotoxic agent. Non-limiting examples of the cytotoxic agent are dolastin 10, zogamicin, monomethyl auristatin E (MMAE), cryptophycin and analogs thereof, enediyne antiobiotics including wesperamicin and maytansines (emtasine DM1/DM4), calicheamicin, capecitabine, lapatinib, anthracyclines, duocarmycins, pyyrolobenzodiazepines. In some embodiments, the anti-Tn antibody may be conjugated to a toxin. Non-limiting examples of the toxins are Pseudomonas exotoxin, or diphtheria toxin. In some embodiments, the anti-Tn-antibody is conjugated to an oligonucleotide (e.g., anti-sense oligonucleotide, siRNA, or miRNA).

The present disclosure also features chimeric antigen receptors targeting Tn antigen and immune cells expressing such. Chimeric antigen receptors (CARs) as disclosed herein are artificial cell-surface receptors that redirect binding specificity of immune cells (e.g., T cells) expressing such to Tn-antigen+ cells, thereby eliminating the target cells via, e.g., the effector activity of the immune cells. A CAR construct often comprises an extracellular antigen binding domain fused to at least an intracellular signaling domain. Cartellieri et al., J Biomed Biotechnol 2010:956304, 2010. The extracellular antigen binding domain, which can be a single-chain antibody fragment (scFv), is specific to a CTLA-4 antigen and the intracellular signaling domain can mediate a cell signaling that lead to activation of immune cells. As such, immune cells expressing a CAR construct specific to Tn antigen can bind to target cells expressing Tn antigen, leading to activation of the immune cells and elimination of the target cells.

Any of the anti-Tn antibodies described herein can be used to produce the CAR constructs also described herein. For example, the VH and VL domains of an anti-Tn antibody can be fused to the intracellular signaling domain(s) to produce a CAR construct using the conventional recombinant technology. In some examples, the VH and VL domains of an anti-Tn antibody are connected via a peptide linker to form a scFv fragment. The CAR construct disclosed herein may comprise one or more intracellular signaling domains. In some examples, CAR comprises an intracellular signaling domain that includes an immunoreceptor tyrosine-based activation motif (ITAM). Such an intracellular signaling domain may be from CD3z. In addition, the CAR construct may further comprise one or more costimulatory signaling domains, which may be from a co-stimulatory receptor, for example, from 4-1BB (CD137), CD28, CD40, OX40, or ICOS. The CAR construct disclosed herein may further comprise a transmembrane-hinge domain, which can be obtained from a suitable cell-surface receptor, for example, CD28 or CD8. The intracellular signal domain transmits the signals necessary for exertion of the effector function of the T or NK cell. More specifically, when the extracellular domain binds with the target ECM peptide, an intracellular signal domain transmits the signals necessary for activation of the cells. The intracellular signal domain includes the domain for transmitting the signals through for instance the TCR complex, and the domain for transmitting the costimulatory signals. Examples of the costimulatory molecule include CD28, 4-1BB (CD137), CD2, CD4, CD5, CD134, OX-40, and ICOS. A leader sequence or signal peptide may also be used to promote CAR secretion. For example, the leader sequence of the GM-CSF receptor may be used. In addition, the structure is preferably composed of an extracellular domain and a transmembrane domain linked together through a spacer domain. More specifically, the CAR according to a preferred embodiment contains a spacer domain between the extracellular domain and transmembrane domain.

Also provided are isolated nucleic acid molecules and vectors encoding any of the anti-Tn CARs as disclosed herein, and host cells, such as host immune cells (e.g., T cells and natural killer cells), comprising the nucleic acid molecules or vectors. Immune cells expressing anti-CTLA-4 CARs, which comprises a Tn-specific antibody binding fragment, can be used for the treatment of diseases mediated by Tn-antigen+ cells.

Preferably, the Tn-specific CAR-expressing T and NK cells have a lower affinity for the Tn antigen of normal origin (e.g., Tn antigen on circulating human IgA1), compared with their affinity for Tn antigen associated with cancer. It is preferred that the higher affinity of the CAR-T or NK cells for the cancer Tn-antigen is at least 10%, at least 20%, at least 50%, at least 60%, at least 70%, at least 80%, at least 90%, at least 95% or at least 100%, compared with the affinity of the CAR-T or NK cell for the Tn-antigen of normal origin (e.g., Tn antigen on circulating human IgA1). It is preferred that the decrease in affinity of the CAR-T or NK cell for Tn-antigen of normal origin (e.g., Tn antigen on circulating human IgA1) is at least 10%, at least 20%, at least 50%, at least 60%, at least 70%, at least 80%, at least 90%, at least 95% or at least 100%, compared with the affinity of the CAR-T or NK cell for the cancer Tn antigen. It is preferred that the increase in affinity of the CAR-T or NK cell for the cancer ECM is at least 1.5-fold, at least 2-fold, at least 5-fold, at least 10-fold, at least 100-fold, more preferably at least 1000-fold greater than the affinity of the CAR-T or NK cell for the Tn-antigen of normal origin (e.g., Tn antigen on circulating human IgA1).

### VI. Diagnostic Applications

Any of the anti-Tn antibodies disclosed herein can also be used for detecting presence of Tn antigen (e.g., Tn antigen on cancer cells) in vitro or in vivo. Results obtained from such detection methods can be used for diagnostic purposes (e.g., diagnosing diseases associated with Tn antigen) or for scientific research purposes (e.g., identifying new Tn antigen expressing cell types, studying bioactivity and/or regulation of Tn antigen). For assay uses such as diagnostic uses, an anti-Tn antibody as described herein may be conjugated with a detectable label (e.g., an imaging agent such as a contrast agent) for detecting presence of Tn antigen (e.g., Tn antigen on cancer cells), either in vivo or in vitro. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker.

Conjugated or attached can include covalent or noncovalent bonding as well as other forms of association, such as entrapment, e.g., of one entity on or within the other, or of either or both entities on or within a third entity, such as a micelle.

In one example, an anti-Tn antibody as described herein can be attached to a detectable label, which is a compound that is capable of releasing a detectable signal, either directly or indirectly, such that the aptamer can be detected, measured, and/or qualified, in vitro or in vivo. Examples of such “detectable labels” are intended to include, but are not limited to, fluorescent labels, chemiluminescent labels, colorimetric labels, enzymatic markers, radioactive isotopes, and affinity tags such as biotin. Such labels can be conjugated to the aptamer, directly or indirectly, by conventional methods.

In some embodiments, the detectable label is an agent suitable for detecting Tn-antigen expressing cells in vitro, which can be a radioactive molecule, a radiopharmaceutical, or an iron oxide particle. Radioactive molecules suitable for in vivo imaging include, but are not limited to, group consisting of fluorine-18, zirconium-89, copper-64, yttrium-86, indium-111, ¹²²I, ¹²³I, ¹²⁴I, ¹²⁵I, ¹³¹I, ¹⁸F, ⁷⁵Br, ⁷⁶Br, ⁷⁷Br, ²¹¹At, ²²⁵Ac, ¹⁷⁷Lu, ¹⁵³Sm, ¹⁸⁶Re, ¹⁸⁸Re, ⁶⁷Cu, ²¹³Bi, ²¹²Bi, ²¹²Pb, and ⁶⁷Ga. Exemplary radiopharmaceuticals suitable for in vivo imaging include ¹¹¹In Oxyquinoline, ¹³¹I Sodium iodide, ^{99m}Tc Mebrofenin, and ^{99m}Tc Red Blood Cells, ¹²³I Sodium iodide, ^{99m}Tc Exametazime, ^{99m}Tc Macroaggregate Albumin, ^{99m}Tc Medronate, ^{99m}Tc Mertiatide, ^{99m}Tc Oxidronate, ^{99m}Tc Pentetate, ^{99m}Tc Pertechnetate, ^{99m}Tc Sestamibi, ^{99m}Tc Sulfur Colloid, ^{99m}Tc Tetrofosmin, Thallium-201, or Xenon-133.

The detectable agent can also be a dye, e.g., a fluorophore, which is useful in detecting a disease mediated by Tn antigen secreting cells in tissue samples.

To perform a diagnostic assay in vitro, an anti-Tn antibody can be brought in contact with a biological sample obtained from a subject suspected of containing Tn antigen, e.g., Tn-antigen expressing cancer cells. The antibody and the sample may be incubated under suitable conditions for a suitable period to allow for binding of the antibody to the Tn antigen. Such an interaction can then be detected via routine methods, e.g., ELISA, histological staining or FACS. A biological sample, as used herein, refers to samples obtained from a subject. Non-limiting examples of a biological sample can be blood, urine, stool, tissue biopsy, or any other bodily fluids.

To perform a diagnostic assay in vivo, a suitable amount of anti-Tn antibodies, conjugated with a label (e.g., an imaging agent or a contrast agent), can be administered to a subject in need of the examination. Presence of the labeled antibody can be detected based on the signal released from the label by routine methods.

To perform scientific research assays, an anti-Tn antibody can be used to study bioactivity of Tn antigen, and/or detect the presence of Tn antigen intracellularly. For example, a suitable amount of anti-Tn antibody can be brought in contact with a sample (e.g., a new cell type that is not previously identified as Tn antigen expressing cells) suspected of expressing Tn antigen. The cells are permeabilized prior to contacting the anti-Tn antibody. The antibody and the sample may be incubated under suitable conditions for a suitable period to allow for binding of the antibody to the Tn antigen. Such an interaction can then be detected via routine methods, e.g., ELISA, histological staining or FACS.

### VII. Kits for Therapeutic and Diagnostic Applications

The present disclosure also provides kits for the therapeutic or diagnostic applications as disclosed herein. Such kits can include one or more containers comprising an anti-Tn antibody, e.g., any of those described herein.

In some embodiments, the kit can comprise instructions for use in accordance with any of the methods described herein. The included instructions can comprise a description of administration of the anti-Tn antibody to treat, delay the onset, or alleviate a target disease as those described herein. The kit may further comprise a description of selecting an individual suitable for treatment based on identifying whether that individual has the target disease. In still other embodiments, the instructions comprise a description of administering an antibody to an individual at risk of the target disease.

The instructions relating to the use of an anti-Tn antibody generally include information as to dosage, dosing schedule, and route of administration for the intended treatment. The containers may be unit doses, bulk packages (e.g., multi-dose packages) or subunit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder treatable by modulating immune responses, such as autoimmune diseases. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like.

Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-Tn antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

Also provided herein are kits for use in detecting Tn antigen in a sample. Such a kit may comprise any of the anti-Tn antibodies described herein. In some instances, the anti-Tn antibody can be conjugated with a detectable label as those described herein. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

Alternatively or in addition, the kit may comprise a secondary antibody capable of binding to anti-Tn antibody. The kit may further comprise instructions for using the anti-Tn antibody for detecting Tn antigen.

### VIII. General Techniques

The practice of the present invention will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Novel Recombinant Anti-Tn IgG and IgM as Tumor Diagnostic Biomarkers

The Tn antigen is a neoantigen abnormally expressed in many human carcinomas. It correlates with metastasis and poor survival. To explore its biomarker potential, two recombinant antibodies to the Tn antigen were generated—one is a chimeric human IgG1 antibody Remab6 and the other is a murine IgM antibody ReBags6. Their specificities were characterized using multiple biochemical and biological approaches. Both Remab6 and ReBags6 recognize clustered Tn structures but importantly do not recognize glycoforms of human IgA1 that contains potential cross-reactive Tn antigen structures. In flow cytometry and immunofluorescence analyses, Remab6 recognizes many human cancer cell lines expressing the Tn antigen, but not their Tn-negative counterparts. In immunohistochemistry (IHC), Remab6 stains many human cancers in tissue array format but rarely stains in normal tissues. Proteomic approaches were used to identify several unique Tn containing glycoproteins in the Colo205 simple cell line (Tn-positive). Thus, Remab6 is useful for biochemical characterization of cancer cell lines and IHC of tumor sections, and represent promising tools for Tn biomarker discovery independently of recognition of IgA1.

### Introduction

Altered glycosylation is a hallmark of cancer and aberrant glycan structures, or tumor-associated carbohydrate antigens (TACAs), are biomarkers of tumor progression (1,2). A highly relevant TACA is the Tn antigen (GalNAc□1-Ser/Thr), which is a truncated form of O-GalNAc mucin-type O-glycans. The expression of the Tn antigen and sialylTn (STn) in tumors represent potential markers associated with poor prognosis and tumor metastasis (3-7). The Tn antigen is a biosynthetic precursor to all extended O-GalNAc glycans in human cell glycoproteins and is generated by one of twenty polypeptide N-acetylgalactosaminyltransferases (ppGalNAc-Ts) (8). In normal tissues, the Tn antigen is not expressed on mature surface membrane glycoproteins, as it is efficiently modified by a single enzyme, the T-synthase (encoded by C1GALT1), which in the Golgi apparatus transfers a galactose residue onto Tn to form the core 1 O-glycan structure (Galβ1-3GalNAcα1-Ser/Thr) that can be further modified with other sugars (9,10). The biosynthesis of active T-synthase requires a specific molecular chaperone, Cosmc (encoded by the X-linked C1GALT1C1) (10), which functions in the endoplasmic reticulum to assist folding and activity of the T-synthase. Importantly, acquired mutations or alterations in Cosmc expression have been associated with many human cancers, including pancreatic cancer, where the Tn antigen is typically highly expressed (11-13).

In many studies, the expression of the Tn antigen is examined using plant lectins, but some use monoclonal antibodies, whose specificities may not be limited to the Tn antigen (5,14-17). Another complication is that many anti-Tn reagents recognize terminal □-linked GalNAc residues, and could possibly bind to that residue in blood group A (BGA), Forssman-related antigens, and IgA1. The latter is a confounding factor to the potential use of the Tn antigen as a tumor biomarker, as circulating glycoforms of human IgA1 contain the Tn antigen in the hinge region (18). Thus, any antibody that binds the Tn antigen and also Tn-positive IgA1 glycoforms would have limited usefulness. Furthermore, while several potentially Tn-positive glycoproteins, such as mucins CA15-3 (MUC1) and CA125 (MUC16) have been developed for clinical use to follow cancer progression (19,20), the expression and identification of the mucin protein epitopes are not specific enough for use in cancer diagnosis. As the Tn antigen might be present in several tumor-specific Tn-positive glycoproteins released into plasma, ongoing studies are aimed at generating specific antibodies with restricted specificity to the Tn antigen on glycoproteins and lacking recognition of IgA1.

To this end, a novel, recombinant human chimeric IgG1 anti-Tn antibody, named Remab6, derived in part from an original murine anti-Tn monoclonal antibody (Ca3638, BaGs6) (21) was generated. A recombinant murine IgM of this antibody, ReBaGs6, which allows direct comparison of the experimental results obtained with the ascites to those obtained with the recombinant ReBaGs6, was also generated. The results show that these antibodies have high affinity and are remarkably specific for the Tn antigen with no recognition of human IgA1, and are useful in immunohistochemistry (IHC) with a variety of human cancers. Thus, these antibodies represent new useful reagents to detect Tn-positive glycoproteins as a biomarker for human carcinomas and may also be a novel therapeutic agent for targeted cancer treatment.

### (i) Materials and Methods

### Cell Culture

Human colorectal carcinoma LSC and LSB cells were a kind gift from Dr. Steven Itzkowitz (Mount Sinai School of Medicine) (22). Human colorectal carcinoma LS174T, and acute T cell leukemia Jurkat cells, which carry a Cosmc mutation and express the Tn antigen (10), were purchased from American Type Culture Collection (ATCC). Tn-positive and -negative populations in LS174T cells were isolated previously (11). Human colon adenocarcinoma Colo205 cell line, human breast adenocarcinoma MDA-MB-231 cell line, human gastric adenocarcinoma MKN-45 cell line, and the corresponding Tn-positive Simple Cells of each cell line, which were generated by deletion of the Cosmc gene, were a kind gift from Dr. Henrik Clausen (University of Copenhagen) (23). LSC, LSB, LS174T, and MDA-MB-231 cells were cultured in Dulbecco's Modified Eagle's Medium (DMEM) (Corning®) supplemented with 10% (vol/vol) fetal bovine serum and 200 units/ml penicillin-streptomycin at 37° C. and 5% CO2. Jurkat, Colo205 and MKN-45 cells were cultured in RPMI 1640 medium (Corning®) supplemented with 10% (vol/vol) fetal bovine serum and 200 units/ml penicillin-streptomycin at 37° C. and 5% CO2.

### Preparation of Asialo-BSM Affinity Resin

Bovine submaxillary mucin (BSM) (Sigma) was coupled with UltraLink™ support (Thermo Fisher Scientific) as previously described (22). The coupled BSM beads were desialylated by the addition of 50 mU of neuraminidases (Roche) in 50 mM sodium acetate (pH 5.0) for 1 h at 37° C. The resin was washed with PBS three times, and the beads were collected and desialylated once more to completely remove the sialic acid, to generate desialylated BSM (Asialo-BSM) beads.

### Immunofluorescence of Asialo-BSM Beads

BSM beads, Asialo-BSM beads or beads alone were incubated with anti-Tn ascites (BaGs6; IgM, diluted 1:100 in PBS) or mouse anti-STn mAb (SCBT, B72.3, diluted to 1 g/ml in PBS) for 1 h on ice. Then the beads were washed 3 times with 1M NaCl, and incubated with Alexa Fluor 488-goat anti-mouse IgM or IgG (Thermo Fisher Scientific) at 1:400 dilution in PBS for 1 h on ice in the dark. The beads were washed 3 times with PBS, and analyzed using a microscope (Zeiss; Axioimager M1). Isotype antibodies, mouse IgM and IgG, were used as controls, for the ascites and anti-STn mAb, respectively.

Affinity Purification and Sequencing of Anti-Tn Antibody from Mouse Ascites

An ascites fluid containing BaGs6 (8 mg/ml), a murine IgM monoclonal antibody, was a kind gift from the late Georg Springer, but no hybridoma exists. To purify the BaGs6, the ascites fluid was mixed with Asialo-BSM beads and rotated overnight in cold room. After the mixture was washed 6 times with 1M NaCl on the column, bound material was eluted by 0.1 M glycine-NaOH (pH 10.5) and neutralized with 1M glycine-HCl (pH 2.7). The concentration of purified mAb was determined by Pierce™ BCA assay kit (Thermo Fisher Scientific) following the manufacturer's instructions with BSA as a standard. Each fraction was analyzed on SDS-PAGE (Genscript), and stained with Coomassie. The affinity purified antibody was de novo sequenced by LC-MS/MSn with multiple proteolytic approaches at Digital Proteomics Inc. (San Diego, CA). Using this information, genes encoding the heavy and light chain and complementarity determining regions (CDR) with appropriate proprietary amino acid substitutions were constructed. For large scale production, ReBaGs6 was outsourced to LakePharma Inc. (San Carlo, CA) for production and purification.

Cloning and Expression of Recombinant Human Chimeric Anti-Tn mAb (Remab6, hIgG₁)

DNA fragments engineered to represent the heavy mu chain and kappa light chain variable domains in BaGs6 were synthesized by Genewiz, Inc. (South Plainfield, NJ) into pUC57 vector. Synthetic DNA fragments representing aspects of the variable domains of BaGs6 were subcloned into pFUSEss-CHIg-hG1 and pFUSE2ss-CLIg-hk vector (InvivoGen), respectively. HEK293 Freestyle cells were grown to a density of 2.5×106 cells/ml in Freestyle Expression Medium (Life Technologies) in suspension on a platform shaker in a humidified 37° C. incubator. Before transfection, the cells were harvested at 300×g for 10 min, and resuspended in fresh medium. Then the cells were co-transfected with 3 μg/ml in total of plasmid vectors expressing heavy chain and light chain (2:3 ratio), and with 9 μl/ml of polyethylenimine (PEI) at a final concentration of 0.5 μg DNAs or PEI/μl media in transfection solution. After 24 h, the cells were diluted 1:1 with fresh media supplemented with valproic acid (Sigma Aldrich) at a final concentration of 2.2 mM. After 7 days post-transfection, cultured supernatant was collected after centrifugation at 300×g for 10 min.

### Purification of Remab6 Using Protein a Affinity Chromatography

The culture supernatant was applied to a Protein A-Agarose column (Roche) equilibrated with 100 mM Tris, pH 8.0. After the column was washed with 100 mM Tris-HCl, pH 8.0, once, and 10 mM Tris-HCl, pH 8.0, twice, Remab6 was eluted by 0.1 M glycine-HCl, pH 2.7, and neutralized with 1 M Tris-HCl, pH 9. The concentration of purified Remab6 was determined with the Pierce™ BCA assay kit.

### Tn Glycopeptide Microarray and CFG Microarray

The Tn glycopeptide microarray was prepared as previously described (25). The list of glycopeptides printed on the microarray is given in Table 1. The Consortium for Functional Glycomics (CFG) glycan microarray version 5.0 was used (www.functionalglycomics.org) (26). Briefly, Remab6 (diluted to 5 μg/ml), ReBaGs6 (diluted to 20 and 2 μg/ml) or mouse ascites (diluted 1:200) in TSM binding buffer (20 mM Tris-HCl, pH 7.4, 150 mM NaCl, 2 mM CaCl₂), 2 mM MgCl2, with 1% BSA and 0.05% Tween-20) were added to the array slides for 1 h at RT. Slides were washed four times with TSM wash buffer 1 (20 mM Tris-HCl, pH 7.4, 150 mM NaCl, 2 mM CaCl₂), 2 mM MgCl2 and 0.05% Tween-20), and washed four times with TSM wash buffer 2 (20 mM Tris-HCl, pH 7.4, 150 mM NaCl, 2 mM CaCl2) and 2 mM MgCl2). Alexa Fluor® 488-labeled goat anti-human IgG or goat anti-mouse IgM secondary (diluted to 5 μg/ml) were used for detection. After washing as above, slides were washed once in water and dried before being read on a Genepix® 4300A microarray scanner (Molecular Devices). Images were analyzed with quantitation software (GenePix® Pro Microarray Analysis Software Ver. 7, Molecular Devices).

### Preparation of Remodeled Glycopeptide Arrays—STn and T Glycopeptide Array

The STn glycopeptide array was generated by incubating the Tn glycopeptide microarray slides (ID1-8) with the sialyltransferase, ST6GalNAc-I, and 1 mM of 5′-biotinylated CMP-Neu5Ac (Chemily, LLC, custom order) in reaction buffer (150 mM NaCl, 20 mM cacodylate, pH 6.8, 10 mM CaCl₂), 10 mM MgCl2, 10 mM MnCl2 and 0.05% Tween-20) at 37° C. overnight. Slides were washed four times with TSM wash buffer 1 and TSM wash buffer 2 before use. The T glycopeptide array was generated by incubating the Tn glycopeptide microarray slide with T-synthase with 1 mM of UDP-Gal in reaction buffer at 37° C. overnight. Slides were washed four times with TSM wash buffer 1 and TSM wash buffer 2, and microarrays were probed with Remab6.

Preparation of IgA from Cosmc-KO Dakiki Cells and Cell Extracts

Cosmc-KO Dakiki cells were generated using the CRISPR/Cas9 system, which allowed us to delete the functional Cosmc gene on the X-chromosome. Purification of IgA1 from Dakiki cells was previously described (18). For cell extracts, approximately, 5×106 cells were harvested and lysed with 500 μl of lysis buffer (150 mM NaCl, 20 mM Tris-HCl, pH 7.5, 2.5 mM sodium pyrophosphate, 1 mM Na2EDTA, 1 mM EGTA, 1 mM β-glycerophosphate, 1 mM sodium orthovanadate and 1% Triton-X100) containing protease inhibitors (Roche, cOmplete™, Mini Protease Inhibitor Cocktail). After sonication, cell extract was collected from the supernatant after centrifugation at 15,000 rpm for 10 min.

### Western and Lectin Blots

The protein concentration in purified IgA1 and cell extracts was determined by Pierce BCA kit. Asialo-BSM was prepared from BSM with neuraminidases as described in “Preparation of Asialo-BSM affinity resin”. Proteins (purified IgA; 0.3 μg/lane, cell extracts; g/lane, and Asialo-BSM; 2.5 μg/lane) were analyzed on SDS-PAGE gel (Genscript), and transferred to a nitrocellulose membrane (Thermo Fisher Scientific). After blocking with 5% (w/vol) BSA in TBS+0.05% Tween-20 (TTBS) for 1 h at RT, Western and lectin blots were analyzed with Remab6 and ReBaGs6 (diluted at 5 μg/ml, 2 μg/ml in TTBS, respectively), biotinylated VVA (Vector Laboratories, diluted at 1 μg/ml in TTBS) or horseradish peroxidase (HRP)-labeled goat anti-human IgA (alpha) antibody (Sigma, diluted at 1:5000 in TTBS) as a primary staining. Secondary detection was performed with horseradish peroxidase (HRP)-labeled goat anti-human IgG antibody, goat anti-mouse IgM antibody (KPL), and streptavidin-HRP (Vector Laboratories) at 1:5000 dilution in TTBS, using SuperSignal™ West Pico Chemiluminescent Substrate (Thermo Fisher Scientific), then analyzed on an Amersham™ Imager 600 (GE Healthcare Life Sciences).

### Binding and Inhibition Assays

Asialo-BSM (0.5 μg/well) was immobilized with immobilization buffer (NaHCO₃/Na2CO3, pH 9.6) in 96-well plate (Thermo Fisher Scientific, PolySorp) overnight at 4° C. The plate was washed with TTBS, and added with 5% (w/v) BSA in TTBS for 1 h at RT. The plate was incubated with a serial dilution of ReBaGs6, Remab6, and biotinylated VVA in TTBS for 1 h at RT. The plate was washed with TTBS, and incubated with Alexa Fluor® 488-labeled goat anti-mouse IgM, goat anti-human IgG, or streptavidin at 1:1000 dilution in TTBS for 1 h at RT in the dark. The plate was washed with TTBS, and read on an ImageExpress® Pico (Molecular Devices). Affinity constant was calculated with GraphPad Prism 6.0 (GraphPad Software, Inc.). For inhibition assay, ReBaGs6, Remab6, and biotinylated VVA were preincubated with 100 mM GalNAc, or 100 mM GlcNAc for 30 min at RT.

### Flow Cytometry

Cells were collected, and washed with cold PBS twice, then 5×105 cells were transferred into tubes. Cells were treated with 50 mU of neuraminidases in PBS for 1 h at 37° C., then washed, and incubated with 100 μl of Remab6 (diluted to 5 μg/ml in PBS) or mouse anti-STn mAb (diluted to 1 μg/ml in PBS) for 1 h on ice. For controls, cells were incubated with 100 μl of human IgG or mouse IgG (SouthernBiotech) for 1 h on ice. Cells were washed twice with cold PBS, and incubated with 100 μl of Alexa Fluor® 488-labeled goat anti-human IgG or goat anti-mouse IgG (Thermo Fisher Scientific) at 1:400 dilution in PBS for 1 h on ice in the dark. Cells were washed twice with cold PBS, resuspended in 500 μl of PBS, and analyzed on a flow cytometer (FACSCalibur™, Becton Dickinson).

### Confocal Microscope Imaging

For immunofluorescence with cultured cells, cells were fixed with 4% (vol/vol) paraformaldehyde (PFA) in PBS for 30 min on ice, permeabilized with 0.1% (vol/vol) Triton X-100 in PBS for 10 min on ice. After blocking with 5% (w/vol) BSA in PBS for 1 h at 4° C., cells were co-stained with Remab6 (diluted to 5 μg/ml in PBS+0.5% BSA) and rabbit anti-Calnexin mAb (CST, 1:100 dilution in PBS+0.5% BSA), rabbit anti-Giantin pAb (Abcam, 1:500 dilution in PBS+0.5% BSA) or sheep anti-TGN46 pAb (Bio-Rad, 1:500 dilution in PBS+0.5% BSA) for 1 h at 4° C. Cells were washed 3 times with PBS, and co-stained with Alexa Fluor® 488-labeled goat anti-human IgG and Alexa Fluor® 568-labeled goat anti-rabbit IgG or Alexa Fluor® 568-labeled donkey anti-sheep IgG secondaries (Thermo Fisher Scientific) at 1:400 dilution in PBS+0.5% BSA for 1 h at 4° C. in the dark. Cells were washed 3 times with PBS, and stained with DAPI for 10 min at RT in the dark, then cells were analyzed by confocal microscope (Zeiss; Axioimager Z1). Isotype antibodies (human IgG, rabbit IgG or sheep IgG) (Southern Biotech) were co-stained as controls.

### Immunoprecipitation and Western Blot

Cell extracts were prepared as described in “Preparation of IgA1 from Cosmc-KO Dakiki cells and cell extracts”. Cell extracts from the Colo205 simple cell line (Tn-positive) were immunoprecipitated with ReBaGs6 or isotype control mouse IgM-conjugated with UltraLink™ beads overnight at 4° C. Immunoprecipitates were analyzed by Western blot as described in “Western and lectin blots”. Remab6 (2 μg/ml in TTBS), and horseradish peroxidase (HRP)-labeled with goat anti-human IgG antibody (KPL) at 1:5000 dilution in TTBS were used for detection.

### LC/ESI-MS/MS Analysis

Immunoprecipitates in Coomassie-stained gel were de-stained, and washed with a series of three washing buffers (50 mM ammonium bicarbonate, 50% acetonitrile, and 80% acetonitrile). The bound proteins were reduced with 1 ml of 40 mM dithiothreitol for 25 min at 56° C. The pieces of gel were rinsed with 1 ml of 50 mM ammonium bicarbonate buffer, and the reduced proteins were alkylated with 1 ml of 50 mM Iodoacetamide for 30 min at 25° C. in the dark with gentle mixing. The iodoacetamide was discarded, and the gel-bound proteins were digested with 0.5 ml of trypsin (Promega, diluted at 20 ng/μl) in 50 mM ammonium bicarbonate buffer at 37° C. with gentle mixing for 12 h. After digestion, the tryptic fractions were collected, and the gels were washed with 50 mM ammonium bicarbonate to collect any remaining tryptic peptides. The eluents containing tryptic peptides were dried using a Speed-Vac apparatus (Thermo Fisher Scientific), and stored at 4° C. prior to mass spectrometric analysis. The dried samples were dissolved with 20 μl of 0.1% formic acid/water. Two μl of each sample was analyzed by LC/ESI-MS/MS using a Fusion™ Lumos™ (Thermo Fisher) mass spectrometer (MS) with a Dionex LC system using data dependent acquisition with dynamic exclusion (DE=1) settings. The data dependent acquisition settings were a top 12 higher energy collision induced dissociation (HCD) for the Fusion™ Lumos™ MS. Resolving power for Fusion™ Lumos™ was set at 120,000 for the full MS scan, and 35,000 for the MS/MS scan at m/z 200. LC/ESI-MS/MS analyses were conducted using a C18 column (75 μm×150 m). The mobile phases for the reverse phase chromatography were (A) 0.1% HCOOH/water, and (B) 0.1% HCOOH in acetonitrile. A four-step of linear gradient was used for the LC separation (2% to 30% B in the first 47 min, followed by 80% B in the next 1 min, and holding at 80% B for 12 min). The SEQUEST algorithm was used to identify peptides from the resulting MS/MS spectra by searching against the combined human protein database (22,673 proteins in total) extracted from Swiss-prot (version 57) using taxonomy “Homo sapiens” using Proteome Discoverer™ (Thermo Scientific, version 1.4). Searching parameters for parent and fragment ion tolerances were set as 15 ppm and 30 mmu for the Fusion™ Lumos™. Other parameters were used as a fixed modification of carbamidomethylation (C), variable modifications of acetylation (K), HexNAc (S, T) and oxidation (Met). Trypsin was set as the protease with a maximum of 2 missed cleavages. Raw files were searched against top 6 O-glycans using Byonic™ (27) with a peptide tolerance of 15 ppm; an MS/MS tolerance of 20 ppm for HCD data; the carbamidomethylated cysteine as fixed modification and oxidation of methionine and acetylation (K) as variable modifications. Byonic™ scoring gives an indication of whether modifications are confidently localized.

### Immunohistochemical Staining in IEC-Cosmc KO Mice Sections and Human Cancer Cell Blocks

Intestine from WT and IEC-Cosmc KO mice were dissected and prepared by swiss-roll as previously described (28), following all approved IACUC protocols and guidelines. Formalin-fixed paraffin-embedded (FFPE) sections were deparaffinized, and retrieved by boiling for 10 min in retrieval buffer (10 mM Citrate, pH 6). After cooling down, sections were treated with 0.3% H₂O₂ for 10 min and blocked with 5% (vol/vol) goat serum (Gibco) in TBS for 1 h at RT. Sections were stained with Remab6 (5 g/ml in TBS+0.1% Triton X-100, TBSTx) overnight at 4° C. HRP-labeled goat anti-human IgG antibody (KPL) at 1:400 dilution in TBSTx was added for 1 h at RT, then staining was visualized using AEC single solution (Invitrogen), which generates a brownish-red deposit where binding occurs in the tissue. Nuclear staining was performed with hematoxylin. Images were analyzed by microscope (Zeiss; Axioimager M1). Isotype human IgG was used as a control.

For human cancer cell block sections, harvested cells were collected, and suspended in 10 ml of 10% neutral buffered formalin (Midland Scientific Inc.) overnight at RT. Cell pellets were suspended in 1% warm agarose solution, and added to respective Eppendorf tubes with agar plug. After hardening of the agar for 15 min, agar with cells was fixed again in 10% neutral buffered formalin overnight at 4° C. Agar with cells was extruded into the immersed cassette by cutting off the bottom of the tube with a razor blade, then paraffinized.

Preparation of Remab6-Fab and Conjugation with HRP

Remab6 was cleaved with papain enzyme and prepared following the manufacturer's instructions (Thermo Fisher Scientific). Conjugation with HRP to Remab6-Fab was prepared following the manufacturer's instructions (Abcam). Remab6-Fab-HRP binding to IEC-Cosmc KO and WT tissues was assessed, and as a control, isotype-matched human IgG-Fab-HRP was prepared and used for the assay.

### Immunohistochemical Staining in Human Cancer Tissue Array

Tissue array sides (FDA808k-1, and k-2) were purchased from US Biomax Inc. The list of tissues is given in Table 2.1 and 2.2. Immunohistochemical staining with Remab6-Fab-HRP was performed as described in “Immunohistochemical staining”. Isotype human IgG-Fab-HRP was used as a control

### (ii) Results

Affinity Purification of the Tn-Reactive IgM from BaGs6 Ascites

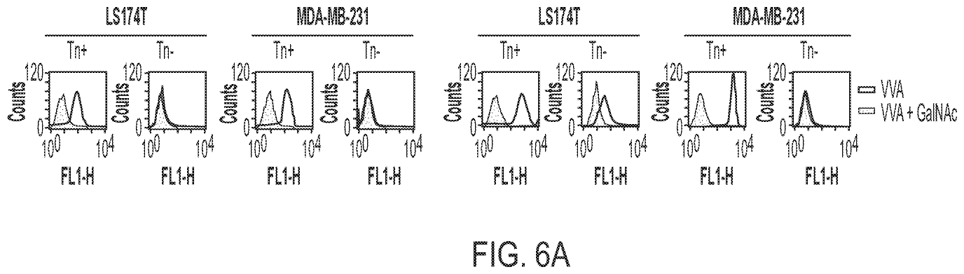

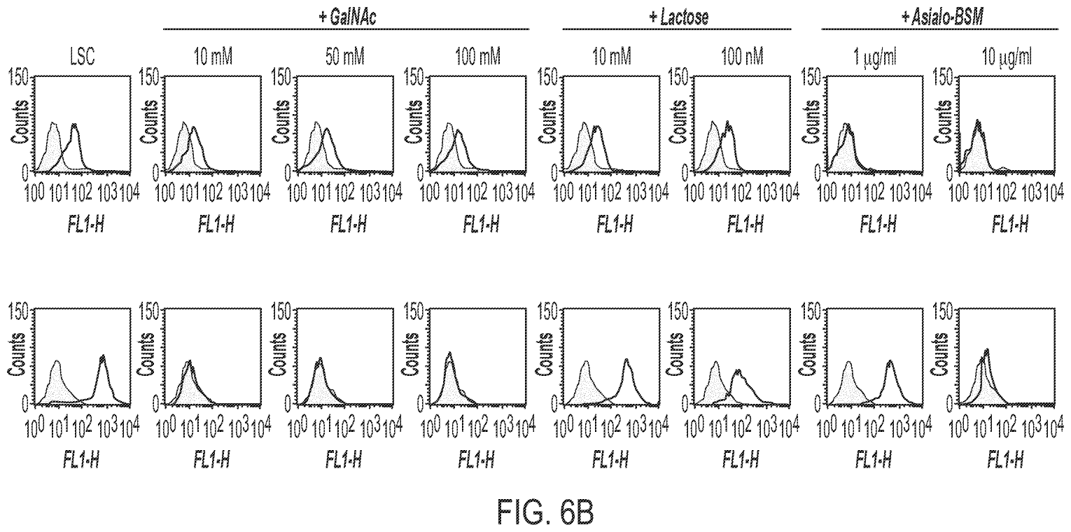

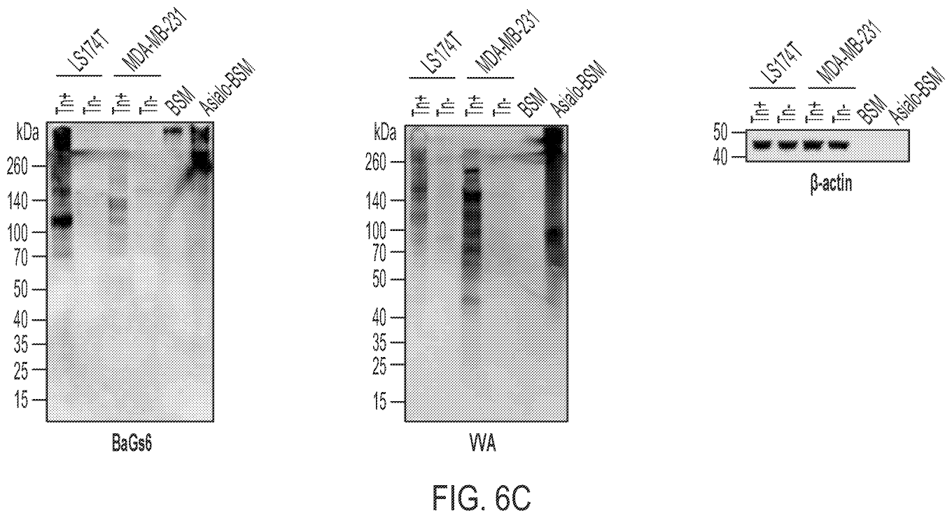

BaGs6 is a murine IgM arising from immunization with Tn-positive cells and available only as a mouse ascites fluid (21). Previous microarray studies demonstrated that among all types of potential anti-Tn monoclonal antibodies that were screened, BaGs6 was the most specific and recognized di- and tri-Tn clustered structures on mucin glycopeptides (25). Flow cytometry data also demonstrated that BaGs6 specifically interacts with Tn-positive LS174T and MDA-MB-231 cells, similarly to the lectin Vicia villosa agglutinin (VVA), which indiscriminately recognizes terminal α-linked GalNAc on most types of glycoconjugates (29) (FIG. 6A). However, unlike VVA, binding of BaGs6 was incompletely inhibited by GalNAc, but was efficiently inhibited by Asialo-BSM, a mucin that contains a high density of Tn sites (FIG. 6B) (30). Although VVA was also inhibited by Asialo-BSM, inhibition was only observed at the highest concentration tested. Neither BaGs6 nor VVA were inhibited by lactose. A Western blot showed that both BaGs6 and VVA bind to multiple species with different molecular masses in Tn-positive cells (FIG. 6C). This demonstrates that BaGs6 and VVA differ in their recognition of glycoproteins.

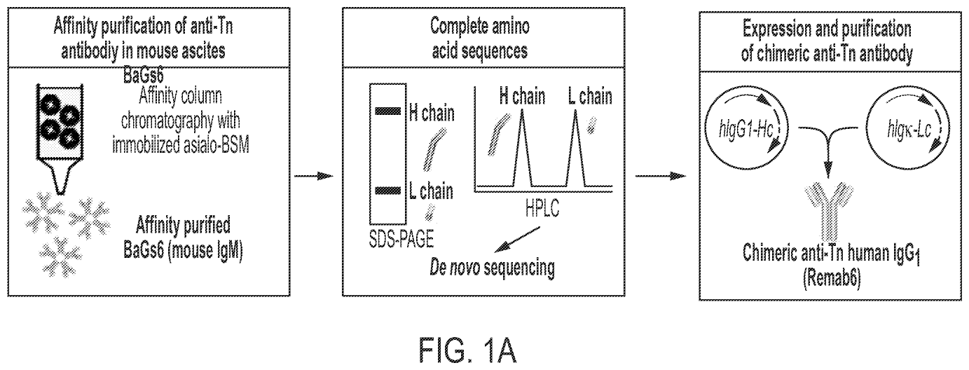

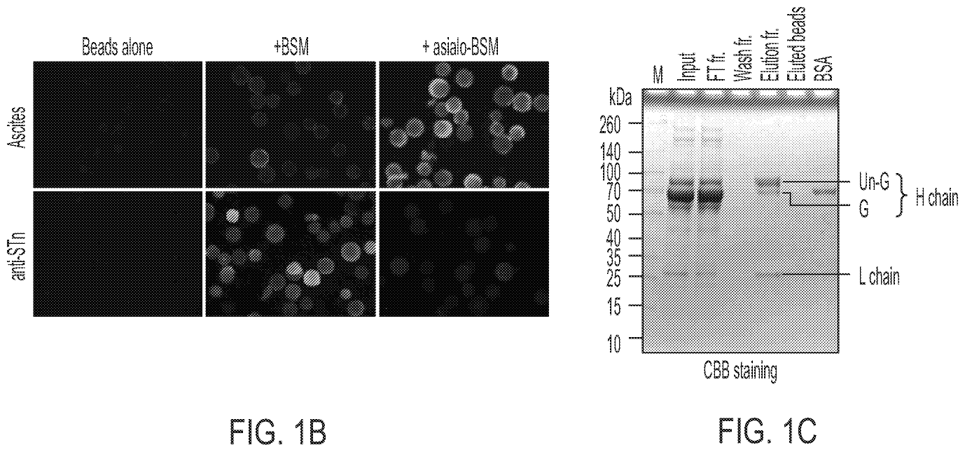

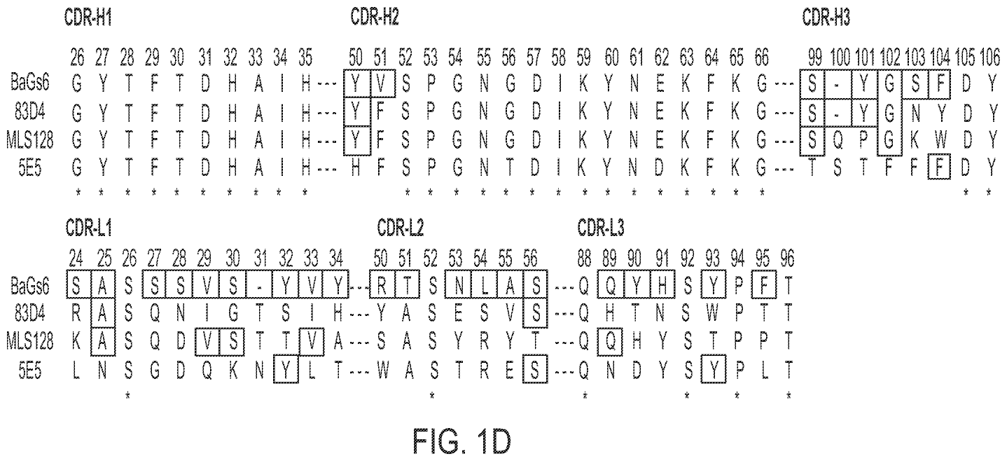

To isolate BaGs6 from the ascites fluid, Asialo-BSM was conjugated at highest densities to UltraLink beads to generate a high avidity Tn-rich resin (FIG. 1A). The ability of the affinity resin to bind BaGs6 was characterized by fluorescence microscopy, which demonstrated that the ascites-derived BaGs6 strongly interacted with Asialo-BSM resin, little with BSM resin, and did not interact with the unconjugated control beads (as detected by anti-IgM secondary antibody alone) (FIG. 1B). BaGs6 was successfully affinity-purified from the ascitic fluid and appeared homogeneous by Coomassie staining (FIG. 1C). The affinity purified BaGs6 was sequenced de novo using LC-MS/MS to generate a predicted amino acid sequence. Because of the uncertainty regarding the presence of leucine/isoleucine and some other residues, a recombinant antibody with an appropriately chosen amino acid sequence was engineered; the sequence for the complementarity determining regions (CDRs) within its variable regions are shown in FIG. 1D. The CDRs of BaGs6 were compared to those in three other known anti-Tn antibodies, 83D4 (31), MLS128 (32), and 5E5 (33). CDR3 is generally considered to be an essential region to determine the specificity of an antibody (34). Interestingly, CDR3 in heavy chain and CDR1-3 in light chain of BaGs6 exhibited significant diversity from the other available sequenced anti-Tn antibodies (FIG. 1D), suggesting that the binding specificity of BaGs6 will be somewhat different from the other antibodies.

### Generation of Remab6, a Chimeric Anti-Tn IgG, and ReBaGs6, a Mouse Anti-Tn IgM

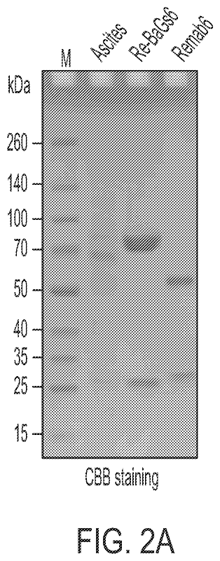

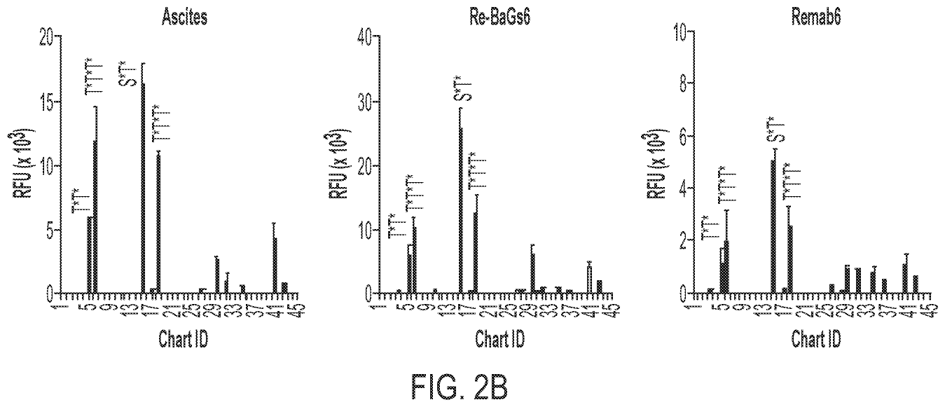

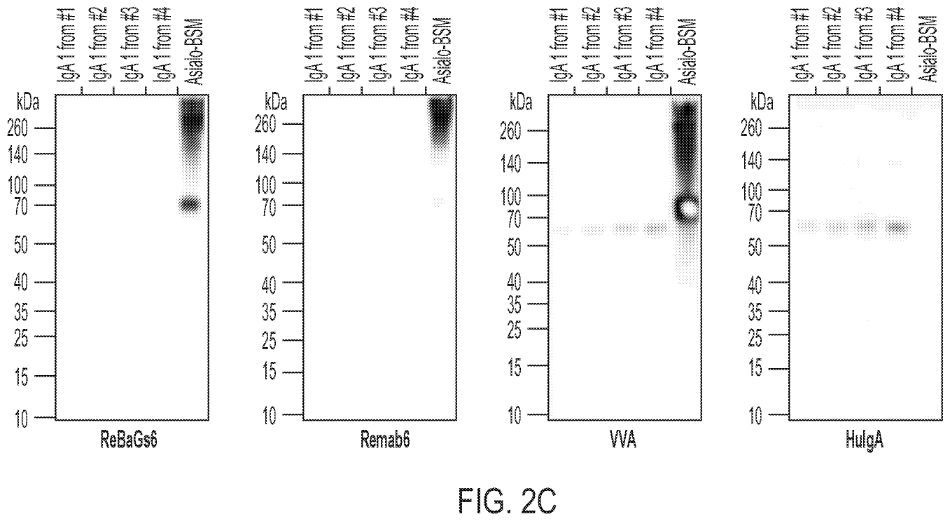

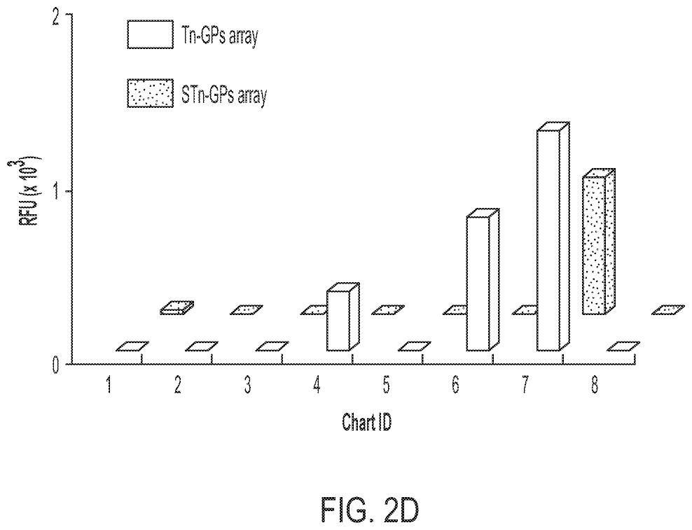

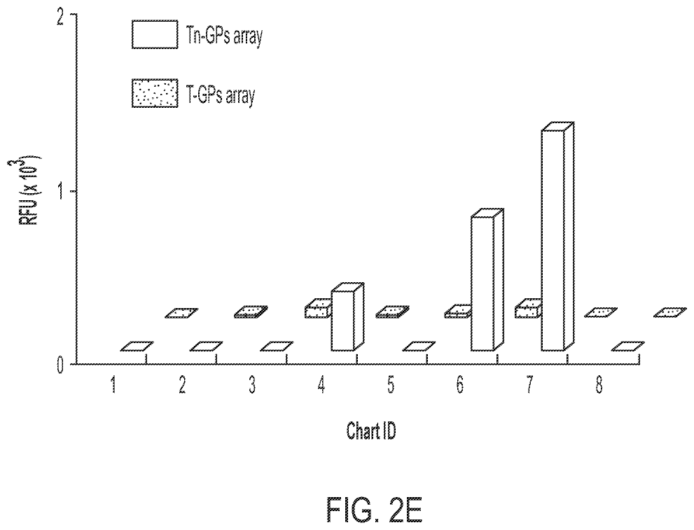

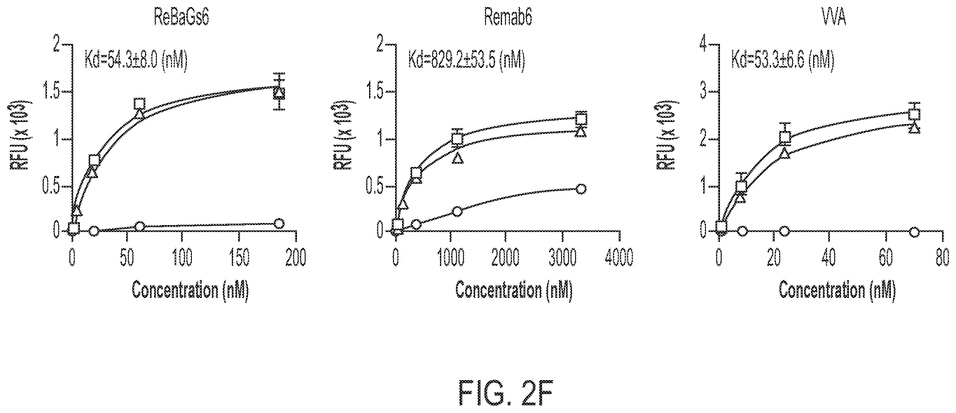

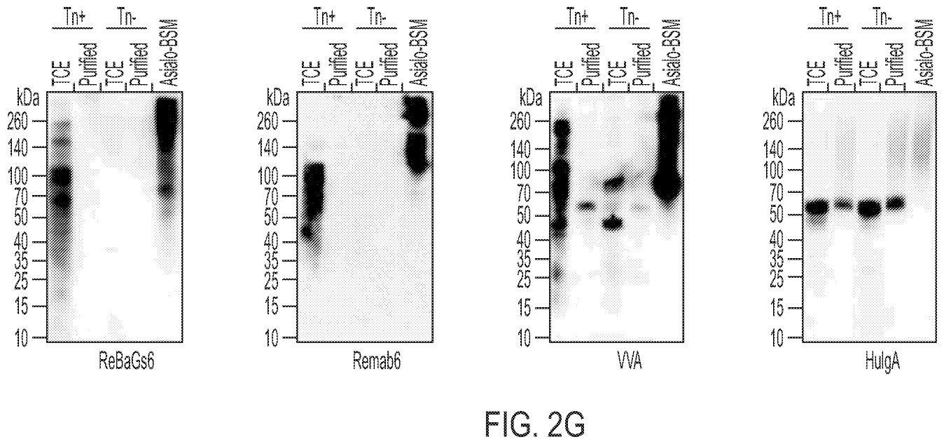

Using the sequence information, a recombinant antibody designated Remab6 was generated. Remab6 is a chimeric IgG1 and contains partial sequences of the mouse variable region from BaGs6 and the human constant region of IgG1. IgG1 constant region was selected due to its high utility in a number of biochemical assays, as well as ease of analysis and coupling to Protein A/G-Sepharose. It is also specifically recognized by common anti-human Ig reagents, making it useful in murine studies where no interference with murine Ig would occur. Furthermore, a recombinant BaGs6 (ReBaGs6)—a murine IgM (mIgM)—was also generated, ReBaGs6 includes the mouse variable region and the mouse constant region of IgM. Both Remab6 and ReBaGs6 were expressed in human HEK293 cells and purified (FIG. 2A).

### Microarray Studies of Remab6 and ReBaGs6 Show Specificity to a Subset of Tn Glycopeptides

The specificity of Remab6 and ReBaGs6 were compared using a Tn glycopeptide microarray (Table 1), which contains many peptides with one or more Tn antigens, including peptides modeled after the hinge region of human IgA1. These antibodies showed similar recognition patterns to that of the original mouse ascites (FIG. 2B). Both Remab6 and ReBaGs6 recognized di- and tri-Tn clustered structures on mucin-derived glycopeptides, but only weakly interacted with synthetic glycopeptides containing the Tn antigen on IgA1 (FIG. 2B). As circulating IgA1 has two distinct glycoforms as previously described (Helix pomatia agglutinin (HPA)+/Peanut agglutinin (PNA)−, which are Tn/STn-positive; HPA−/PNA+, which represent extended O-glycans) (18), whether either Remab6 and ReBaGs6 bound to Tn antigen on IgA1 were tested on Dakiki B cells, in which the Cosmc gene was deleted and thus express Tn antigen on the cell surface as well as on their secreted IgA1. IgA1 in total cell extracts was examined for its binding to Remab6, ReBaGs6 and VVA. Consistent with Tn glycopeptides array results, both Remab6 and ReBaGs6 bound only to Tn-expressing glycoproteins in Cosmc KO Dakiki cell extracts, but not the Tn antigen on IgA1, whereas VVA bound well to IgA1 (FIG. 2G). The binding of both Remab6 and ReBaGs6 towards native human IgA1 from 4 donors were tested by Western blot and no binding was observed (FIG. 2C). These results demonstrate that both Remab6 and ReBaGs6 bind glycoproteins expressing the Tn antigen, but are unable to bind to IgA1 glycoforms expressing the Tn antigen.

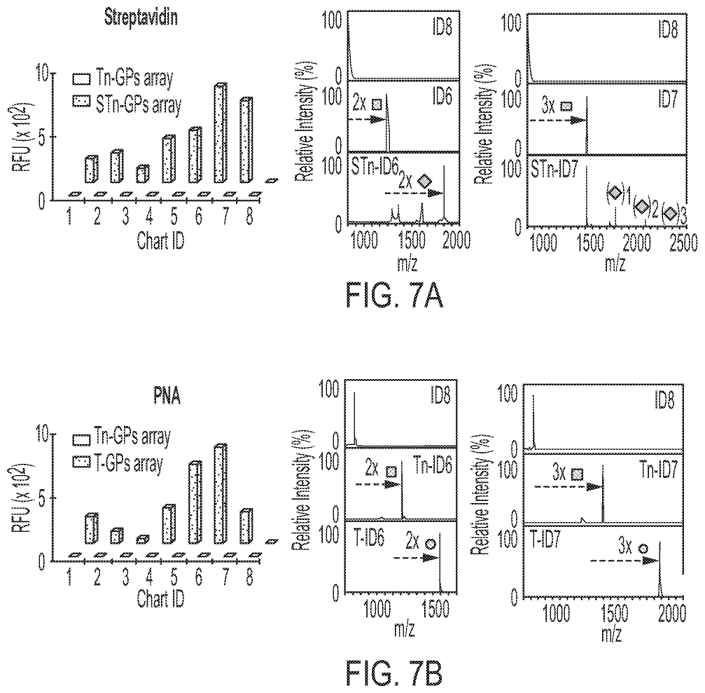

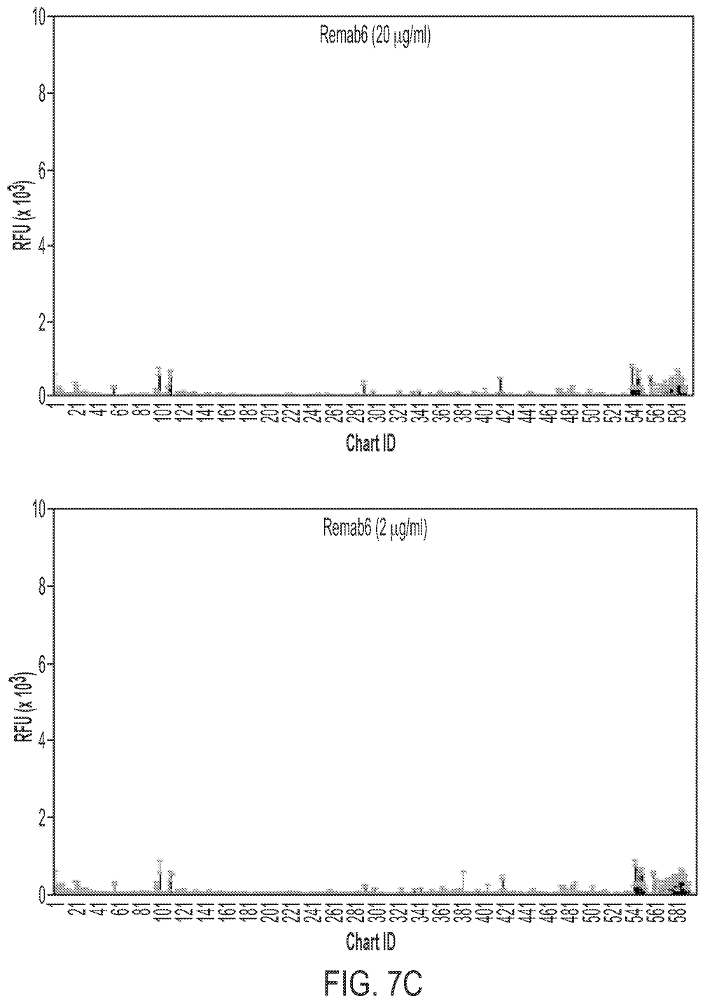

Interactions of Remab6 with Tn derivatives were also identified, e.g. STn (Neu5Aca2-6GalNAcα1-Ser/Thr) and T (Galβ1-3GalNAcα1-Ser/Thr) antigens, by enzymatically synthesizing them directly on the Tn glycopeptide array (FIG. 7A-7B). Partial sialylation of Tn antigen with recombinant ST6GalNAc-I to generate STn, significantly decreased binding of Remab6, with the signal remaining above background only for the MUC2 glycopeptide containing three consecutive Tn sites (ID7) (FIG. 2D). In the microarray platform, it is possible that the three adjacent Tn sites could not be efficiently sialylated, compared to the same peptide sequence with two adjacent Tn sites (ID6), and therefore the binding signal observed after sialylation was due to the incomplete Tn sialylation reaction. To test this hypothesis, the reaction product after sialylation in solution by MALDI-TOF MS analysis was characterized, and it was found that the triple-Tn-MUC2 (ID7) substrate was much less efficiently sialylated than the double-Tn-MUC2 (ID6) substrate (FIG. 7A). In fact, a strong signal from the non-sialylated peptide was present after the reaction. A T glycopeptide array was enzymatically synthesized by incubating the Tn glycopeptide array slides with recombinant T-synthase, thus modifying the Tn antigen by addition of β1,3-linked galactose. This modification inhibited Remab6 binding (FIG. 2E, FIG. 7B). Furthermore, both Remab6 and ReBaGs6 binding to Asialo-BSM were inhibited by addition of soluble GalNAc, but not GlcNAc. More importantly, ReBaGs6 exhibited a relatively strong binding to glycoproteins with the Tn antigen (K_{d}=5.43×10⁻⁸ M), while Remab6 as a chimeric human IgG1 showed a slightly lower binding affinity (K_{d}=8.29×10⁻⁷ M). The binding constant of ReBaGs6 is close to that of VVA (FIG. 2F). Finally, the CFG mammalian glycan microarray results show that Remab6 does not bind to BGA, Tn-related glycans or other glycans expressing terminal α-GalNAc (FIG. 7C). These results demonstrate that Remab6 is highly specific to the Tn antigen, especially in the clustered Tn-glycopeptide form, but does not detectably interact with STn antigen, T antigen, or with Tn antigen on human IgA1.

### Remab6 Robustness Inflow Cytometry and Immunofluorescence Applications

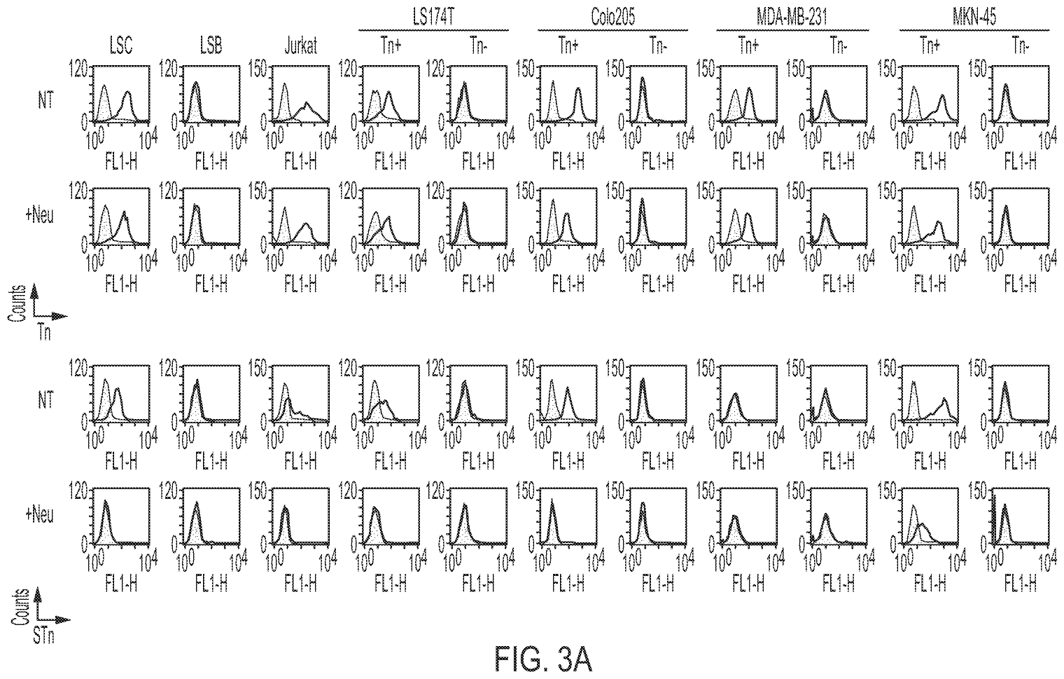

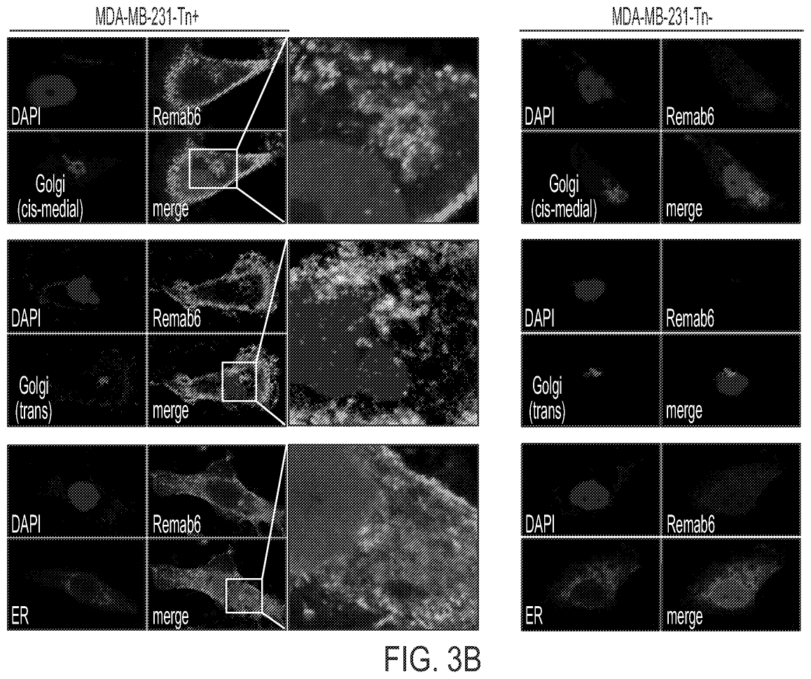

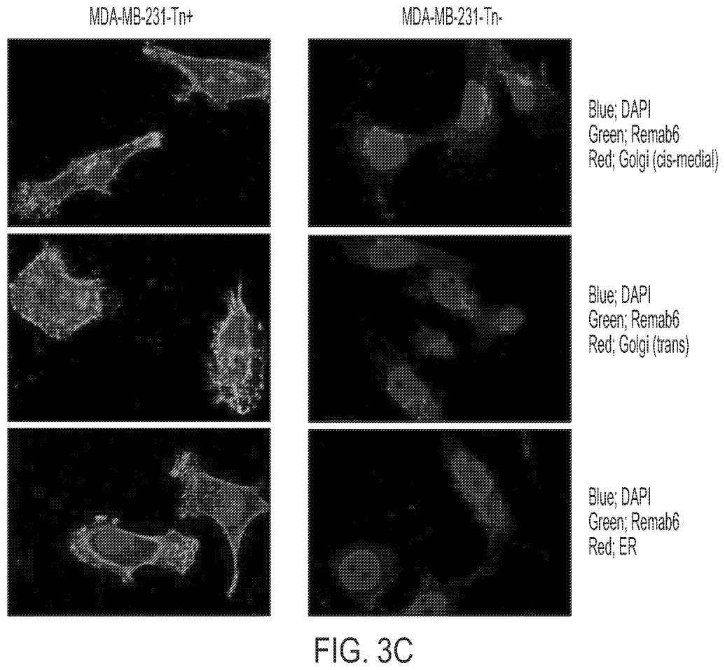

Like BaGs6, Remab6 stained a variety of Tn-positive Simple Cell lines by flow cytometry, but not their Tn-negative counterparts for LSC/LSB, LS174T, Colo205, MDA-MB-231, MKN-45, and Jurkat cell lines (FIG. 3A). Confocal microscopy analysis with Remab6 showed similar selectivity to Tn-positive cells in MDA-MB-231 cell line. Staining was observed on the cell surface and intracellular compartments, which was less co-localized in cis-medial Golgi than trans Golgi apparatus, but not co-localized in ER (FIGS. 3B-3C). These data demonstrate that Remab6 is a specific reagent for a variety of biochemical assays and can be used to distinguish intracellular versus extracellular Tn antigen presentation.

### Immunohistochemical Staining in IEC-Cosmc KO Mice and Human Cancer Cell Block Sections and Human Cancer Tissues Array

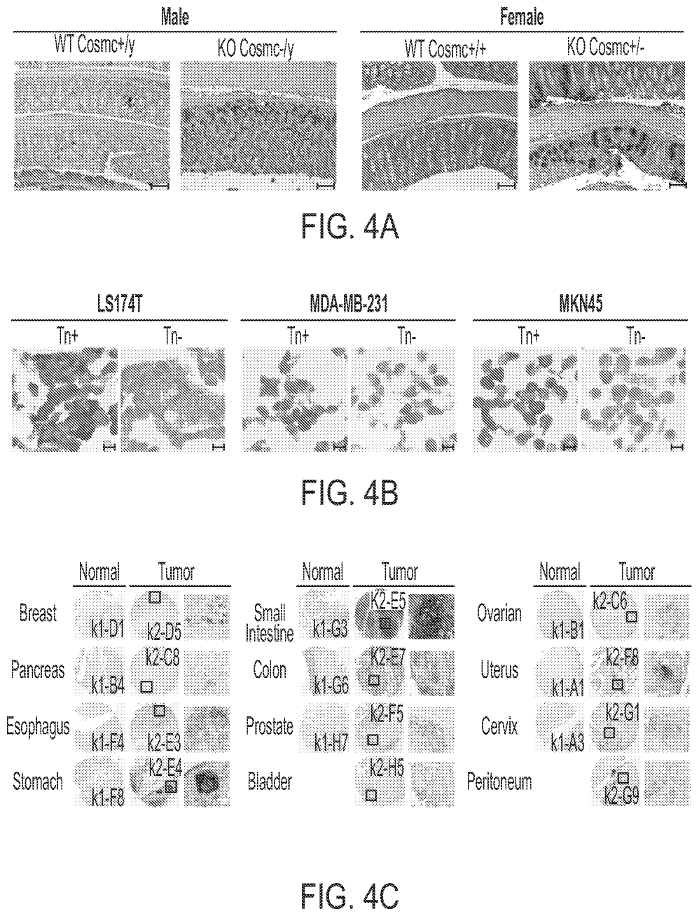

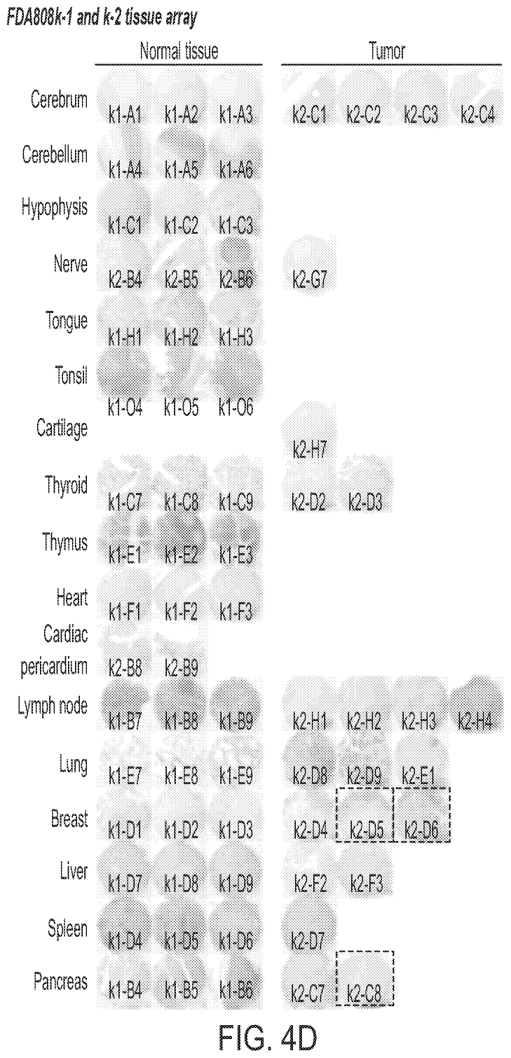

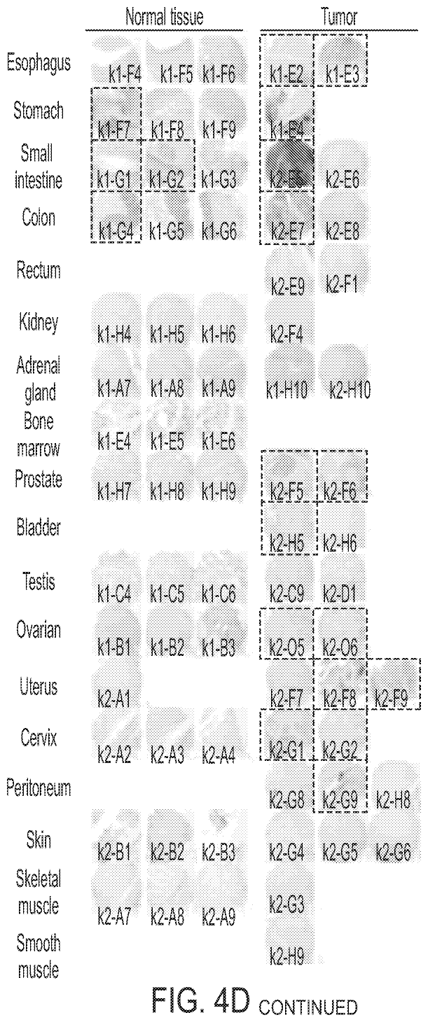

To further define the specificity of Remab6 using mammalian tissues, and whether Remab6 is specific to tissues expressing the Tn antigen on glycoproteins, prior development of the IEC-Cosmc KO mice was exploited. Male KO abundantly express the Tn and STn truncated O-glycans in colorectum and small intestine, and female mosaics exhibit ˜50% expression (28). Immunohistochemical staining with Remab6 showed highly positive staining in Cosmc KO tissues, but not normal tissues (FIG. 4A). Immunohistochemical staining in IEC-Cosmc KO mice with Remab6-HRP and Remab6-Fab-HRP also showed focused, strong Tn staining in small intestine-colon-rectum sections in villi-specific Cosmc KO mice compared to WT (FIG. 4D).

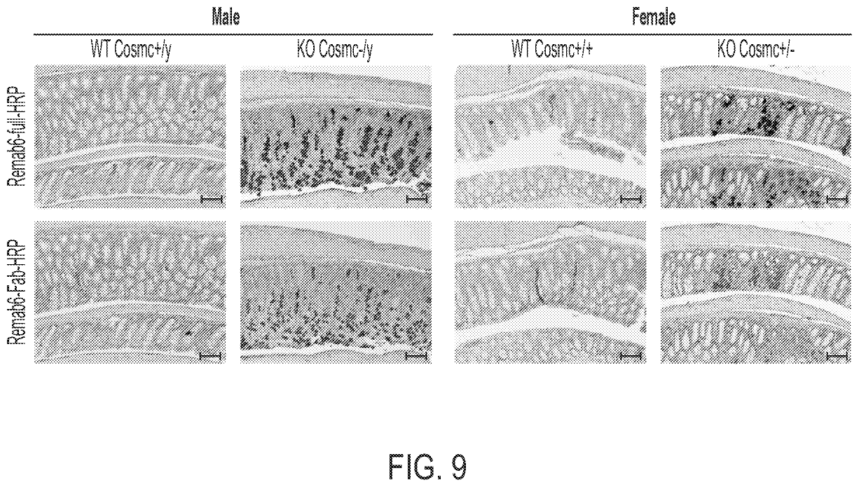

Immunohistochemical staining using human cancer cell block sections including human colorectal, breast and gastric cancer cell lines were assessed, in which Remab6 specifically stained those previously identified as being potentially Tn-positive (FIG. 4B). Finally, Remab6 was screened on a large set of normal and malignant samples on tissue arrays. The results demonstrate that Remab6 stains breast, gastrointestinal, prostate, ovarian, cervical, and pancreatic tissues of tumor origin, but rarely stained normal tissues (FIG. 4C, FIG. 9, Tables 2.1, 2.2). In tabulating the Tn staining data using the cancer tissue array, 27 tumor tissue types were tested and 12 showed positive staining (44% of tumor tissue types tested), compared to the 30 normal tissue types tested where only 3 showed positive staining (10% of normal tissue types) (Table 3). Interestingly, however, in these normal tissues, when positive, staining was only observed intracellularly, not on the cell surface, whereas malignant tissue stained both intra- and extracellularly. Intracellular staining of normal tissue represents immature glycoproteins in transit during biosynthesis where the Tn antigen is an intermediate. In addition, the extent and intensity of Tn expression was significantly greater in malignant tissues compared to normal tissue from the same organ, noting that these arrays contain multiple cell types in both normal and tumor tissues. These data demonstrate that Remab6 is specific for the Tn antigen in tumor cell surfaces and thus may be useful for detecting carcinomas.

### Identification of Tn Containing Glycoproteins in Colorectal Cancer Cell Line by LC/ESI-MS/MS

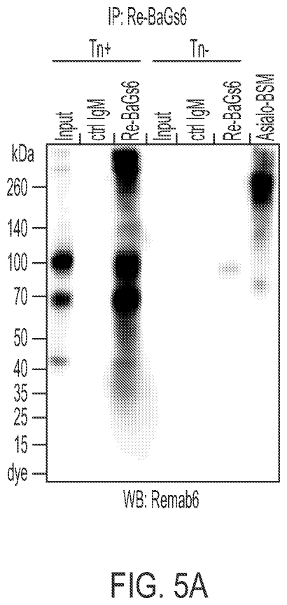

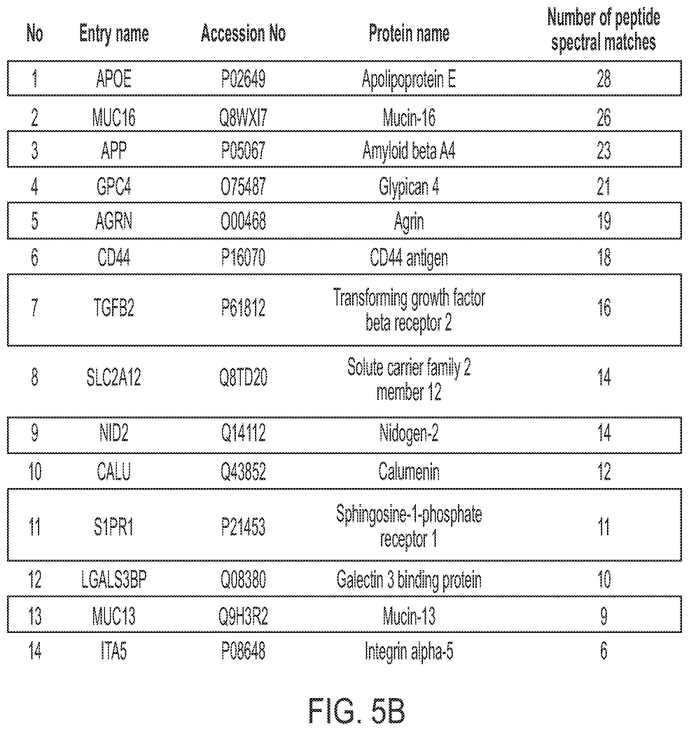

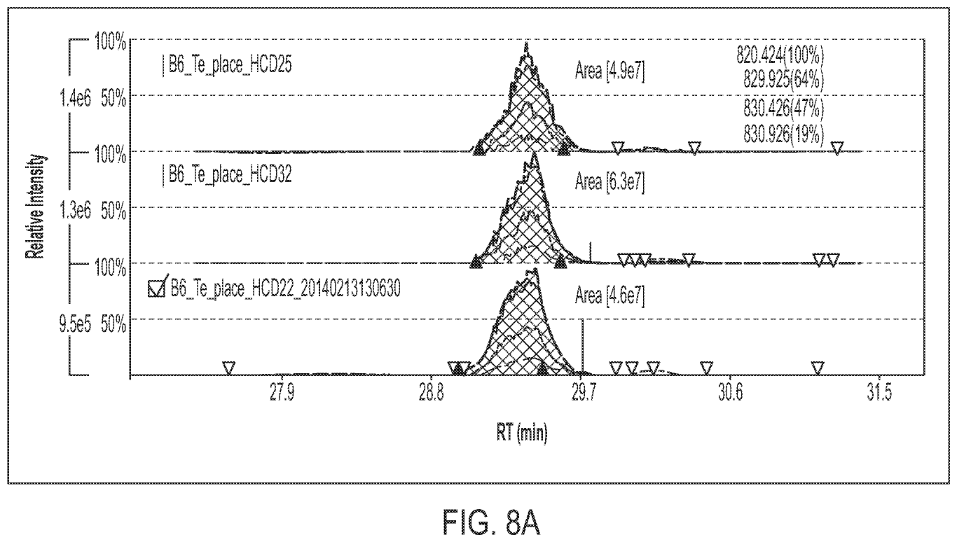

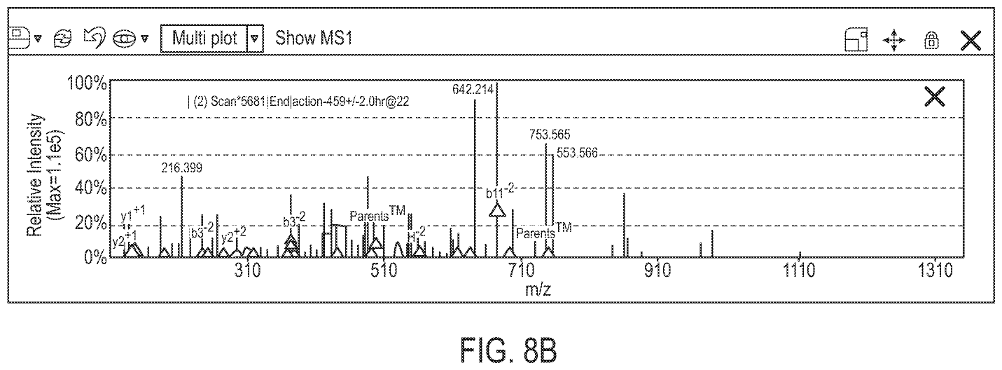

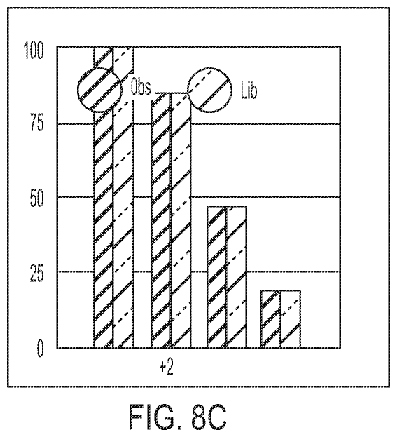

ReBaGs6 was sought to be used to identify Tn-containing glycoproteins that might be useful in analyses for biomarkers, as well as to explore potential mechanisms by which Tn antigen may be involved in tumor progression. To this end, the glycoproteins recognized by ReBaGs6 in Colo205 Simple Cells (Tn-positive) engineered by deletion of the Cosmc gene from parental Colo205 cells was examined. ReBaGs6 was conjugated to UltraLink beads and the conjugate was used for immunoprecipitation experiments using Colo205 Simple Cell (Tn-positive) lysates. A number of Tn-containing glycoproteins were captured, as identified by subsequent Western blot using Remab6 (FIG. 5A). As a control, glycopeptide sequences from Asialo-BSM was examined. Several Tn-containing peptide sequences were identified from Asialo-BSM under multiple higher collision dissociation (HCD) energies (FIGS. 8A-8C). The monoisotopic precursor isotopic pattern was matched against theoretical peptide for several peptide spectral matches identified via decoy database search with SEQUEST.

With success in these control studies on Asialo-BSM, similar instrument settings to identify tryptic glycopeptides using immunoprecipitates in Colo205 Simple Cells (Tn-positive) were used. Using this approach, several unique glycoproteins and unique peptide sequences with GalNAc modifications were identified from the Colo205 Simple Cell line (Tn-positive), but not the control Tn-negative Colo205 cell line (FIG. 5B). GO annotation of the identified glycopeptides is specific to either membrane glycoproteins or their secreted counterparts, and a number of such tryptic glycopeptides containing GalNAc are listed (Table 4). Interestingly, It was also observed GalNAc on tyrosine residues in several glycopeptides. This modification was observed in a recent study (35), but there have been few reports of its presence. These Tn-containing glycoproteins also included several sites predicted to be O-glycosylated, but not yet proven by chemical analysis, such as LC-MS/MS or western blot analysis. Together, the results indicate that both Remab6 and ReBaGs6 do not require specific amino acid backbones for recognition, and that these antibodies specifically recognize the Tn antigen on several unique glycoproteins in Colo205 cells. These unique glycoproteins will be useful in future biomarker studies.

(iii) Discussion

Provided herein is a set of recombinant anti-Tn monoclonal antibodies, human IgG1 Remab6 and murine IgM ReBaGs6. These antibodies are specific for the Tn antigen in glycoproteins and do not simply recognize terminal α-linked GalNAc residues, as may occur in BGA and other types of glycans. Importantly, the engineered antibodies do not recognize the Tn antigen present in the hinge region of IgA1. Furthermore, normal murine tissues did not bind to Remab6, whereas in murine tissues from mice engineered to lack Cosmc and express the Tn antigen, extensive staining was observed. The recombinant anti-Tn antibodies also allowed identification of novel Tn-positive glycoproteins in a human tumor cell line.

### Anti-Glycan Antibodies

Alteration of glycan structures in cancer is a common feature and has led to the development of glycoprotein-based biomarkers, including glycan- or glycoprotein-targeted antibodies, such as CA19-9, CA15-3, CA125, and CA72-4 (36). Several antibodies against those targets have been developed, but not all glycan structures are equally immunogenic, biasing the production of antibodies. Glycan determinants recognized by antibodies and other glycan binding proteins (GBPs) minimally contain 2-6 monosaccharides, limiting the generation of mAbs against single monosaccharides, such as GalNAc (37). From the traditional immunology viewpoint, glycans alone elicit T cell-independent immunity, whereas glycopeptides elicit glycopeptide-MHC-II interactions for presentation and T-cell dependency (38). Interestingly, while some Tn-containing MUC1 glycopeptides elicit T cell-dependent immune response, other glycopeptides with identical aglycon backbones but distinct Tn attachment sites do not, despite being endocytosed by DCs, suggesting that the selection of glycosylation sites is a critical key for immunogen design (39).

This complexity in the immune response has prompted many laboratories to generate monoclonal antibodies against the Tn antigen by immunizing with whole cancer cells (40-45), membrane proteins/cultured supernatants from cancer cells (32,40,42,46-50), Tn-linker molecules and synthesized Tn glycopeptides as a immunogen (33,51-57), and phage display (58,59). Some of these anti-Tn antibodies have been demonstrated to inhibit the growth of colon and breast cancer cell lines (60), tumor rejection (61), anti-tumor activity in vitro and in vivo (44,50,59,62,63), and used for in vivo imaging (54,64). However, monoclonal antibodies to the Tn antigen are notably difficult to generate, expensive to produce, and their specificities are often not well characterized, especially in regard to whether the anti-Tn antibodies recognize the Tn-positive IgA1 glycoform (18). This latter feature is generally not tested nor appreciated. Therefore, the anti-Tn antibodies that do not recognize IgA1 provide a valuable alternative that can be reproducibly made on a large scale and retain the fine specificity that was characterized herein. Previous study has demonstrated that Tn-modified glycopeptides show drastic conformational changes, which might give rise to unpredicted immunogenic epitopes (25). As both Remab6 and ReBaGs6 do not show cross reactivity to Tn antigen on IgA1, and can recognize tumor cell-expressed glycoproteins, the results suggest that these antibodies could be promising therapeutics and diagnostics in cancer.

### Remab6 is Specific for the Tn Antigen on Multiple Unique Glycoproteins in Cancer

Remab6 was functional in multiple types of assays, and provided clues to understand the mechanisms by which Tn-containing glycoproteins could be involved in cancer progression by LC/ESI-MS/MS analysis with immunoprecipitation (FIGS. 5A-5B). Consistent with prior studies, some of the Tn-containing glycoproteins have been seen previously using VVA lectin chromatography with a Cosmc knock out cell line and LC-MSn analysis (12, 65), and the established GlycoDomainViewer database (66). Several glycoproteins, such as APOE, MUC16, AGRN, CD44, and ITA5, that were identified using VVA lectin chromatography (35), were also identified. In this study, however, several additional and novel Tn-containing glycoproteins (FIG. 5B, Table 4) were identified. Among the novel proteins in the list, the unique glycopeptides in APP (Amyloid beta A4) were first identified in the present study, although APP was identified as an O-glycosylated glycoprotein in a prior study (67). Among the novel O-glycosylated glycoproteins identified include TGFβ2, SLC2A12, NID2, CALU, S1PR1, and MUC13, all of which have suggested roles in colorectal cancer.

For example, TGFβ2 is associated with the hypoxic tumor microenvironment to promote cancer cell stemness and chemoresistance in colorectal carcinoma (68). SLC2A12 (GLUT12) is correlated with the androgen receptor (AR) in multiple clinical cohorts, and is required for maximal androgen-mediated glucose uptake to promote cell growth in prostate cancer (69). NID2 is characterized as a tumor suppressor gene, and serves to maintain the extracellular matrix environment, to suppress liver metastasis by regulating EGFR/Akt and Integrin/FAK/PLCc pathways (70). CALU is an extracellular molecule, and stabilizes fibulin-1, and regulates cell migration via the ERK1/2 signaling pathway in hepatocellular and pancreatic carcinomas (71). S1PR1 is an important factor in promoting metastasis via the tumor microenvironment in multiple carcinomas (72). MUC13 is known to play a protective role in colorectal cancer by activating the NF-κB pathway, whereas another well-known mucin MUC16 plays an opposite role in extending tumor metastasis (73).

Of note, tyrosine O-GalNAcylation has been reported for a handful of glycopeptides in a recent study (35). In addition to the more well-known PTM identifications on tyrosine such as phosphorylation and sulfation, tyrosine glycosylation is typically not considered for data analysis as commonly as serine and threonine, should be considered for all future MS analyses. This tyrosine GalNAcylation could be orchestrated with serine or threonine GalNAcylation to affect oncogenic properties but that remains to be studied.

Overall, the results demonstrate that Remab6 does not recognize any particular amino acid backbone, yet the peptide expression is required for antigen expression and some peptide presentations may not be recognized by Remab6. The inhibition data indicate that Asialo-BSM is a better inhibitor of BaGs6, compared to VVA, and the monosaccharide GalNAc only weakly inhibits BaGs6. This suggests that there is little cross reactivity with other glycan structures terminating in α-linked GalNAc, in contrast to VVA and HPA, which simply recognize terminal α-linked GalNAc-R residues. This is in line with the requirement for multiple clustered sites (FIG. 2B), and as shown in previous binding studies with BaGs6 (25), and the sequence requirements suggest the peptide backbone plays a role, or is an important factor for spatially orienting the sugar residues. To date, several groups have demonstrated that Tn-containing glycoproteins modulate cell proliferation, invasion, metastatic potential, and immunosurveillance in some types of cancers (74-87). The surface expression of Tn-positive glycoproteins in tumor cells suggest that Remab6 could exhibit anti-tumor efficacy.

### Remab6 is Useful for a Diagnostic Biomarker in Several Types of Cancer, as Well as a Promising Targeted Therapy

Remab6 has potential as a diagnostic marker to detect Tn-carrying mucins in the serum or feces of cancer patients. The results with a human cancer tissue array indicate that Remab6 could be useful for the detection of Tn-expressing human carcinomas using biopsies with patients or CT/MRI imaging with limited off-target effects, since there was little reactivity on the surface of normal tissues (FIG. 4C and FIG. 9). The antibodies described here can be used in several therapeutic applications, including: 1) antibody-drug conjugates (ADC) technique, 2) CAR-T cell engineering, 3) ADCC/CDC-based immunotherapy, and 4) bispecific antibody (bsAb) technology. Recently, ADC with Chi-Tn, a mouse/human chimeric antibody for Tn antigen, showed anti-tumor efficacy when the antibody was internalized, and the cytotoxic drug is active in early and recycling endosomes (63). MUC1-Tn engineering CAR-T cells have been established and showed a potent antitumor efficacy in xenograft models of T cell leukemia and pancreatic cancer (88). These data indicate that Remab6 is a promising tool for detection, diagnosis, and therapeutic treatment in human carcinomas.

TABLE 1
specificity of Remab6 and ReBaGs6
on a Tn glycopeptide microarray

| Chart | | | SEQ |
|---|---|---|---|
| ID | Detail | Sequence | ID NO: |
| 1 | A-MUC2 | Ac-PT*TTPLK-NH2 | 79 |
| 2 | B-MUC2 | Ac-PTT*TPLK-NH2 | 79 |
| 3 | C-MUC2 | Ac-PTTT*PLK-NH2 | 79 |
| 4 | D-MUC2 | Ac-PT*T*TPLK-NH2 | 79 |
| 5 | E-MUC2 | Ac-PT*TT*PLK-NH2 | 79 |
| 6 | F-MUC2 | Ac-PTT*T*PLK-NH2 | 79 |
| 7 | G-MUC2 | Ac-PT*T*T*PLK-NH2 | 79 |
| 8 | R-MUC2 | Ac-PTTTPLK-NH2 | 79 |
| 9 | a-Dystroglycan | Ac-PPTTTTKKP-NH2 | 77 |
| 10 | MUC5AC | H2N-GTTPSPVPT*TSTTSAP-OH | 69 |
| 11 | EA2 | Ac-PTTDSTT*PAPTTK-NH2 | 78 |
| 12 | EA2-R | Ac-PTTDSTTPAPTTK-NH2 | 78 |
| 13 | a-Dystroglycan | Ac-PPT*T*T*T*KKP-HN2 | 77 |
| 14 | MUC1-1 | H2N-TSAPDT*RDAP-NH2 | 80 |
| 15 | MUC1-1R | H2N-TSAPDTRDAP-NH2 | 80 |
| 16 | MUC1-2 | H2N-APGS*T*APP-NH2 | 68 |
| 17 | MUC1-2R | H2N-APGSTAPP-NH2 | 68 |
| 18 | PADRE Tn3b | H2N-GaKcVAAWTLKAAaT*T*T*G-CONH2 | 81 |
| 19 | Tn3 linker | Ac-T*T*T*-NH(CH2)3NH2 | |
| 20 | Tn linker | Ac-T*-NH(CH2)3NH2 | |
| 21 | Peptide-4 | H2N-KTTT-CONH2 | 75 |
| 22 | Peptide-5 | H2N-KTTTG-CONH2 | 76 |
| 23 | Ser-GalNAc1 | H2N-Ser(a-D-GalNAc)-NH2 | |
| 24 | Ser-GalNAc2 | H2N-Ser(a-D-GalNAc)-OH | |
| 25 | Thr-GalNAc1 | H2N-Thr(a-D-GalNAc)-NH2 | |
| 26 | Thr-GalNAc2 | H2N-Thr(a-D-GalNAc)-OH | |
| 27 | IgA-Pep01 | H2N-KPVPST*PPT*PS*C-OH | 73 |
| 28 | IgA-Pep02 | H2N-KPVPSTPPTPSC-OH | 73 |
| 29 | IgA-Pep03 | H2N-KPVPS*TPPTPSC-OH | 73 |
| 30 | IgA-Pep04 | H2N-KPST*PPT*PS*PS*C-OH | 71 |
| 31 | IgA-Pep05 | H2N-KPSTPPTPSPSC-OH | 71 |
| 32 | IgA-Pep06 | H2N-KT*PPT*PS*PS*TPC-OH | 74 |
| 33 | IgA-Pep07 | H2N-KTPPTPSPSTPC-OH | 74 |
| 34 | IgA-Pep08 | H2N-KTPPTPSPST*PC-OH | 74 |
| 35 | IgA-Pep09 | H2N-KPT*PS*PS*TPPT*C-OH | 72 |
| 36 | IgA-Pep10 | H2N-KPSPSTPPTPSC-OH | 70 |
| 37 | IgA-Pep11 | H2N-KPS*PS*TPPT*PSC-OH | 70 |
| 38 | IgA-Pep12 | H2N-KPSTPPTPSPSC-OH | 71 |
| 39 | IgA-Pep13 | H2N-KPS*TPPT*PSPSC-OH | 71 |
| 40 | IgA-Pep14 | H2N-KPSTPPTPSPSC-OH | 71 |
| 41 | IgA-Pep15 | H2N-KPST*PPTPS*PS*C-OH | 71 |
| 42 | IgA-Pep16 | H2N-KPSTPPTPS*PSC-OH | 71 |
| 43 | IgA-Pep17 | H2N-KPSTPPTPSPS*C-OH | 71 |
| 44 | IgA-Pep18 | H2N-KPST*PPTPSPSC-OH | 71 |
| 45 | Phosphate Buffer | | |

*GalNAc on Serine, Threonine, or Tyrosine

TABLE 2.1
FDA808k-1 Multiple organ normal tissue array

| | | | | | | | | | c | |
|---|---|---|---|---|---|---|---|---|---|---|
| FDA808k-1 | | | Organ/ | Pathology | a | | | b | Tn | |
| No | Age | Sex | Anatomic Site | diagnosis | TNM | Grade | Stage | Type | expression | |
| A1 | 49 | M | Cerebrum | Cerebrum | — | — | — | Normal | — | |
| | | | | tissue | | | | | | |
| A2 | 38 | F | Cerebrum | Cerebrum | — | — | — | Normal | — | |
| | | | | tissue | | | | | | |
| A3 | 50 | F | Cerebrum | Cerebrum | — | — | — | Normal | — | |
| | | | | tissue | | | | | | |
| A4 | 24 | F | Cerebellum | Cerebellum | — | — | — | Normal | — | |
| | | | | tissue | | | | | | |
| A5 | 32 | M | Cerebellum | Cerebellum | — | — | — | Normal | — | |
| | | | | tissue | | | | | | |
| A6 | 45 | M | Cerebellum | Cerebellum | — | — | — | Normal | — | |
| | | | | tissue | | | | | | |
| A7 | 45 | M | Adrenal | Adrenal gland | — | — | — | Normal | — | |
| | | | gland | tissue | | | | | | |
| A8 | 27 | M | Adrenal | Adrenal gland | — | — | — | Normal | — | |
| | | | gland | tissue | | | | | | |
| A9 | 43 | M | Adrenal | Adrenal gland | — | — | — | Normal | — | |
| | | | gland | tissue | | | | | | |
| B1 | 45 | F | Ovary | Adjacent | — | — | — | NAT | — | |
| | | | | normal ovary | | | | | | |
| | | | | tissue | | | | | | |
| B2 | 35 | F | Ovary | Adjacent | — | — | — | NAT | — | |
| | | | | normal ovary | | | | | | |
| | | | | tissue | | | | | | |
| B3 | 64 | F | Ovary | Adjacent | — | — | — | NAT | — | |
| | | | | normal ovary | | | | | | |
| | | | | tissue | | | | | | |
| B4 | 40 | F | Pancreas | Pancreas tissue | — | — | — | Normal | — | |
| B5 | 21 | F | Pancreas | Pancreas tissue | — | — | — | Normal | — | |
| B6 | 27 | M | Pancreas | Pancreas tissue | — | — | — | Normal | — | |
| B7 | 41 | F | Lymph | Lymph node | — | — | — | Normal | — | |
| | | | node | tissue | | | | | | |
| B8 | 43 | M | Lymph | Lymph node | — | — | — | Normal | — | |
| | | | node | tissue | | | | | | |
| B9 | 35 | M | Lymph | Lymph node | — | — | — | Normal | — | |
| | | | node | tissue | | | | | | |
| C1 | — | — | Hypophysis | Hypophysis | — | — | — | Normal | — | |
| | | | | tissue | | | | | | |
| C2 | 34 | M | Hypophysis | Hypophysis | — | — | — | Normal | — | |
| | | | | tissue | | | | | | |
| C3 | 21 | F | Hypophysis | Hypophysis | — | — | — | Normal | — | |
| | | | | tissue | | | | | | |
| C4 | 35 | M | Testis | Testis tissue | — | — | — | Normal | — | |
| C5 | 65 | M | Testis | Adjacent | — | — | — | NAT | — | |
| | | | | normal testis | | | | | | |
| | | | | tissue | | | | | | |
| C6 | 74 | M | Testis | Adjacent | — | — | — | NAT | — | |
| | | | | normal testis | | | | | | |
| | | | | tissue | | | | | | |
| C7 | 22 | M | Thyroid | Thyroid gland | — | — | — | Normal | — | |
| | | | gland | tissue | | | | | | |
| C8 | 40 | F | Thyroid | Thyroid gland | — | — | — | Normal | — | |
| | | | gland | tissue | | | | | | |
| C9 | 35 | M | Thyroid | Thyroid gland | — | — | — | Normal | — | |
| | | | gland | tissue | | | | | | |
| D1 | 29 | F | Breast | Cancer | — | — | — | AT | — | |
| | | | | adjacent breast | | | | | | |
| | | | | tissue | | | | | | |
| D2 | 21 | F | Breast | Breast tissue | — | — | — | Normal | — | |
| D3 | 35 | F | Breast | Adjacent | — | — | — | NAT | — | |
| | | | | normal breast | | | | | | |
| | | | | tissue | | | | | | |
| D4 | 38 | M | Spleen | Spleen tissue | — | — | — | Normal | — | |
| D5 | 21 | F | Spleen | Spleen tissue | — | — | — | Normal | — | |
| D6 | 23 | M | Spleen | Spleen tissue | — | — | — | Normal | — | |
| D7 | 34 | M | Tonsil | Tonsil tissue | — | — | — | Normal | — | |
| D8 | 21 | F | Tonsil | Tonsil tissue | — | — | — | Normal | — | |
| D9 | 45 | M | Tonsil | Tonsil tissue | — | — | — | Normal | — | |
| E1 | 16 | M | Thymus | Thymus gland | — | — | — | Normal | — | |
| | | | gland | tissue | | | | | | |
| E2 | 0.7 | M | Thymus | Adjacent | — | — | — | NAT | — | |
| | | | gland | normal thymus | | | | | | |
| | | | | gland tissue | | | | | | |
| E3 | 15 | F | Thymus | Thymus gland | — | — | — | Normal | — | |
| | | | gland | tissue | | | | | | |
| E4 | 21 | F | Bone | Bone marrow | — | — | — | Normal | — | |
| | | | marrow | tissue | | | | | | |
| E5 | 61 | F | Bone | Adjacent | — | — | — | NAT | — | |
| | | | marrow | normal bone | | | | | | |
| | | | | marrow tissue | | | | | | |
| E6 | 70 | M | Bone | Adjacent | — | — | — | NAT | — | |
| | | | marrow | normal bone | | | | | | |
| | | | | marrow tissue | | | | | | |
| E7 | 16 | F | Lung | Lung tissue | — | — | — | Normal | — | |
| E8 | 30 | M | Lung | Lung tissue | — | — | — | Normal | — | |
| E9 | 21 | F | Lung | Lung tissue | — | — | — | Normal | — | |
| F1 | 35 | F | Heart | Cardiac muscle | — | — | — | Normal | — | |
| | | | | tissue | | | | | | |
| F2 | 26 | M | Heart | Cardiac muscle | — | — | — | Normal | — | |
| | | | | tissue | | | | | | |
| F3 | 40 | M | Heart | Cardiac muscle | — | — | — | Normal | — | |
| | | | | tissue | | | | | | |
| F4 | 34 | M | Esophagus | Esophagus | — | — | — | Normal | — | |
| | | | | tissue | | | | | | |
| F5 | 30 | M | Esophagus | Esophagus | — | — | — | Normal | — | |
| | | | | tissue | | | | | | |
| F6 | 24 | M | Esophagus | Esophagus | — | — | — | Normal | — | |
| | | | | tissue | | | | | | |
| F7 | 24 | M | Stomach | Stomach tissue | — | — | — | Normal | Positive | * |
| F8 | 30 | M | Stomach | Stomach tissue | — | — | — | Normal | — | |
| F9 | 38 | M | Stomach | Stomach tissue | — | — | — | Normal | — | |
| G1 | 45 | M | Small | Small intestine | — | — | — | Normal | Positive | * |
| | | | intestine | tissue | | | | | | |
| G2 | 45 | M | Small | Small intestine | — | — | — | Normal | Positive | * |
| | | | intestine | tissue | | | | | | |
| G3 | 21 | F | Small | Small intestine | — | — | — | Normal | — | |
| | | | intestine | tissue | | | | | | |
| G4 | 30 | M | Colon | Colon tissue | — | — | — | Normal | Positive | * |
| G5 | 45 | M | Colon | Colon tissue | — | — | — | Normal | — | |
| G6 | 35 | M | Colon | Colon tissue | — | — | — | Normal | — | |
| G7 | 43 | M | Liver | Liver tissue | — | — | — | Normal | — | |
| G8 | 35 | M | Liver | Liver tissue | — | — | — | Normal | — | |
| G9 | 40 | M | Liver | Liver tissue | — | — | — | Normal | — | |
| H1 | 40 | M | Tongue | Salivary gland | — | — | — | Normal | — | |
| | | | | tissue | | | | | | |
| H2 | 35 | M | Tongue | Salivary gland | — | — | — | Normal | — | |
| | | | | tissue | | | | | | |
| H3 | 54 | F | Tongue | Adjacent | — | — | — | NAT | — | |
| | | | | normal | | | | | | |
| | | | | salivary gland | | | | | | |
| | | | | tissue | | | | | | |
| H4 | 38 | M | Kidney | Kidney tissue | — | — | — | Normal | — | |
| H5 | 47 | M | Kidney | Kidney tissue | — | — | — | Normal | — | |
| H6 | 50 | M | Kidney | Kidney tissue | — | — | — | Normal | — | |
| H7 | 43 | M | Prostate | Prostate tissue | — | — | — | Normal | — | |
| H8 | 31 | M | Prostate | Prostate tissue | — | — | — | Normal | — | |
| H9 | 35 | M | Prostate | Adjacent | — | — | — | NAT | — | |
| | | | | normal | | | | | | |
| | | | | prostate tissue | | | | | | |
| H10 | 42 | M | Adrenal | Pheochromocytoma | — | — | — | Malignant | — | |
| | | | gland | (tissue | | | | | | |
| | | | | marker) | | | | | | |

a. TNM grading
T—Primary tumor
Tx—Primary tumor cannot be assessed
T0—No evidence of primary tumor
Tis—Carcinoma in situ; intraepithelial or invasion of lamina propria
T1—Tumor invades submucosa
T2—Tumor invades muscularis propria
T3—Tumor invades through muscularis propria into subserosa or into non-peritonealized pericolic or perirectal tissues
T4—Tumor directly invades other organs or structures and/or perforate visceral peritoneum
N—Regional lymph nodes
Nx—Regional lymph nodes cannot be assessed
N0—No regional lymph node metastasis
N1—Metastasis in 1 to 3 regional lymph nodes
N2—Metastasis in 4 or more regional lymph nodes
M—Distant metastasis
Mx—Distant metastasis cannot be assessed
M0—No distant metastasis
M1—Distant metastasis
N—Regional lymph nodes
Nx—Regional lymph nodes cannot be assessed
N0—No regional lymph node metastasis
N1—Metastasis in 1 to 3 regional lymph nodes
N2—Metastasis in 4 or more regional lymph nodes
M—Distant metastasis
Mx—Distant metastasis cannot be assessed
M0—No distant metastasis
M1—Distant metastasis
b. Type
Normal—Normal tissue
NAT—Normal adjacent tissue
AT—Adjacent tissue
Malignant—Malignant tissue
c. Tn expression
Indicates positive or negative staining in IHC staining
* Intracellular staining

TABLE 2.2
FDA808k-1 Multiple organ normal tissue array

| | | | | | | | | | c |
|---|---|---|---|---|---|---|---|---|---|
| FDA808k-2 | | | Organ/ | Pathology | a | | | b | Tn |
| No | Age | Sex | Anatomic Site | diagnosis | TNM | Grade | Stage | Type | expression |
| A1 | 48 | F | Uterus | Adjacent | — | — | — | NAT | — |
| | | | | normal | | | | | |
| | | | | endometrium | | | | | |
| | | | | tissue | | | | | |
| A2 | 18 | F | Uterus | Endometrium | — | — | — | Normal | — |
| | | | | tissue | | | | | |
| A3 | 41 | F | Cervix | Adjacent | — | — | — | NAT | — |
| | | | | normal | | | | | |
| | | | | endometrium | | | | | |
| | | | | tissue | | | | | |
| A4 | 36 | F | Cervix | Cancer | — | — | — | AT | — |
| | | | | adjacent | | | | | |
| | | | | cervical canals | | | | | |
| | | | | tissue | | | | | |
| A5 | 72 | F | Cervix | Adjacent | — | — | — | NAT | — |
| | | | | normal cervix | | | | | |
| | | | | tissue | | | | | |
| A6 | 65 | F | Cervix | Adjacent | — | — | — | NAT | — |
| | | | | normal | | | | | |
| | | | | cervical canals | | | | | |
| | | | | tissue with | | | | | |
| | | | | squamous | | | | | |
| | | | | metaplasia | | | | | |
| A7 | 40 | M | Skeletal | Skeletal | — | — | — | Normal | — |
| | | | muscle | muscle tissue | | | | | |
| A8 | 49 | F | Skeletal | Adjacent | — | — | — | NAT | — |
| | | | muscle | normal | | | | | |
| | | | | skeletal muscle | | | | | |
| | | | | tissue | | | | | |
| A9 | 50 | M | Skeletal | Skeletal | — | — | — | Normal | — |
| | | | muscle | muscle tissue | | | | | |
| B1 | 34 | M | Skin | Skin tissue of | — | — | — | Normal | — |
| | | | | chest part | | | | | |
| B2 | 21 | M | Skin | Skin tissue | — | — | — | Normal | — |
| B3 | 35 | M | Skin | Skin tissue of | — | — | — | Normal | — |
| | | | | abdomen part | | | | | |
| B4 | 23 | M | Nerve | Peripheral | — | — | — | Normal | — |
| | | | | nerve tissue | | | | | |
| B5 | 25 | M | Nerve | Peripheral | — | — | — | Normal | — |
| | | | | nerve tissue | | | | | |
| B6 | 50 | M | Nerve | Peripheral | — | — | — | Normal | — |
| | | | | nerve tissue | | | | | |
| B7 | 33 | M | Lung | Diaphragm | — | — | — | Normal | — |
| | | | | tissue and | | | | | |
| | | | | mesothelial | | | | | |
| | | | | tissue | | | | | |
| B8 | 19 | M | Cardiac | Pericardium | — | — | — | Normal | — |
| | | | pericardium | tissue and | | | | | |
| | | | | mesothelium | | | | | |
| | | | | tissue | | | | | |
| B9 | 43 | M | Cardiac | Diaphragm | — | — | — | Normal | — |
| | | | pericardium | tissue and | | | | | |
| | | | | mesothelial | | | | | |
| | | | | tissue | | | | | |
| C1 | 17 | M | Cerebrum | Glioblastoma | — | 4 | — | Malignant | — |
| C2 | 65 | F | Cerebrum | Atypical | — | — | — | Malignant | — |
| | | | | meningioma | | | | | |
| C3 | 40 | F | Cerebrum | Glioblastoma | — | 4 | — | Malignant | — |
| C4 | 39 | F | Cerebrum | Oligodendroglioma | — | 2 | — | Malignant | — |
| C5 | 62 | F | Ovary | Endometrioid | T2N0M0 | 3 | II | Malignant | Positive |
| | | | | carcinoma | | | | | |
| C6 | 29 | F | Ovary | Mucinous | T3N0M0 | 3 | III | Malignant | Positive |
| | | | | adenocarcinoma | | | | | |
| C7 | 16 | F | Pancreas | Islet cell tumor | — | — | — | Malignant | — |
| C8 | 64 | M | Pancreas | Adenocarcinoma | T3N0M0 | 3 | IIA | Malignant | Positive |
| C9 | 32 | M | Testis | Seminoma | T1N0M0 | — | IA | Malignant | — |
| D1 | 30 | M | Testis | Embryonal | T2N0M0 | — | IB | Malignant | — |
| | | | | carcinoma | | | | | |
| D2 | 33 | F | Thyroid | Medullary | T3N0M0 | — | II | Malignant | — |
| | | | gland | carcinoma | | | | | |
| D3 | 46 | F | Thyroid | Papillary | T4N0M0 | — | IVA | Malignant | — |
| | | | gland | carcinoma | | | | | |
| D4 | 67 | F | Breast | Intraductal | TisN0M0 | — | 0 | Malignant | — |
| | | | | carcinoma | | | | | |
| D5 | 58 | F | Breast | Invasive ductal | T3N1M0 | 2 | IIIA | Malignant | Positive |
| | | | | carcinoma | | | | | |
| D6 | 42 | F | Breast | Invasive ductal | T2N1M0 | 3 | IIB | Malignant | Positive |
| | | | | carcinoma | | | | | |
| D7 | 21 | M | Spleen | Diffuse B-cell | — | — | — | Malignant | — |
| | | | | lymphoma | | | | | |
| D8 | 61 | M | Lung | Small cell | T2N0M0 | — | IB | Malignant | — |
| | | | | undifferentiated | | | | | |
| | | | | carcinoma | | | | | |
| D9 | 64 | M | Lung | Squamous cell | T2N0M0 | 3 | IB | Malignant | — |
| | | | | carcinoma | | | | | |
| E1 | 42 | M | Lung | Adenocarcinoma | T2N0M0 | 3 | IB | Malignant | — |
| E2 | 58 | M | Esophagus | Squamous cell | T3N1M0 | 2 | IIIA | Malignant | Positive |
| | | | | carcinoma | | | | | |
| E3 | 63 | M | Esophagus | Adenocarcinoma | T3N0M0 | 3 | IIA | Malignant | Positive |
| E4 | 73 | F | Stomach | Adenocarcinoma | T2N1M0 | 3 | IIA | Malignant | Positive |
| E5 | 64 | F | Small | Adenocarcinoma | T4N0M0 | 3 | IIB | Malignant | Positive |
| | | | intestine | | | | | | |
| E6 | 71 | F | Small | Malignant | T2N0M0 | — | I | Malignant | — |
| | | | intestine | mesenchymoma | | | | | |
| E7 | 72 | F | Colon | Adenocarcinoma | T4N0M0 | 2 | IVA | Malignant | Positive |
| E8 | 54 | M | Colon | Mesenchymoma | T2N0M0 | — | I | Malignant | — |
| E9 | 36 | M | Rectum | Adenocarcinoma | T2N0M0 | 2 | I | Malignant | — |
| F1 | 63 | F | Rectum | Malignant | T2N0M0 | — | I | Malignant | — |
| | | | | mesenchymoma | | | | | |
| F2 | 55 | F | Liver | Hepatocellular | T3N0M0 | 2 | IIA | Malignant | — |
| | | | | carcinoma | | | | | |
| F3 | 17 | F | Liver | Hepatoblastoma | — | — | — | Malignant | — |
| F4 | 60 | M | Kidney | Clear cell | T2N0M0 | 1 | II | Malignant | — |
| | | | | carcinoma | | | | | |
| F5 | 80 | M | Prostate | Adenocarcinoma | T3N0M0 | 3 | III | Malignant | Positive |
| | | | | (Gleason | | | | | |
| | | | | grade: 4) | | | | | |
| F6 | 77 | M | Prostate | Adenocarcinoma | T2N0M0 | 3 | IIB | Malignant | Positive |
| | | | | (Gleason | | | | | |
| | | | | grade: 5) | | | | | |
| F7 | 56 | F | Uterus | Lowly | T2N0M0 | — | II | Malignant | — |
| | | | | malignant | | | | | |
| | | | | leiomyosarcoma | | | | | |
| F8 | 50 | F | Uterus | Endometrioid | T1bN0M0 | 1 | IB | Malignant | Positive |
| | | | | adenocarcinoma | | | | | |
| F9 | 57 | F | Uterus | Clear cell | T1bN0M0 | — | IB | Malignant | Positive |
| | | | | carcinoma | | | | | |
| G1 | 36 | F | Cervix | Squamous cell | T1bN0M0 | 3 | IB | Malignant | Positive |
| | | | | carcinoma | | | | | |
| G2 | 49 | F | Cervix | Squamous cell | T2N0M0 | 3 | II | Malignant | Positive |
| | | | | carcinoma | | | | | |
| G3 | 20 | F | Skeletal | Embryonal | T1aN0M0 | — | IA | Malignant | — |
| | | | muscle | rhabdomyosarcom | | | | | |
| | | | | of left leg | | | | | |
| G4 | 70 | F | Rectum | Malignant | — | — | — | Malignant | — |
| | | | | melanoma | | | | | |
| G5 | 85 | F | Skin | Basal cell | T2N0M0 | — | II | Malignant | — |
| | | | | carcinoma of | | | | | |
| | | | | left face | | | | | |
| G6 | 64 | M | Skin | Squamous cell | T3N0M0 | 2 | III | Malignant | — |
| | | | | carcinoma of | | | | | |
| | | | | chest wall | | | | | |
| G7 | 21 | M | Nerve | Neurofibroma | — | — | — | Malignant | — |
| | | | | of back | | | | | |
| G8 | 1 | F | Retroperitoneum | Neuroblastoma | — | — | — | Malignant | — |
| G9 | 60 | M | Peritoneum | Malignant | T2N0M0 | — | II | Malignant | Positive |
| | | | | mesothelioma | | | | | |
| H1 | 55 | M | Lymph node | Diffuse B cell | — | — | — | Malignant | — |
| | | | | lymphoma of | | | | | |
| | | | | armpit | | | | | |
| H2 | 50 | F | Lymph node | Diffuse B cell | — | — | — | Malignant | — |
| | | | | lymphoma of | | | | | |
| | | | | right thigh | | | | | |
| H3 | 27 | M | Lymph node | Mixed cell | — | — | — | Malignant | — |
| | | | | type Hodgkin's | | | | | |
| | | | | lymphoma of | | | | | |
| | | | | left groin | | | | | |
| H4 | 36 | M | Lymph node | Anaplastic | — | — | — | Malignant | — |
| | | | | large cell | | | | | |
| | | | | lymphoma of | | | | | |
| | | | | left groin | | | | | |
| H5 | 58 | F | Bladder | High grade | T2N0M0 | — | II | Malignant | Positive |
| | | | | urothelial | | | | | |
| | | | | carcinoma | | | | | |
| H6 | 62 | M | Bladder | Leiomyosarcoma | T2N0M0 | — | II | Malignant | — |
| H7 | 37 | F | Cartilage | Osteosarcoma | T2N0M0 | — | IIB | Malignant | — |
| | | | | of left femur | | | | | |
| | | | | lower section | | | | | |
| H8 | 48 | F | Retroperitoneum | Highly | T2N0M0 | — | IIB | Malignant | — |
| | | | | malignant | | | | | |
| | | | | pleomorphic | | | | | |
| | | | | rhabdomysarcoma | | | | | |
| H9 | 60 | F | Smooth | Moderate | T2N0M0 | — | IIB | Malignant | — |
| | | | muscle | malignant | | | | | |
| | | | | leiomyosarcoma | | | | | |
| | | | | of left | | | | | |
| | | | | buttock | | | | | |
| H10 | 42 | M | Adrenal | Pheochromocytoma | — | — | — | Malignant | — |
| | | | gland | (tissue | | | | | |
| | | | | marker) | | | | | |

a. TNM grading
T—Primary tumor
Tx—Primary tumor cannot be assessed
T0—No evidence of primary tumor
Tis—Carcinoma in situ; intraepithelial or invasion of lamina propria
T1—Tumor invades submucosa
T2—Tumor invades muscularis propria
T3—Tumor invades through muscularis propria into subserosa or into non-peritonealized pericolic or perirectal tissues
T4—Tumor directly invades other organs or structures and/or perforate visceral peritoneum
N—Regional lymph nodes
Nx—Regional lymph nodes cannot be assessed
N0—No regional lymph node metastasis
N1—Metastasis in 1 to 3 regional lymph nodes
N2—Metastasis in 4 or more regional lymph nodes
M—Distant metastasis
Mx—Distant metastasis cannot be assessed
M0—No distant metastasis
M1—Distant metastasis
N—Regional lymph nodes
Nx—Regional lymph nodes cannot be assessed
N0—No regional lymph node metastasis
N1—Metastasis in 1 to 3 regional lymph nodes
N2—Metastasis in 4 or more regional lymph nodes
M—Distant metastasis
Mx—Distant metastasis cannot be assessed
M0—No distant metastasis
M1—Distant metastasis
b. Type
Normal—Normal tissue
NAT—Normal adjacent tissue
AT—Adjacent tissue
Malignant—Malignant tissue
c. Tn expression
Indicates positive or negative staining in IHC staining
* Intracellular staining

TABLE 3
Tn Staining in human cancer tissue array

| Tissue | Malignant | Normal |
|---|---|---|
| Cerebrum | 0/4 | 0/3 |
| Cerebellum | — | 0/3 |
| Hypophysis | — | 0/3 |
| Nerve | 0/1 | 0/3 |
| Tongue | — | 0/3 |
| Tonsil | — | 0/3 |
| Cartilage | 0/1 | — |
| Thyroid | 0/2 | 0/3 |
| Thymus | — | 0/3 |
| Heart | — | 0/3 |
| Cardiac pericardium | — | 0/2 |
| Lymph node | 0/4 | 0/3 |
| Lung | 0/3 | 0/3 |
| Breast | 2/3 | 0/3 |
| Liver | 0/2 | 0/3 |
| Spleen | 0/1 | 0/3 |
| Pancreas | 1/2 | 0/3 |
| Esophagus | 2/2 | 0/3 |
| Stomach | 1/1 | 1/3 * |
| Small intestine | 1/2 | 2/3 * |
| Colon | 1/2 | 1/3 * |
| Rectum | 0/2 | — |
| Kidney | 0/1 | 0/3 |
| Adrenal gland | 0/2 | 0/3 |
| Bone marrow | — | 0/3 |
| Prostate | 2/2 | 0/3 |
| Bladder | 1/2 | — |
| Testis | 0/2 | 0/3 |
| Ovarian | 2/2 | 0/3 |
| Uterus | 2/3 | 0/1 |
| Cervix | 2/2 | 0/3 |
| Peritoneum | 1/3 | — |
| Skin | 0/3 | 0/3 |
| Skeletal muscle | 0/1 | 0/3 |
| Smooth muscle | 0/1 | — |

* Intracellular staining

TABLE 4
Identification of peptide sequences by LC/ESI-MS/MS analysis

| | | Peptide/ | Peptide/ | Peptide/ |
|---|---|---|---|---|
| | Entry | Glycopeptide | Glycopeptide | Glycopeptide |
| No | name | Sequence #1 | Sequence #2 | Sequence #3 |
| 1 | APOE | ALMDET*MKELK | LRARMEEMGS*RT*R | S*WFEPLVEDMQRQW |
| | | (SEQ ID NO: 18) | (SEQ ID NO: 30) | AGLVEK |
| | | | | (SEQ ID NO: 42) |
| 2 | MUC16 | TEALSLGRT*ST*PG | DT*FNDSAAPQST*T | ISTSAPLSSS*AS*V |
| | | PAQSTIS | WPETSPR | LDNK |
| | | (SEQ ID NO: 19) | (SEQ ID NO: 31) | (SEQ ID NO: 43) |
| 3 | APP | LALENY*IT*ALQAV | FLHQERMDVCETHLH | PAADRGLTTRª |
| | | PPRPR | WHT*VAKETCSEK | (SEQ ID NO: 44) |
| | | (SEQ ID NO: 20) | (SEQ ID NO: 32) | |
| 4 | AGRN | TTAAPTTRRPPT*T* | TTAAPTTRRª | KDFRSVRª |
| | | APSRVPGR | (SEQ ID NO: 33) | (SEQ ID NO: 45) |
| | | (SEQ ID NO: 21) | | |
| 5 | CD44 | KPS*GLNGEAS*KS* | SQEMVHLVNKª | RMDMDSSHSITLQPT |
| | | QEMVHLVNK | (SEQ ID NO: 34) | *ANPNTGLVEDLDR |
| | | (SEQ ID NO: 22) | | (SEQ ID NO: 46) |
| 6 | TGFB2 | LSSTWET*GK | EHPCVES*MK | IFPY*EEY*ASWKTE |
| | | (SEQ ID NO: 23) | (SEQ ID NO: 35) | K |
| | | | | (SEQ ID NO: 47) |
| 7 | SLC2A12 | SS*LM**PLR | GCS*LEQISMELAKV | Y*MFGLVIPLGVLQA |
| | | (SEQ ID NO: 24) | NY*VKNNICFMSHHQ | IAMYFLPPS*PRFLV |
| | | | EELVPK | MKGQEGAASK |
| | | | (SEQ ID NO: 36) | (SEQ ID NO: 48) |
| 8 | NID2 | KALEGLQY*PFAVT* | IEVAKLDGT*QR | IESALLDGSERª |
| | | SYGK | (SEQ ID NO: 37) | (SEQ ID NO: 49) |
| | | (SEQ ID NO: 25) | | |
| 9 | CALU | MDLRQFLMCLSLCTA | IDGDKDGFVT*VDEL | TFDQLTPEESKERª |
| | | FALS*KPT*EK | KDWIK | (SEQ ID NO: 50) |
| | | (SEQ ID NO: 26) | (SEQ ID NO: 38) | |
| 10 | S1PR1 | YIT*MLK | IMS*CCK | T*CDILFR |
| | | (SEQ ID NO: 27) | (SEQ ID NO: 39) | (SEQ ID NO: 51) |
| 11 | MUC13 | HS*MAYQDLHSEITS | CAFGY*S*GLDCKDK | STGFTNLGAEGSVFP |
| | | *LFKDVF | (SEQ ID NO: 40) | K^{a} |
| | | (SEQ ID NO: 28) | | (SEQ ID NO: 52) |
| 12 | ITA5 | ERQVAT*AVQWT*K | HPGNFSS*LS*CDY* | RSLPYGTAMEKª |
| | | (SEQ ID NO: 29) | FAVNQSR | (SEQ ID NO: 53) |
| | | | (SEQ ID NO: 41) | |

*GalNAc on Serine, Threonine, or Tyrosine (in red)
**Oxidation on Methionine
^{a}Not identified site-specific GalNAcylation

### Example 2: Generation of FX Gene Knockout HEK293 Freestyle (FXKO) Cell Line to Prepare Recombinant Antibodies Lacking Fucose in their N-Glycans

### (i) Materials and Methods

### Generation of FX Gene Knockout HEK293 Freestyle (FXKO) Cell Line

A cell line that is devoid of the sugar fucose in its glycans was sought to be generated. To this end the gene named TSTA3 was targeted. This gene encodes the enzyme GDP-L-fucose synthase, also termed the FX enzyme, which converts GDP-4-keto-6-deoxymannose to GDP-fucose. In the absence of the enzyme cells are unable to generate GDP-fucose, the required donor for glycan fucosylation. However, the cells will retain a ‘salvage’ pathway, whereby free L-fucose can be taken up by cells and directly converted to GDP-fucose, and thereby all fucosylation is dependent on fucose supplied in the culture media. Primers were prepared to target the FX gene with the sequence (TCACCATGGTCTCATCTATC (SEQ ID NO: 17); +chr8:143614783-805, Exon4), and these are termed sgRNA.

HEK293 freestyle cells (HEK293FS) were cotransfected with a sgRNA and Cas9 mRNA following manufacturer's instruction (Dharmacon). Transfected cells were sorted by their binding to a fucose-specific lectin, AAL, and cells lacking binding to AAL were collected and deemed the AAL-negative population. These cells were termed the FXKO cell line.

### Production of Afucosylated Remab6 (Remab6-FXKO)

Remab6 construct was transfected into FXKO cells, and cultured supernatant was purified with protein A column. N-glycan on afucosylated-Remab6 antibody (Remab6-FXKO) was analyzed by MALDI-TOF mass spectrometry.

### Uptake Endogenous L-Fucose and Refucosylation on Remab6-FXKO

Remab6-transfected FXKO cells were cultured with various concentration of L-fucose in the defined culture media for 4 days, and analyzed by flow cytometry with AAL staining. N-glycans present on the recombinant Remab6-FXKO antibody were analyzed by MALDI-MS mass spectrometry.

### Complement Dependent Cytotoxicity (CDC) and Antibody Dependent Cell Cytotoxicity (ADCC)

CDC assay was performed with Remab6-WT and Remab6-FXKO antibody (5 μg/ml) in presence of 20% human serum for 4 h at 37 C, and % of lysis was measured by annexin V and PI double positive population. ADCC assay was performed with Remab6-WT and Remab6-FXKO antibody (5 μg/ml) in presence of NK-92 cell line (ATCC) for 4 h at 37 C, and % of lysis was measured by LDH release.

### (ii) Results

FXKO cells were established in CRISPR/Cas-9 system as described in Materials and methods. Deficient fucosylated glycans on cell surface were observed by flowcytometry with AAL lectin, and N-glycan profile. The isolated recombinant Remab6 IgG1 antibody produced by the Remab6-FXKO cells was analyzed by MALDI-TOF mass spectrometry.

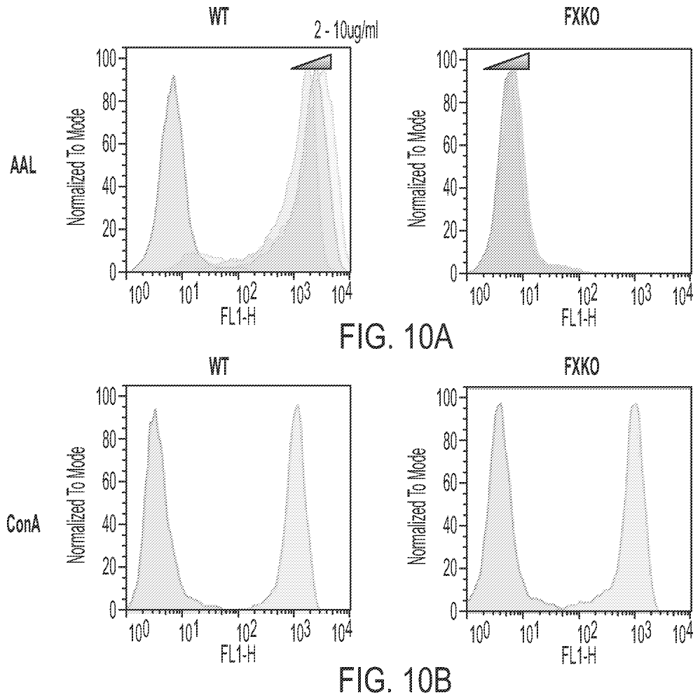

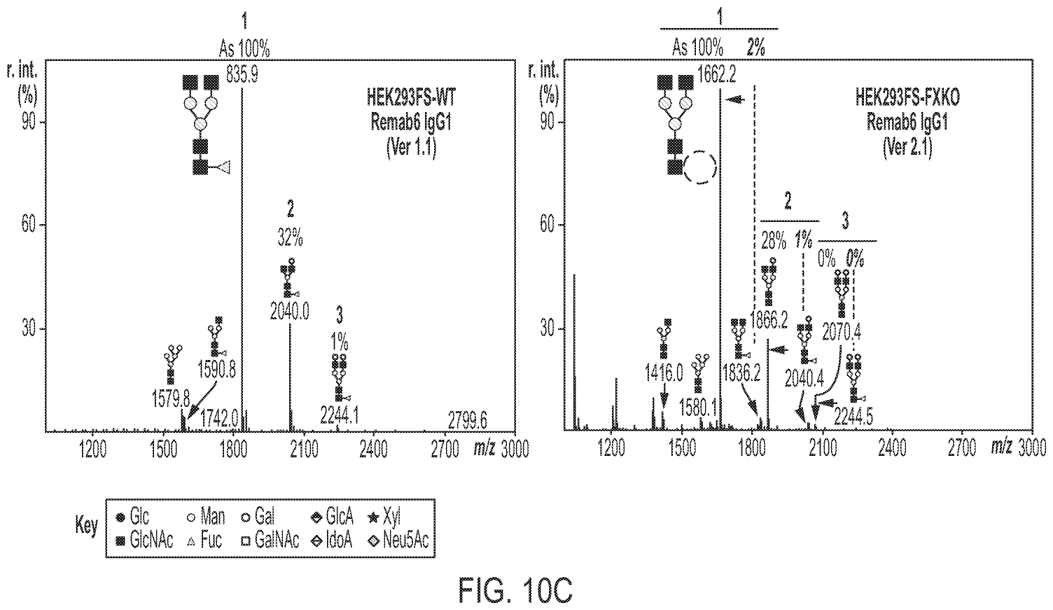

The results demonstrate that the FXKO cells expressing recombinant Remab6 IgG1 antibody do not stain appreciably with the lectin AAL that binds to fucose on cell surface glycoproteins, and that the antibody derived from FXKO cells expressing recombinant Remab6 IgG1 antibody is also lacking fucose, compared to control cells and antibody from the wild-type WT cells. Remab6-WT is 97% fucosylated, and Remab6-FXKO is 98% afucosylated (FIGS. 10A-10C).

The pathway for producing GDP-Fuc in cells is shown on the upper left, and the FX enzyme targeted for deletion is indicated in the figure. Through this pathway the ‘salvage’ pathway for generating GDP-Fuc from L-fucose is indicated. FXKO cells were cultured with L-fucose in defined media for 4 days (as shown on the upper right), and the degree of fucosylation was observed by flowcytometry with AAL lectin staining. N-glycans on the recombinant Remab6-FXKO antibody produced by the cells under such conditions were analyzed by MALDI-TOF mass spectrometry, shown in the bottom, and the degree of fucosylation ranged from 9.1% to 91% using 1 to 10 micromolar L-fucose, respectively.

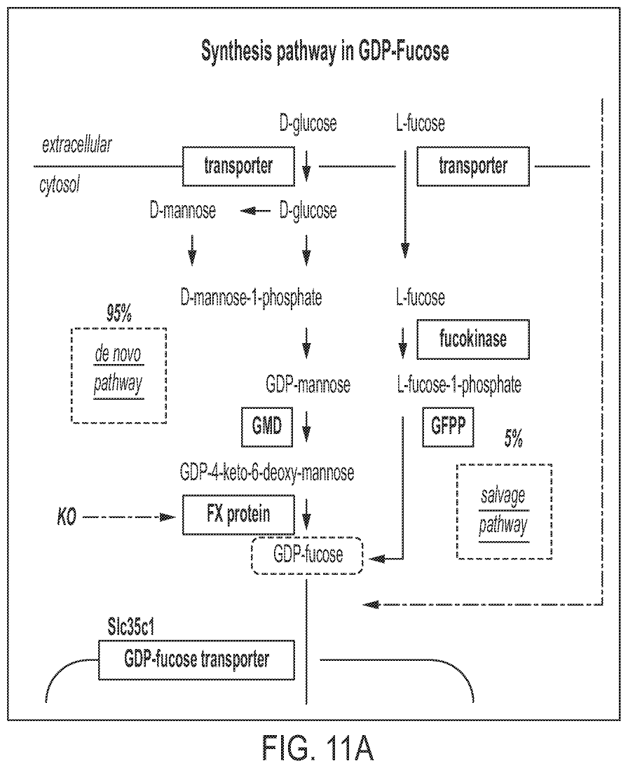

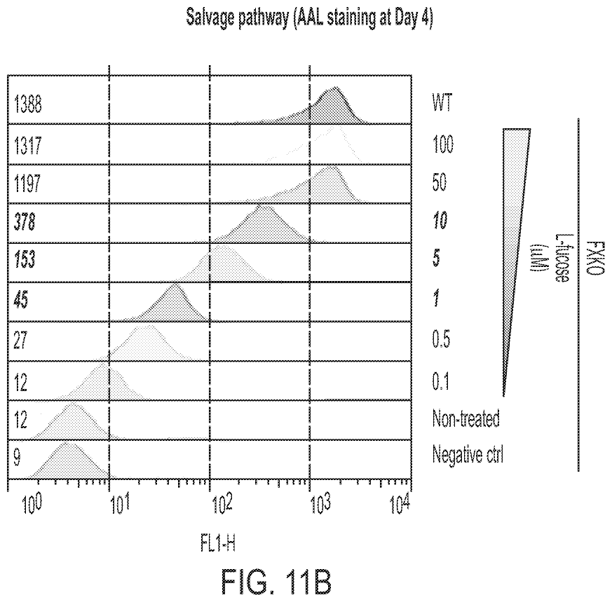

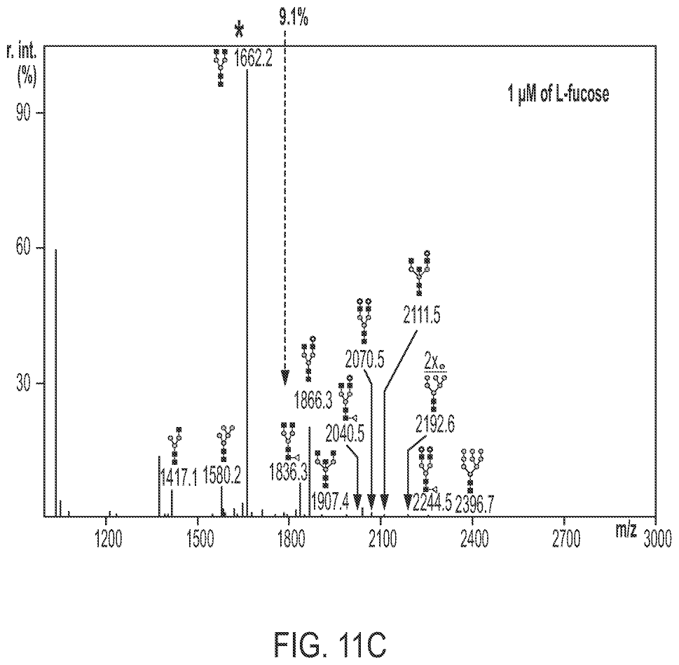

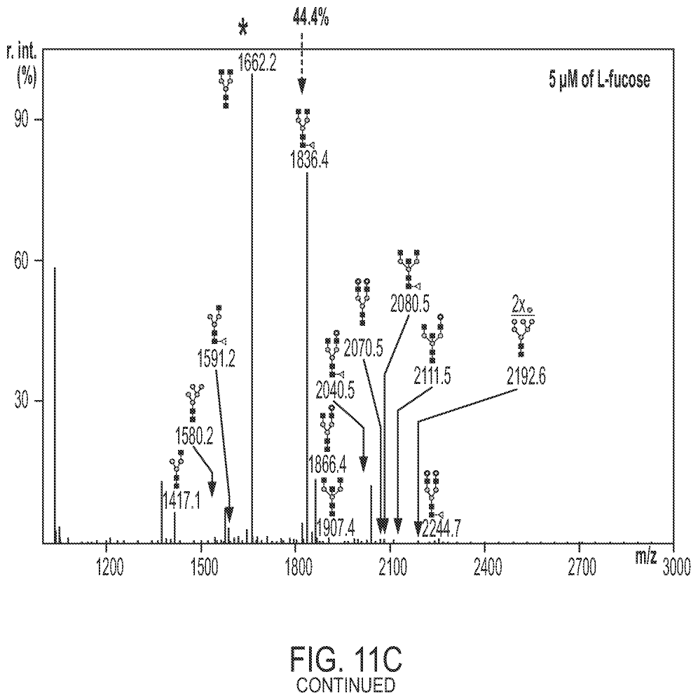

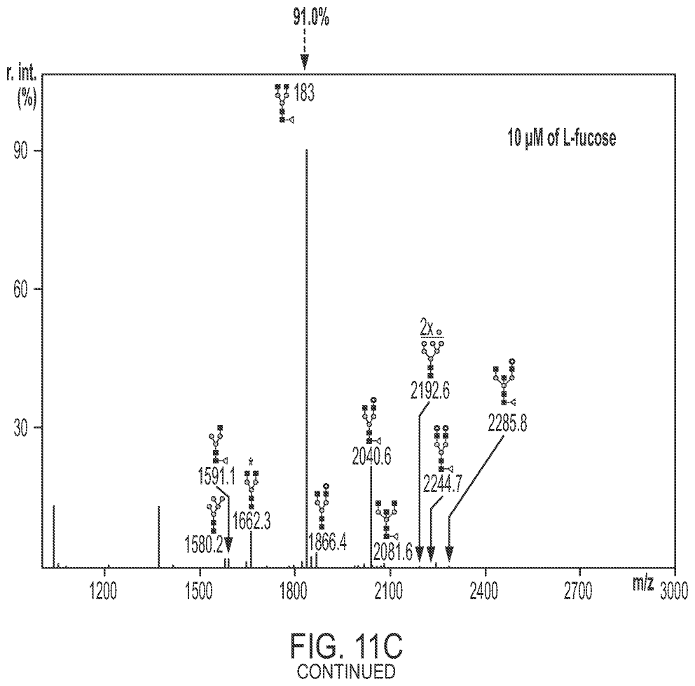

The results demonstrate that increasing the amount of fucose in the culture media from 0.1 micromolar to 100 micromolar causes increased staining of cells with AAL lectin, and that the addition of 10 micromolar fucose also causes high level of fucosylation of the recombinant remab6 antibody (FIGS. 11A-11C).

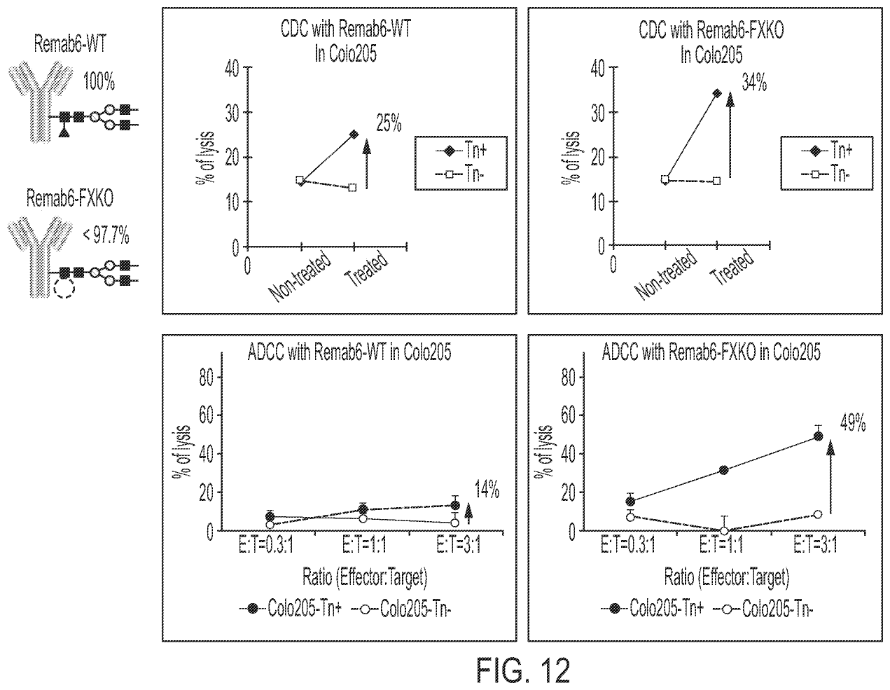

The complement-dependent cytotoxicity (CDC) assay was performed with Remab6-WT and Remab6-FXKO antibody in presence of human serum that naturally contains complement. The antibody-dependent cellular cytotoxicity (ADCC) activity assay was performed with Remab6-WT and Remab6-FXKO antibody in presence of NK cells, using Colo205 cells that are wild-type (WT) or Colo205 cells that express the target antigen known at Tn antigen recognized by the Remab6 antibody. A schematic of an IgG1 recombinant antibody with or without fucose is shown on the upper left (FIG. 12).

The results demonstrate that the afucosylated Remab6 antibody lacking fucose (termed Remab6-FXKO) is more activity in the ADCC activity than antibody containing fucose (termed Remab6-WT). However, in the CDC assays the two versions of the antibody with or without fucose are similarly activity toward the Colo205 cells that express the target antigen known at Tn antigen (FIG. 12).

### REFERENCES

1. Hakomori, S. (2001) Tumor-associated carbohydrate antigens defining tumor malignancy: basis for development of anti-cancer vaccines. Adv Exp Med Biol 491, 369-402
2. Fuster, M. M., and Esko, J. D. (2005) The sweet and sour of cancer: glycans as novel therapeutic targets. Nat Rev Cancer 5, 526-542
3. Ju, T., Wang, Y., Aryal, R. P., Lehoux, S. D., Ding, X., Kudelka, M. R., Cutler, C., Zeng, J., Wang, J., Sun, X., Heimburg-Molinaro, J., Smith, D. F., and Cummings, R. D. (2013) Tn and sialyl-Tn antigens, aberrant O-glycomics as human disease markers. Proteomics Clin Appl 7, 618-631
4. Ju, T., Aryal, R. P., Kudelka, M. R., Wang, Y., and Cummings, R. D. (2014) The Cosmc connection to the Tn antigen in cancer. Cancer Biomark 14, 63-81
5. Kudelka, M. R., Ju, T., Heimburg-Molinaro, J., and Cummings, R. D. (2015) Simple sugars to complex disease—mucin-type O-glycans in cancer. Adv Cancer Res 126, 53-135
6. Stowell, S. R., Ju, T., and Cummings, R. D. (2015) Protein glycosylation in cancer. Annu Rev Pathol 10, 473-510
7. Chia, J., Goh, G., and Bard, F. (2016) Short O-GalNAc glycans: regulation and role in tumor development and clinical perspectives. Biochim Biophys Acta 1860, 1623-1639
8. Bennett, E. P., Mandel, U., Clausen, H., Gerken, T. A., Fritz, T. A., and Tabak, L. A. (2012) Control of mucin-type O-glycosylation: a classification of the polypeptide GalNAc-transferase gene family. Glycobiology 22, 736-756
9. Ju, T., Brewer, K., D'Souza, A., Cummings, R. D., and Canfield, W. M. (2002) Cloning and expression of human core 1 beta1,3-galactosyltransferase. J Biol Chem 277, 178-186
10. Ju, T., and Cummings, R. D. (2002) A unique molecular chaperone Cosmc required for activity of the mammalian core 1 beta 3-galactosyltransferase. Proc Natl Acad Sci USA 99, 16613-16618
11. Ju, T., Lanneau, G. S., Gautam, T., Wang, Y., Xia, B., Stowell, S. R., Willard, M. T., Wang, W., Xia, J. Y., Zuna, R. E., Laszik, Z., Benbrook, D. M., Hanigan, M. H., and Cummings, R. D. (2008) Human tumor antigens Tn and sialyl Tn arise from mutations in Cosmc. Cancer Res 68, 1636-1646
12. Radhakrishnan, P., Dabelsteen, S., Madsen, F. B., Francavilla, C., Kopp, K. L., Steentoft, C., Vakhrushev, S. Y., Olsen, J. V., Hansen, L., Bennett, E. P., Woetmann, A., Yin, G., Chen, L., Song, H., Bak, M., Hlady, R. A., Peters, S. L., Opavsky, R., Thode, C., Qvortrup, K., Schjoldager, K. T., Clausen, H., Hollingsworth, M. A., and Wandall, H. H. (2014) Immature truncated O-glycophenotype of cancer directly induces oncogenic features. Proc Nat Acad Sci USA 111, E4066-4075
13. Jiang, Y., Liu, Z., Xu, F., Dong, X., Cheng, Y., Hu, Y., Gao, T., Liu, J., Yang, L., Jia, X., Qian, H., Wen, T., and An, G. (2018) Aberrant O-glycosylation contributes to tumorigenesis in human colorectal cancer. J Cell Mol Med 22, 4875-4885
14. Yuan, M. (1989) [The expression of Tn and S-Tn antigens in cancer and pre-malignant lesion of colorectal tissues by enzyme immunohistochemical method]. Zhonghua Bing Li Xue Za Zhi 18, 211-213
15. Itzkowitz, S. H., Bloom, E. J., Lau, T. S., and Kim, Y. S. (1992) Mucin associated Tn and sialosyl-Tn antigen expression in colorectal polyps. Gut 33, 518-523
16. Schumacher, U., Higgs, D., Loizidou, M., Pickering, R., Leathem, A., and Taylor, I. (1994) Helix pomatia agglutinin binding is a useful prognostic indicator in colorectal carcinoma. Cancer 74, 3104-3107
17. Mitchell, B. S., and Schumacher, U. (1999) The use of the lectin Helix pomatia agglutinin (HPA) as a prognostic indicator and as a tool in cancer research. Histol Histopathol 14, 217-226
18. Lehoux, S., Mi, R., Aryal, R. P., Wang, Y., Schjoldager, K. T., Clausen, H., van Die, I., Han, Y., Chapman, A. B., Cummings, R. D., and Ju, T. (2014) Identification of distinct glycoforms of IgA1 in plasma from patients with immunoglobulin A (IgA) nephropathy and healthy individuals. Mol Cell Proteomics 13, 3097-3113
19. Taylor-Papadimitriou, J., Burchell, J. M., Graham, R., and Beatson, R. (2018) Latest developments in MUC1 immunotherapy. Biochem Soc Trans 46, 659-668
20. Aithal, A., Rauth, S., Kshirsagar, P., Shah, A., Lakshmanan, I., Junker, W. M., Jain, M., Ponnusamy, M. P., and Batra, S. K. (2018) MUC16 as a novel target for cancer therapy. Expert Opin Ther Targets 22, 675-686
21. Springer, G. F., Desai, P. R., Wise, W., Carlstedt, S. C., Tegtmeyer, H., Stein, R., and Scanlon, E. F. (1990) Pancarcinoma T and Tn epitopes: autoimmunogens and diagnostic markers that reveal incipient carcinomas and help establish prognosis. Immunol Ser 53, 587-612
22. Brockhausen, I., Yang, J., Dickinson, N., Ogata, S., and Itzkowitz, S. H. (1998) Enzymatic basis for sialyl-Tn expression in human colon cancer cells. Glycoconj J 15, 595-603
23. Steentoft, C., Vakhrushev, S. Y., Joshi, H. J., Kong, Y., Vester-Christensen, M. B., Schjoldager, K. T., Lavrsen, K., Dabelsteen, S., Pedersen, N. B., Marcos-Silva, L., Gupta, R., Bennett, E. P., Mandel, U., Brunak, S., Wandall, H. H., Levery, S. B., and Clausen, H. (2013) Precision mapping of the human O-GalNAc glycoproteome through SimpleCell technology. EMBO J 32, 1478-1488
24. Ju, T., Cummings, R. D., and Canfield, W. M. (2002) Purification, characterization, and subunit structure of rat core 1 Beta1,3-galactosyltransferase. J Biol Chem 277, 169-177
25. Borgert, A., Heimburg-Molinaro, J., Song, X., Lasanajak, Y., Ju, T., Liu, M., Thompson, P., Ragupathi, G., Barany, G., Smith, D. F., Cummings, R. D., and Live, D. (2012) Deciphering structural elements of mucin glycoprotein recognition. ACS Chem Biol 7, 1031-1039
26. Heimburg-Molinaro, J., Song, X., Smith, D. F., and Cummings, R. D. (2011) Preparation and analysis of glycan microarrays. Curr Protoc Protein Sci Chapter 12, Unit12
27. Bern, M., Cai, Y., and Goldberg, D. (2007) Lookup peaks: a hybrid of de novo sequencing and database search for protein identification by tandem mass spectrometry. Anal Chem 79, 1393-1400
28. Kudelka, M. R., Hinrichs, B. H., Darby, T., Moreno, C. S., Nishio, H., Cutler, C. E., Wang, J., Wu, H., Zeng, J., Wang, Y., Ju, T., Stowell, S. R., Nusrat, A., Jones, R. M., Neish, A. S., and Cummings, R. D. (2016) Cosmc is an X-linked inflammatory bowel disease risk gene that spatially regulates gut microbiota and contributes to sex-specific risk. Proc Natl Acad Sci USA 113, 14787-14792
29. Tollefsen, S. E., and Kornfeld, R. (1983) The B4 lectin from Vicia villosa seeds interacts with N-acetylgalactosamine residues alpha-linked to serine or threonine residues in cell surface glycoproteins. J Biol Chem 258, 5172-5176
30. Tsuji T, O. T. (1986) Carbohydrate structures of bovine submaxillary mucin. Carbohydr Res 151, 391-402
31. Pancino, G. F., Osinaga, E., Vorauher, W., Kakouche, A., Mistro, D., Charpin, C., and Roseto, A. (1990) Production of a monoclonal antibody as immunohistochemical marker on paraffin embedded tissues using a new immunization method. Hybridoma 9, 389-395
32. Numata, Y., Nakada, H., Fukui, S., Kitagawa, H., Ozaki, K., Inoue, M., Kawasaki, T., Funakoshi, I., and Yamashina, I. (1990) A monoclonal antibody directed to Tn antigen. Biochem Biophys Res Commun 170, 981-985
33. Sorensen, A. L., Reis, C. A., Tarp, M. A., Mandel, U., Ramachandran, K., Sankaranarayanan, V., Schwientek, T., Graham, R., Taylor-Papadimitriou, J., Hollingsworth, M. A., Burchell, J., and Clausen, H. (2006) Chemoenzymatically synthesized multimeric Tn/STn MUC1 glycopeptides elicit cancer-specific anti-MUC1 antibody responses and override tolerance. Glycobiology 16, 96-107
34. Xu J L, D. M. (2000) Diversity in the CDR3 region of V(H) is sufficient for most antibody specificities. Immunity 13, 37-45
35. Vakhrushev, S. Y., Steentoft, C., Vester-Christensen, M. B., Bennett, E. P., Clausen, H., and Levery, S. B. (2013) Enhanced mass spectrometric mapping of the human GalNAc-type O-glycoproteome with SimpleCells. Mol Cell Proteomics 12, 932-944
36. Chen, Z. Q., Huang, L. S., and Zhu, B. (2018) Assessment of Seven Clinical Tumor Markers in Diagnosis of Non-Small-Cell Lung Cancer. Dis Markers 2018, 9845123
37. Cummings, R. D. (2009) The repertoire of glycan determinants in the human glycome. Mol Biosyst 5, 1087-1104
38. Singh, S. K., Streng-Ouwehand, I., Litjens, M., Kalay, H., Saeland, E., and van Kooyk, Y. (2011) Tumour-associated glycan modifications of antigen enhance MGL2 dependent uptake and MHC class I restricted CD8 T cell responses. Int J Cancer 128, 1371-1383
39. Vlad, A. M., Muller, S., Cudic, M., Paulsen, H., Otvos, L., Jr., Hanisch, F. G., and Finn, O. J. (2002) Complex carbohydrates are not removed during processing of glycoproteins by dendritic cells: processing of tumor antigen MUC1 glycopeptides for presentation to major histocompatibility complex class II-restricted T cells. J Exp Med 196, 1435-1446
40. Springer, G. F., Chandrasekaran, E. V., Desai, P. R., and Tegtmeyer, H. (1988) Blood group Tn-active macromolecules from human carcinomas and erythrocytes: characterization of and specific reactivity with mono- and poly-clonal anti-Tn antibodies induced by various immunogens. Carbohydr Res 178, 271-292
41. Bigbee, W. L., Langlois, R. G., Stanker, L. H., Vanderlaan, M., and Jensen, R. H. (1990) Flow cytometric analysis of erythrocyte populations in Tn syndrome blood using monoclonal antibodies to glycophorin A and the Tn antigen. Cytometry 11, 261-271
42. King, M. J., Parsons, S. F., Wu, A. M., and Jones, N. (1991) Immunochemical studies on the differential binding properties of two monoclonal antibodies reacting with Tn red cells. Transfusion 31, 142-149
43. Brooks, C. L., Schietinger, A., Borisova, S. N., Kufer, P., Okon, M., Hirama, T., Mackenzie, C. R., Wang, L. X., Schreiber, H., and Evans, S. V. (2010) Antibody recognition of a unique tumor-specific glycopeptide antigen. Proc Natl Acad Sci USA 107, 10056-10061
44. Welinder, C., Baldetorp, B., Borrebaeck, C., Fredlund, B. M., and Jansson, B. (2011) A new murine IgG1 anti-Tn monoclonal antibody with in vivo anti-tumor activity. Glycobiology 21, 1097-1107
45. Blixt, O., Lavrova, O. I., Mazurov, D. V., Clo, E., Kracun, S. K., Bovin, N. V., and Filatov, A. V. (2012) Analysis of Tn antigenicity with a panel of new IgM and IgG1 monoclonal antibodies raised against leukemic cells. Glycobiology 22, 529-542
46. Hirohashi, S., Clausen, H., Yamada, T., Shimosato, Y., and Hakomori, S. (1985) Blood group A cross-reacting epitope defined by monoclonal antibodies NCC-LU-35 and -81 expressed in cancer of blood group O or B individuals: its identification as Tn antigen. Proc Natl Acad Sci USA 82, 7039-7043
47. Takahashi, H. K., Metoki, R., and Hakomori, S. (1988) Immunoglobulin G3 monoclonal antibody directed to Tn antigen (tumor-associated alpha-N-acetylgalactosaminyl epitope) that does not cross-react with blood group A antigen. Cancer Res 48, 4361-4367
48. Huang, J., Byrd, J. C., Siddiki, B., Yuan, M., Lau, E., and Kim, Y. S. (1992) Monoclonal antibodies against partially deglycosylated colon cancer mucin that recognize Tn antigen. Dis Markers 10, 81-94
49. Osinaga, E., Bay, S., Tello, D., Babino, A., Pritsch, O., Assemat, K., Cantacuzene, D., Nakada, H., and Alzari, P. (2000) Analysis of the fine specificity of Tn-binding proteins using synthetic glycopeptide epitopes and a biosensor based on surface plasmon resonance spectroscopy. FEBS Lett 469, 24-28
50. Ando, H., Matsushita, T., Wakitani, M., Sato, T., Kodama-Nishida, S., Shibata, K., Shitara, K., and Ohta, S. (2008) Mouse-human chimeric anti-Tn IgG1 induced anti-tumor activity against Jurkat cells in vitro and in vivo. Biol Pharm Bull 31, 1739-1744
51. Longenecker, B. M., Willans, D. J., MacLean, G. D., Selvaraj, S., Suresh, M. R., and Noujaim, A. A. (1987) Monoclonal antibodies and synthetic tumor-associated glycoconjugates in the study of the expression of Thomsen-Friedenreich-like and Tn-like antigens on human cancers. J Natl Cancer Inst 78, 489-496
52. Thurnher, M., Clausen, H., Sharon, N., and Berger, E. G. (1993) Use of O-glycosylation-defective human lymphoid cell lines and flow cytometry to delineate the specificity of Moluccella laevis lectin and monoclonal antibody 5F4 for the Tn antigen (GalNAc alpha 1-O-Ser/Thr). Immunol Lett 36, 239-243
53. Reis, C. A., Sorensen, T., Mandel, U., David, L., Mirgorodskaya, E., Roepstorff, P., Kihlberg, J., Hansen, J. E., and Clausen, H. (1998) Development and characterization of an antibody directed to an alpha-N-acetyl-D-galactosamine glycosylated MUC2 peptide. Glycoconj J 15, 51-62
54. Danussi, C., Coslovi, A., Campa, C., Mucignat, M. T., Spessotto, P., Uggeri, F., Paoletti, S., and Colombatti, A. (2009) A newly generated functional antibody identifies Tn antigen as a novel determinant in the cancer cell-lymphatic endothelium interaction. Glycobiology 19, 1056-1067
55. Mazal, D., Lo-Man, R., Bay, S., Pritsch, O., Deriaud, E., Ganneau, C., Medeiros, A., Ubillos, L., Obal, G., Berois, N., Bollati-Fogolin, M., Leclerc, C., and Osinaga, E. (2013) Monoclonal antibodies toward different Tn-amino acid backbones display distinct recognition patterns on human cancer cells. Implications for effective immuno-targeting of cancer. Cancer Immunol Immunother 62, 1107-1122
56. Persson, N., Stuhr-Hansen, N., Risinger, C., Mereiter, S., Polonia, A., Polom, K., Kovacs, A., Roviello, F., Reis, C. A., Welinder, C., Danielsson, L., Jansson, B., and Blixt, O. (2017) Epitope mapping of a new anti-Tn antibody detecting gastric cancer cells. Glycobiology 27, 635-645
57. Naito, S., Takahashi, T., Onoda, J., Uemura, S., Ohyabu, N., Takemoto, H., Yamane, S., Fujii, I., Nishimura, S. I., and Numata, Y. (2017) Generation of Novel Anti-MUC1 Monoclonal Antibodies with Designed Carbohydrate Specificities Using MUC1 Glycopeptide Library. ACS Omega 2, 7493-7505
58. Sakai, K., Yuasa, N., Tsukamoto, K., Takasaki-Matsumoto, A., Yajima, Y., Sato, R., Kawakami, H., Mizuno, M., Takayanagi, A., Shimizu, N., Nakata, M., and Fujita-Yamaguchi, Y. (2010) Isolation and characterization of antibodies against three consecutive Tn-antigen clusters from a phage library displaying human single-chain variable fragments. J Biochem 147, 809-817
59. Kubota, T., Matsushita, T., Niwa, R., Kumagai, I., and Nakamura, K. (2010) Novel anti-Tn single-chain Fv-Fc fusion proteins derived from immunized phage library and antibody Fc domain. Anticancer Res 30, 3397-3405
60. Morita, N., Yajima, Y., Asanuma, H., Nakada, H., and Fujita-Yamaguchi, Y. (2009) Inhibition of cancer cell growth by anti-Tn monoclonal antibody MLS128. Biosci Trends 3, 32-37
61. Hubert, P., Heitzmann, A., Viel, S., Nicolas, A., Sastre-Garau, X., Oppezzo, P., Pritsch, O., Osinaga, E., and Amigorena, S. (2011) Antibody-dependent cell cytotoxicity synapses form in mice during tumor-specific antibody immunotherapy. Cancer Res 71, 5134-5143
62. Avichezer, D., Springer, G. F., Schechter, B., and Arnon, R. (1997) Immunoreactivities of polyclonal and monoclonal anti-T and anti-Tn antibodies with human carcinoma cells, grown in vitro and in a xenograft model. Int J Cancer 72, 119-127
63. Sedlik, C., Heitzmann, A., Viel, S., Ait Sarkouh, R., Batisse, C., Schmidt, F., De La Rochere, P., Amzallag, N., Osinaga, E., Oppezzo, P., Pritsch, O., Sastre-Garau, X., Hubert, P., Amigorena, S., and Piaggio, E. (2016) Effective antitumor therapy based on a novel antibody-drug conjugate targeting the Tn carbohydrate antigen. Oncoimmunology 5, el171434
64. Nakamoto, Y., Saga, T., Sakahara, H., Yao, Z., Zhang, M., Sato, N., Zhao, S., Nakada, H., Yamashina, I., and Konishi, J. (1998) Three-step tumor imaging with biotinylated monoclonal antibody, streptavidin and 111In-DTPA-biotin. Nucl Med Biol 25, 95-99
65. Campos, D., Freitas, D., Gomes, J., Magalhaes, A., Steentoft, C., Gomes, C., Vester-Christensen, M. B., Ferreira, J. A., Afonso, L. P., Santos, L. L., Pinto de Sousa, J., Mandel, U., Clausen, H., Vakhrushev, S. Y., and Reis, C. A. (2015) Probing the O-glycoproteome of gastric cancer cell lines for biomarker discovery. Mol Cell Proteomics 14, 1616-1629
66. Joshi, H. J., Jorgensen, A., Schjoldager, K. T., Halim, A., Dworkin, L. A., Steentoft, C., Wandall, H. H., Clausen, H., and Vakhrushev, S. Y. (2018) GlycoDomainViewer: a bioinformatics tool for contextual exploration of glycoproteomes. Glycobiology 28, 131-136
67. Steentoft, C., Vakhrushev, S. Y., Vester-Christensen, M. B., Schjoldager, K. T., Kong, Y., Bennett, E. P., Mandel, U., Wandall, H., Levery, S. B., and Clausen, H. (2011) Mining the O-glycoproteome using zinc-finger nuclease-glycoengineered SimpleCell lines. Nat Methods 8, 977-982
68. Tang, Y. A., Chen, Y. F., Bao, Y., Mahara, S., Yatim, S., Oguz, G., Lee, P. L., Feng, M., Cai, Y., Tan, E. Y., Fong, S. S., Yang, Z. H., Lan, P., Wu, X. J., and Yu, Q. (2018) Hypoxic tumor microenvironment activates GLI2 via HIF-lalpha and TGF-beta2 to promote chemoresistance in colorectal cancer. Proc Natl Acad Sci USA 115, E5990-E5999
69. White, M. A., Tsouko, E., Lin, C., Rajapakshe, K., Spencer, J. M., Wilkenfeld, S. R., Vakili, S. S., Pulliam, T. L., Awad, D., Nikolos, F., Katreddy, R. R., Kaipparettu, B. A., Sreekumar, A., Zhang, X., Cheung, E., Coarfa, C., and Frigo, D. E. (2018) GLUT12 promotes prostate cancer cell growth and is regulated by androgens and CaMKK2 signaling. Endocr Relat Cancer 25, 453-469
70. Chai, A. W., Cheung, A. K., Dai, W., Ko, J. M., Ip, J. C., Chan, K. W., Kwong, D. L., Ng, W. T., Lee, A. W., Ngan, R. K., Yau, C. C., Tung, S. Y., Lee, V. H., Lam, A. K., Pillai, S., Law, S., and Lung, M. L. (2016) Metastasis-suppressing NID2, an epigenetically-silenced gene, in the pathogenesis of nasopharyngeal carcinoma and esophageal squamous cell carcinoma. Oncotarget 7, 78859-78871
71. Wang, Q., Shen, B., Chen, L., Zheng, P., Feng, H., Hao, Q., Liu, X., Liu, L., Xu, S., Chen, J., and Teng, J. (2015) Extracellular calumenin suppresses ERK1/2 signaling and cell migration by protecting fibulin-1 from MMP-13-mediated proteolysis. Oncogene 34, 1006-1018
72. Rostami, N., Nikkhoo, A., Ajjoolabady, A., Azizi, G., Hojjat-Farsangi, M., Ghalamfarsa, G., Yousefi, B., Yousefi, M., and Jadidi-Niaragh, F. (2019) S1PR1 as a Novel Promising Therapeutic Target in Cancer Therapy. Mol Diagn Ther
73. Sheng, Y. H., He, Y., Hasnain, S. Z., Wang, R., Tong, H., Clarke, D. T., Lourie, R., Oancea, I., Wong, K. Y., Lumley, J. W., Florin, T. H., Sutton, P., Hooper, J. D., McMillan, N. A., and McGuckin, M. A. (2017) MUC13 protects colorectal cancer cells from death by activating the NF-kappaB pathway and is a potential therapeutic target. Oncogene 36, 700-713
74. Wagner, K. W., Punnoose, E. A., Januario, T., Lawrence, D. A., Pitti, R. M., Lancaster, K., Lee, D., von Goetz, M., Yee, S. F., Totpal, K., Huw, L., Katta, V., Cavet, G., Hymowitz, S. G., Amler, L., and Ashkenazi, A. (2007) Death-receptor O-glycosylation controls tumor-cell sensitivity to the proapoptotic ligand Apo2L/TRAIL. Nat Med 13, 1070-1077
75. Kanoh, A., Takeuchi, H., Kato, K., Waki, M., Usami, K., and Irimura, T. (2008) Interleukin-4 induces specific pp-GalNAc-T expression and alterations in mucin O-glycosylation in colonic epithelial cells. Biochim Biophys Acta 1780, 577-584
76. Park, J. H., Nishidate, T., Kijima, K., Ohashi, T., Takegawa, K., Fujikane, T., Hirata, K., Nakamura, Y., and Katagiri, T. (2010) Critical roles of mucin 1 glycosylation by transactivated polypeptide N-acetylgalactosaminyltransferase 6 in mammary carcinogenesis. Cancer Res 70, 2759-276932
77. Taniuchi, K., Cerny, R. L., Tanouchi, A., Kohno, K., Kotani, N., Honke, K., Saibara, T., and Hollingsworth, M. A. (2011) Overexpression of GalNAc-transferase GalNAc-T3 promotes pancreatic cancer cell growth. Oncogene 30, 4843-4854
78. Park, J. H., Katagiri, T., Chung, S., Kijima, K., and Nakamura, Y. (2011) Polypeptide N-acetylgalactosaminyltransferase 6 disrupts mammary acinar morphogenesis through O-glycosylation of fibronectin. Neoplasia 13, 320-326
79. Wu, Y. M., Liu, C. H., Hu, R. H., Huang, M. J., Lee, J. J., Chen, C. H., Huang, J., Lai, H. S., Lee, P. H., Hsu, W. M., Huang, H. C., and Huang, M. C. (2011) Mucin glycosylating enzyme GALNT2 regulates the malignant character of hepatocellular carcinoma by modifying the EGF receptor. Cancer Res 71, 7270-7279
80. Saeland, E., Belo, A. I., Mongera, S., van Die, I., Meijer, G. A., and van Kooyk, Y. (2012) Differential glycosylation of MUC1 and CEACAM5 between normal mucosa and tumour tissue of colon cancer patients. Int J Cancer 131, 117-128
81. Matsumoto, Y., Zhang, Q., Akita, K., Nakada, H., Hamamura, K., Tsuchida, A., Okajima, T., Furukawa, K., Urano, T., and Furukawa, K. (2013) Trimeric Tn antigen on syndecan 1 produced by ppGalNAc-T13 enhances cancer metastasis via a complex formation with integrin alpha5beta1 and matrix metalloproteinase 9. J Biol Chem 288, 24264-24276
82. Ho, W. L., Chou, C. H., Jeng, Y. M., Lu, M. Y., Yang, Y. L., Jou, S. T., Lin, D. T., Chang, H. H., Lin, K. H., Hsu, W. M., and Huang, M. C. (2014) GALNT2 suppresses malignant phenotypes through IGF-1 receptor and predicts favorable prognosis in neuroblastoma. Oncotarget 5, 12247-12259
83. Song, K., Herzog, B. H., Fu, J., Sheng, M., Bergstrom, K., McDaniel, J. M., Kondo, Y., McGee, S., Cai, X., Li, P., Chen, H., and Xia, L. (2015) Loss of Core 1-derived O-Glycans Decreases Breast Cancer Development in Mice. J Biol Chem 290, 20159-20166
84. Hofmann, B. T., Schluter, L., Lange, P., Mercanoglu, B., Ewald, F., Folster, A., Picksak, A. S., Harder, S., El Gammal, A. T., Grupp, K., Gungor, C., Drenckhan, A., Schluter, H., Wagener, C., Izbicki, J. R., Jucker, M., Bockhorn, M., and Wolters-Eisfeld, G. (2015) COSMC knockdown mediated aberrant O-glycosylation promotes oncogenic properties in pancreatic cancer. Mol Cancer 14, 109
85. Niang, B., Jin, L., Chen, X., Guo, X., Zhang, H., Wu, Q., Padhiar, A. A., Xiao, M., Fang, D., and Zhang, J. (2016) GalNAc-T4 putatively modulates the estrogen regulatory network through FOXA1 glycosylation in human breast cancer cells. Mol Cell Biochem 411, 393-402
86. Song, K. H., Park, M. S., Nandu, T. S., Gadad, S., Kim, S. C., and Kim, M. Y. (2016) GALNT14 promotes lung-specific breast cancer metastasis by modulating self-renewal and interaction with the lung microenvironment. Nat Commun 7, 13796
87. Lin, T. C., Chen, S. T., Huang, M. C., Huang, J., Hsu, C. L., Juan, H. F., Lin, H. H., and Chen, C. H. (2017) GALNT6 expression enhances aggressive phenotypes of ovarian cancer cells by regulating EGFR activity. Oncotarget 8, 42588-42601
88. Posey, A. D., Jr., Schwab, R. D., Boesteanu, A. C., Steentoft, C., Mandel, U., Engels, B., Stone, J. D., Madsen, T. D., Schreiber, K., Haines, K. M., Cogdill, A. P., Chen, T. J., Song, D., Scholler, J., Kranz, D. M., Feldman, M. D., Young, R., Keith, B., Schreiber, H., Clausen, H., Johnson, L. A., and June, C. H. (2016) Engineered CAR T Cells Targeting the Cancer-Associated Tn-Glycoform of the Membrane Mucin MUC1 Control Adenocarcinoma. Immunity 44, 1444-1454

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. An isolated antibody, comprising:
(i) a heavy chain variable domain (VH), which comprises a heavy chain complementary determining region 1 (HC CDR1) of SEQ ID NO: 1; a heavy chain complementary determining region 2 (HC CDR2) of SEQ ID NO: 2; and a heavy chain complementary determining region 3 (HC CDR3) of SEQ ID NO: 3; and/or
(ii) a light chain variable domain (VL), which comprises a light chain complementary determining region 1 (LC CDR1) of SEQ ID NO: 4; a light chain complementary determining region 2 (LC CDR2) of SEQ ID NO: 5; and a light chain complementary determining region 3 (LC CDR3) of SEQ ID NO: 6;
wherein the isolated antibody binds to Tn antigen.

2. An isolated antibody that binds to Tn antigen, wherein the antibody binds the same epitope as BaGs6 or competes against BaGs6 for binding to the Tn antigen.

3. The isolated antibody of claim 2, wherein the antibody comprises a HC CDR1, a HC CDR2, and a HC CDR3, each contains no more than 5, 4, 3, 2 or 1 amino acid variations as compared with the HC CDR1, HC CDR2, and HC CDR3 of BaGs6; and/or a LC CDR1, a LC CDR2, and a LC CDR3, each contains no more than 5, 4, 3, 2 or 1 amino acid variations as compared with the LC CDR1, LC CDR2, and LC CDR3 of BaGs6.

4. The isolated antibody of claim 2 or 3, wherein the antibody comprises a HC CDR1, a HC CDR2, and a HC CDR3, which collectively contains no more than 10, 9, 8, 7, 5, 4, 3, 2 or 1 amino acid variations as compared with the HC CDR1, HC CDR2, and HC CDR3 of BaGs6; and/or a LC CDR1, a LC CDR2, and a LC CDR3, which collectively contains no more than 10, 9, 8, 7, 5, 4, 3, 2 or 1 amino acid variations as compared with the LC CDR1, LC CDR2, and LC CDR3 of BaGs6.

5. The isolated antibody of any one of claims 1-4, wherein the antibody comprises the same heavy chain complementary determining regions (HC CDRs) and/or the same light chain complementary determining regions (LC CDRs) as BaGs6.

6. The isolated antibody of any one of claims 1-5, wherein the antibody comprises a heavy chain variable domain at least 85% identical to the heavy chain variable domain (VH) of BaGs6, and/or a light chain variable domain at least 85% identical to the light chain variable domain (VL) of BaGs6.

7. The isolated antibody of any one of claims 1-6, wherein the antibody comprises a heavy chain variable domain at least 85% identical to amino acid sequence of SEQ ID NO: 7, and a light chain variable domain at least 85% identical to amino acid sequence of SEQ ID NO: 8.

8. The isolated antibody of any one of claims 1-7, wherein the antibody is selected from the group consisting of a full-length antibody, a Fab fragment, a F(ab′) fragment, a F(ab′)2 fragment, a scFv, a Fv, a bi-specific antibody, or a single domain antibody.

9. The isolated antibody of claim 8, wherein the antibody is a full-length antibody.

10. The isolated antibody of claim 9, wherein the antibody is a mouse IgM.

11. The isolated antibody of claims 1-10, wherein the antibody comprises a heavy chain at least 85% identical to amino acid sequence of SEQ ID NO: 9, and a light chain at least 85% identical to amino acid sequence of SEQ ID NO: 10.

12. The isolated antibody of claim 9, wherein the antibody is a chimeric antibody.

13. The isolated antibody of claim 12, wherein the antibody is a chimeric antibody that comprises a human heavy chain constant region.

14. The isolated antibody of claim 12 or 13, wherein the antibody comprises a heavy chain constant region of the isotype IgG1, IgG2, IgG3, or IgG4.

15. The isolated antibody of claim 14, wherein the antibody comprises a heavy chain constant region of IgG1.

16. The isolated antibody of any one of claims 12-15, wherein the antibody comprises a human light chain constant region.

17. The isolated antibody of claim 12 or 13, wherein the antibody comprises a light chain constant region of the isotype κ and λ.

18. The isolated antibody of claim 17, wherein the antibody comprises a light chain constant region of the isotype κ.

19. The isolated antibody of any one of claims 12-18, wherein the antibody comprises a heavy chain at least 85% identical to amino acid sequence of SEQ ID NO: 11, and a light chain at least 85% identical to amino acid sequence of SEQ ID NO: 12.

20. The isolated antibody of any one of claims 1-19, wherein the antibody specifically binds Tn antigen on tumor cells.

21. The isolated antibody of any one of claims 1-20, wherein the antibody does not recognize Tn antigen on immunoglobulin A1 (IgA1).

22. The isolated antibody of claim 21, wherein the IgA1 is human IgA1.

23. The isolated antibody of any one of claims 1-22, wherein the antibody is afucosylated.

24. The isolated antibody of any one of claims 1-23, wherein the antibody is present in an antibody population, and wherein the antibody population comprises less than 50% of fucosylated antibodys.

25. The isolated antibody of claim 23 or 24, wherein the afucosylated antibody has increased Antibody-dependent cellular cytotoxicity (ADCC) activity compared to the same antibody that is fucosylated.

26. The isolated antibody of any one of claims 1-25, wherein the antibody is conjugated to an active agent.

27. The isolated antibody of claim 26, wherein the active agent is a particle, a nanoparticle, a surface, a small molecule, a peptide, an enzyme, an oligonucleotide, a detectable label, an imaging agent, or a therapeutic agent.

28. The isolated antibody of claim 27, wherein the imaging agent is a radioactive agent selected from the group consisting of fluorine-18, zirconium-89, copper-64, yttrium-86, indium-111, and iodine-124.

29. The isolated antibody of claim 27, wherein the therapeutic agent is a cytotoxic agent or a toxin.

30. The isolated antibody of claim 29, wherein the cytotoxic agent is selected from the group consisting of dolastin 10, zogamicin, monomethyl auristatin E (MMAE), cryptophycin and analogs thereof, enediyne antiobiotics, calicheamicin, capecitabine, lapatinib, anthracyclines, duocarmycins, and pyyrolobenzodiazepines.

31. The isolated antibody of claim 29, wherein the toxin is Pseudomonas exotoxin, or diphtheria toxin.

32. The isolated antibody of claim 27, wherein the detectable label is a fluorescent protein or a fluorescent compound.

33. A nucleic acid or a nucleic acid set, which collectively encode the isolated antibody of any one of claims 1-23.

34. The nucleic acid or nucleic acid set of claim 33, wherein the nucleic acid or nucleic acid set is a vector or a vector set, and wherein the vector or vector set is an expression vector.

35. The nucleic acid or nucleic acid set of claim 33 or 34 comprising a nucleotide sequence at least 85% identical to any one of SEQ ID NOs: 13-16.

36. A host cell, comprising the nucleic acid or nucleic acid set of any one of claims 33-35.

37. The host cell of claim 36, wherein the host cell is selected from the group consisting of a bacterial cell, a yeast cell, an insect cell, a plant cell, or a mammalian cell.

38. A genetically engineered host cell, comprising a double allele knock-out of a fucose synthase.

39. The genetically engineered host cell of claim 33, wherein the fucose synthase is a GDP-L-fucose synthase.

40. The genetically engineered host cell of claim 38 or 39, wherein the cells are capable of producing afucosylated antibodies in the absence of fucose in cell culture medium.

41. The genetically engineered host cell of claim 38 or 39, wherein the cells are capable of producing fucosylated antibodies in the presence of fucose in cell culture medium.

42. The genetically engineered host cell of any one of claims 38-41, wherein the antibody is a therapeutic antibody.

43. The genetically engineered host cell of any one of claims 38-41, wherein the antibody is a non-therapeutic antibody.

44. The genetically engineered host cell of any one of claims 38-43, comprising the nucleic acid or nucleic acid set of claims 28-29.

45. The genetically engineered host cell of any one of claims 32-44, wherein the antibody is the anti-Tn antigen antibody of claims 1-32.

46. The genetically engineered host cell of any one of claims 38-45, wherein the genetically engineered host cell is a mammalian cell.

47. The genetically engineered host cell of claim 46, wherein the mammalian cell is HEK293 cell, Chinese hamster ovary (CHO) cell, dhFr− CHO cell, HeLa cell, HT-1080 cell, PER.C6, HKB-11 cell, CAP cell, HuH07 cell, NS0 cell, HKB11, Sp2/0 cell, BHK cell, or C127 cells.

48. A genetically engineered immune cell, which expresses a chimeric receptor comprising an extracellular domain and at least one cytoplasmic signaling domain, wherein the extracellular domain is a single chain antibody derived from the antibody of any one of claims 1-32.

49. The genetically engineered immune cell of claim 48, wherein the single chain antibody comprises a heavy chain variable domain and/or a light chain variable domain as set forth in any one of claims 1-7.

50. The genetically engineered immune cell of claim 48 or 49, wherein the genetically engineered immune cell is a CAR-T cell or a CAR-NK cell.

51. A pharmaceutical composition, comprising the isolated antibody of any one of claims 1-32, the nucleic acid of any one of claims 32-35, the host cell of claim 36 or 37, the genetically engineered host cell of any one of claims 38-47, or the genetically engineered immune cell of any one of claims 48-50, wherein the pharmaceutical composition optionally further comprises a pharmaceutically acceptable carrier.

52. A kit comprising the isolated antibody of any one of claims 1-32, the nucleic acid of any one of claims 32-35, the host cell of claim 36 or 37, the genetically engineered host cell of any one of claims 38-47, or the genetically engineered immune cell of any one of claims 48-50, or the pharmaceutical composition of claim 51.

53. A method for producing an antibody that binds to human Tn antigen, the method comprising:
(i) culturing the host cell of claim 36 or 37, or the genetically engineered host cell of claims 38-37 in a medium for production of the antibody; and
(ii) collecting the host cell or the medium for isolation of the antibody.

54. The method of claim 53, further comprising purifying the antibody from the host cell or the medium.

55. A method for detecting the presence of Tn antigen, the method comprising contacting an anti-Tn antigen antibody of any one of claims 1-32 with a subject or a biological sample obtained from a subject suspected of containing a Tn antigen, and determining binding of the anti-Tn antigen antibody to Tn antigen in the sample.

56. The method of claim 55, wherein the antibody is conjugated to a detectable label.

57. The method of claim 55 or 56, wherein the biological sample is in vivo and the contacting step is performed by administering the subject an effective of the anti-Tn antigen antibody.

58. A method for treating a cancer in a patient in need thereof, the method comprising administering to a subject an effective amount of the isolated antibody of any one of claims 1-32, the nucleic acid of any one of claims 32-35, the host cell of claim 36 or 37, the genetically engineered host cell of any one of claims 38-47, or the genetically engineered immune cell of any one of claims 48-50, the pharmaceutical composition of claim 51, or the kit of claim 52.

59. The method of claim 58, wherein the subject is a human patient having, suspected of having, or at risk for cancer.

60. The method of claim 58 or 59, wherein the human patient has a cancer selected from the group consisting of colorectum cancer, breast cancer, prostate cancer, lung cancer, ovarian cancer, stomach cancer, bladder cancer, cervix cancer, pancreatic cancer, endometrial cancer, glioblastomas, salivary gland cancer, nasopharyngeal cancers, skin cancers, basal cell carcinomas, squamous cell carcinomas, renal cell carcinomas, ductal carcinomas, invasive ductal carcinomas, adenocarcinomas, esophageal cancer, unspecified gastrointestinal cancer, pancreatic cancer, melanoma, sarcomas, including angiosarcoma, bone sarcoma, osteosarcoma, neurofibrosarcomas, rhabdomyosarcoma, soft tissue sarcoma, synovial sarcoma, condrosarcoma, chordomas, Kaposi's sarcoma, giant cell tumor of the bone, leiomyosarcoma, desmoid-type fibromatosis, Ewing's sarcoma, fibroblastic sarcoma, gastrointestinal stromal tumors, lymphomas, leukemia, and thymomas.

61. A method for producing an afucosylated antibody comprising
(i) transfecting the genetically engineered host cells of claims 38-47 with a nucleic acid or a nucleic acid set encoding an antibody;
(ii) culturing the genetically engineered host cell in a medium for production of the antibody; and
(iii) collecting the genetically engineered host cell or the medium for isolation of the antibody.

62. The method of claim 61, wherein the antibody is a therapeutic antibody.

63. The method of claim 61 or 62, wherein the antibody is the anti-Tn antibody of claims 1-32.
